(19) United States
(12) Patent Application Publication
(10) Pub. No.: US 2023/0159973 A1
(43) Pub. Date: May 25, 2023

(54) BACTERIAL SYSTEM FOR PRODUCING HUMAN O-GLYCOPROTEINS

(71) Applicant: CORNELL UNIVERSITY, Ithaca, NY (US)

(72) Inventors: Matthew P. DELISA, Ithaca, NY (US); Aravind NATARAJAN, Menlo Park, CA (US)

(21) Appl. No.: 17/920,532

(22) PCT Filed: Apr. 23, 2021
(86) PCT No.: PCT/US2021/028901

§ 371 Date: Oct. 21, 2022

Related U.S. Application Data
(60) Provisional application No. 63/014,486, filed on Apr. 23, 2020.

(51) Int. Cl. C12P 21/00; C12N 9/90; C12N 9/12; C12N 9/10

(52) U.S. Cl. CPC C12P 21/005; C12N 9/90; C12Y 501/03002; C12N 9/1205; C12Y 207/0106; C12N 9/1048; C12Y 204/01122

(57) ABSTRACT

The present application relates to recombinant prokaryotic host cells expressing one or more 4-epimerases, one or more glycosyl-1-phosphate transferases, one or more O-oligosaccharyltransferases, and, optionally, one or more ß1,3-galactosyltransferase enzymes capable of transferring galactose to undecaprenyl pyrophosphate-linked N-Acetylgalactosamine. Also disclosed are methods for producing an O-glycosylated protein.

This application claims the priority benefit of U.S. Provisional Patent Application Ser. No. 63/014,486, filed Apr. 23, 2020, which is hereby incorporated by reference in its entirety.

This invention was made with government support under 1163167 awarded by Defense Threat Reduction Agency, 1605242 awarded by National Science Foundation Division of Chemical, Bioengineering, Environmental and Transport Systems, 1R01GM127578-01 awarded by National Institutes of Health, 1U54CA210184-01 awarded by National Institutes of Health. The government has certain rights in the invention.

### FIELD

The present disclosure relates to recombinant prokaryotic host cells and methods for producing an O-glycosylated protein.

### BACKGROUND

Protein glycosylation is one of the most abundant and structurally complex posttranslational modifications (PTMs) (Khoury et al., “Proteome-Wide Post-Translational Modification Statistics: Frequency Analysis and Curation of the Swiss-Prot Database,” Sci. Rep. 1:90 (2011) and Walsh et al., “Protein Posttranslational Modifications: The Chemistry of Proteome Diversifications,” Angew Chem. Int. Ed. Engl. 44:7342-7372 (2005)) and occurs in all domains of life (Abu-Qarn et al., “Not Just for Eukarya Anymore: Protein Glycosylation in Bacteria and Archaea,” Curr. Opin. Struct Biol. 18:544-550 (2008)). Protein-linked glycans (mono-, oligo- or polysaccharide) play important roles in protein folding, solubility, stability, serum half-life, immunogenicity, and biological function (Varki, A., “Biological Roles of Glycans,” Glycobiology 27:3-49 (2017)). Glycan conjugation is also critical to the development of many biologics, with glycoproteins accounting for more than 70% of current protein-based drugs (Sethuraman & Stadheim, “Challenges in Therapeutic Glycoprotein Production,” Curr. Opin. Biotechnol. 17:341-346 (2006)) and glycoconjugate vaccines representing one of the safest and most successful vaccination approaches developed over the last 40 years (Rappuoli, R. “Glycoconjugate Vaccines: Principles and Mechanisms,” Sci. Trans. Med. 10:eaat4615 (2018)). The importance of glycosylation in both nature and the clinic has prompted widespread glycoengineering efforts that seek to: (i) create designer production platforms for controllable glycoprotein synthesis (Valderrama-Rincon et al., An Engineered Eukaryotic Protein Glycosylation Pathway in Escherichia coli,” Nat. Chem. Biol. 8:434-436 (2012); Meuris et al., “GlycoDelete Engineering of Mammalian Cells Simplifies N-Glycosylation of Recombinant Proteins,” Nat. Biotechnol. 32:485-489 (2014); Hamilton et al., “Production of Complex Human Glycoproteins in Yeast,” Science 301:1244-1246 (2003); Jaroentomeechai et al., “Single-Pot Glycoprotein Biosynthesis Using a Cell-Free Transcription-Translation System Enriched with Glycosylation Machinery,” Nat. Commun. 9:2686 (2018); Kightlinger et al., “A Cell-Free Biosynthesis Platform for Modular Construction of Protein Glycosylation Pathways,” Nat. Commun. 10:5404 (2019); Feldman et al., “Engineering N-Linked Protein Glycosylation with Diverse O Antigen Lipopolysaccharide Structures in Escherichia coli,” Proc. Natl. Acad Sci. USA 102:3016-3021 (2005); Tytgat et al., “Cytoplasmic Glycoengineering Enables Biosynthesis of Nanoscale Glycoprotein Assemblies,” Nat. Commun. 10:5403 (2019); Aumiller et al., “A Transgenic Insect Cell Line Engineered to Produce CMP-Sialic Acid and Sialylated Glycoproteins,” Glycobiology 13:497-507 (2003); Chang et al., “Small-Molecule Control of Antibody N-Glycosylation in Engineered Mammalian Cells,” Nat. Chem. Biol. 15:730-736 (2019); and Yang et al., “Engineering Mammalian Mucin-Type O-Glycosylation in Plants,” J. Biol. Chem. 287:11911-11923 (2012)); and (ii) rationally manipulate glycan structures and their attachment sites as a means to optimize the therapeutic and immunologic properties of proteins (Elliott et al., “Enhancement of Therapeutic Protein In Vivo Activities Through Glycoengineering,” Nat. Biotechnol. 21:414-421 (2003); Huang et al., “Chemoenzymatic Glycoengineering of Intact IgG Antibodies for Gain of Functions,”J. Am. Chem. Soc. 134:12308-12318 (2012); Broecker et al., “Multivalent Display of Minimal Clostridium difficile Glycan Epitopes Mimics Antigenic Properties of Larger Glycans,” Nat. Commun. 7:11224 (2016); Umana et al., “Engineered Glycoforms of an Antineuroblastoma IgG1 with Optimized Antibody-Dependent Cellular Cytotoxic Activity,” Nat. Biotechnol. 17:176-180 (1999); and Ilyushin et al., “Chemical Polysialylation of Human Recombinant Butyrylcholinesterase Delivers a Long-Acting Bioscavenger for Nerve Agents In Vivo,” Proc. Natl. Acad. Sci. USA 110:1243-1248 (2013)).

Genetically engineered eukaryotic expression hosts have provided extensive access to a chemically rich landscape of glycoproteins enabling efforts to generate defined glycoprotein epitopes and engineer proteins with advantageous properties (Meuris et al., “GlycoDelete Engineering of Mammalian Cells Simplifies N-Glycosylation of Recombinant Proteins,” Nat. Biotechnol. 32:485-489 (2014); Hamilton et al., “Production of Complex Human Glycoproteins in Yeast,” Science 301:1244-1246 (2003); Aumiller et al., “A Transgenic Insect Cell Line Engineered to Produce CMP-Sialic Acid and Sialylated Glycoproteins,” Glycobiology 13:497-507 (2003); Chang et al., “Small-Molecule Control of Antibody N-Glycosylation in Engineered Mammalian Cells,” Nat. Chem. Biol. 15:730-736 (2019); and Yang et al., “Engineering Mammalian Mucin-Type O-Glycosylation in Plants,” J. Biol. Chem. 287:11911-11923 (2012)). However, glycoengineering in eukaryotes is complicated by the fact that glycans are synthesized across several subcellular compartments by the coordinated activities of numerous glycosyltransferases (GTs) (Schwarz & Aebi, “Mechanisms and Principles of N-Linked Protein Glycosylation,” Curr. Opin. Struct. Biol. 21:576-582 (2011)) and that glycosylation is an essential process, with significant alteration of glycosylation pathways often leading to severe fitness defects (Choi et al., “Use of Combinatorial Genetic Libraries to Humanize N-Linked Glycosylation in the Yeast Pichia pastoris,” Proc. Natl. Acad. Sci. USA 100:5022-5027 (2003)). Glycoengineering in bacteria, on the other hand, is not constrained by these issues due to the non-essential nature of protein glycosylation in bacterial cells and thus has emerged as an attractive alternative that permits customizable glycan construction and protein glycosylation (Natarajan et al., “Metabolic Engineering of Glycoprotein Biosynthesis in Bacteria,” Emerg. Top Life Sci. 2: 419-432 (2018)). Moreover, some bacteria including laboratory strains of Escherichia coli lack endogenous glycosylation pathways, thereby providing a “clean” chassis for installation of orthogonal glycosylation pathways with little to no interference from endogenous GTs and the potential for more uniformly glycosylated protein products.

Over the last two decades, numerous efforts have collectively endowed E. coli and E. coli-derived cell-free extracts with the catalytic potential to produce diverse N-glycoproteins. Notably, this includes generation of structurally complex glycans, such as the eukaryotic Man₃GlcNAc₂ structure (Valderrama-Rincon et al., An Engineered Eukaryotic Protein Glycosylation Pathway in Escherichia coli,” Nat. Chem. Biol. 8:434-436 (2012)), and their installation at authentic human glycosites (Ollis et al., “Engineered Oligosaccharyltransferases with Greatly Relaxed Acceptor-Site Specificity,” Nat. Chem. Biol. 10:816-822 (2014)). In contrast, the analogous construction of O-linked glycosylation pathways in bacteria has received relatively little attention. Two of the earliest examples involved reconstituting the initiating step of vertebrate mucin-type O-glycosylation in E. coli (Henderson et al., “Site-Specific Modification of Recombinant Proteins: A Novel Platform for Modifying Glycoproteins Expressed in E. coli,” Bioconjug. Chem. 22:903-912 (2011) and Mueller et al., “High level In Vivo Mucin-Type Glycosylation in Escherichia coli,” Microb. Cell Fact. 17:168 (2018). Specifically, human polypeptide N-acetylgalactosaminyl-transferase 2 (GalNAcT2) was used to conjugate GalNAc onto threonine residues of peptides derived from different O-glycoproteins including human mucin 1 (MUC1) or an artificial rat-derived MUC10 in the cytoplasm of E. coli. Most recently, it was shown that the GalNAc installed by GalNAcT2 on threonine residues could be extended by a single galactose (Gal) residue using Campylobacter jejuni β1,3-galactosyltransferase CgtB, yielding acceptor proteins modified with Gal-β1,3-GalNAcα (T antigen or core 1) (Du et al., “A Bacterial Expression Platform for Production of Therapeutic Proteins Containing Human-Like O-Linked Glycans,” Cell Chem. Biol. 26:203-212 e205 (2019)). Bacterial protein O-glycosylation pathways have also been successfully reconstituted in E. coli; however, these systems are unlike the processive mechanism used by eukaryotes and instead operate according to an en bloc mechanism that is reminiscent of the canonical N-glycosylation process (Natarajan et al., “Metabolic Engineering of Glycoprotein Biosynthesis in Bacteria,” Emerg. Top Life Sci. 2: 419-432 (2018)).

The present application is directed to overcoming these and other deficiencies in the art.

### SUMMARY

Accordingly, a first aspect of the present disclosure relates to a recombinant prokaryotic host cell expressing one or more 4-epimerases, one or more glycosyl-1-phosphate transferases, and one or more O-oligosaccharyltransferases.

Another aspect of the present disclosure relates to a recombinant prokaryotic host cell expressing one or more 4-epimerases, one or more glycosyl-1-phosphate transferases, one or more O-oligosaccharyltransferases, and one or more ß1,3-galactosyltransferase enzymes capable of transferring galactose (Gal) to undecaprenyl pyrophosphate (Und-PP)-linked N-Acetylgalactosamine (GalNAc).

Another aspect of the present disclosure relates to a method for producing an O-glycosylated protein. This method involves providing a recombinant host cell expressing one more 4-epimerases, one or more glycosyl-1-phosphate transferases, one or more O-oligosaccharyltransferases, and a glycoprotein target comprising one or more serine and/or threonine residues. This method further involves culturing the host cell under conditions effective to: (i) produce N-acetylgalactosamine (GalNAc) linked to undecaprenyl pyrophosphate (Und-PP); and (ii) transfer the N-acetylgalactosamine (GalNAc) linked to undecaprenyl pyrophosphate (Und-PP) en bloc to a serine or threonine amino acid of the glycoprotein target.

Another aspect of the present disclosure relates to a method for producing an O-glycosylated protein. This method involves providing a recombinant host cell expressing one more 4-epimerases, one or more glycosyl-1-phosphate transferases, one or more O-oligosaccharyltransferases, one or more ß1,3-galactosyltransferase enzymes capable of transferring galactose (Gal) to undecaprenyl pyrophosphate (Und-PP)-linked N-Acetylgalactosamine (GalNAc), and a glycoprotein target comprising one or more serine and/or threonine residues. This method further involves culturing said host cell under conditions effective to: (i) produce N-acetylgalactosamine (GalNAc) linked to undecaprenyl pyrophosphate (Und-PP); (ii) extend Und-PP-GalNAc by a single galactose (Gal) monosaccharide to yield lipid-linked Gal-ß1,3-GalNAc; and (iii) transfer the lipid-linked Gal-ß1,3-GalNAc en bloc to a serine or threonine amino acid of the glycoprotein target.

Another aspect of the present disclosure relates to an in vitro method for producing an O-glycosylated protein. This method involves providing glycosylation reagents comprising one more 4-epimerases, one or more glycosyl-1-phosphate transferases, and one or more O-oligosaccharyltransferases; providing a glycoprotein target comprising one or more serine and/or threonine residues; and incubating said glycosylation reagents and said glycoprotein target under conditions effective to: (i) yield N-acetylgalactosamine (GalNAc) linked to undecaprenyl pyrophosphate (Und-PP), and (ii) transfer the N-acetylgalactosamine (GalNAc) linked to undecaprenyl pyrophosphate (Und-PP) en bloc to a serine or threonine amino acid of the glycoprotein target.

Another aspect of the present disclosure relates to an in vitro method for producing an O-glycosylated protein. This method involves providing glycosylation reagents comprising one more 4-epimerase enzymes, one or more heterologous diacetylbacilliosaminyl-1-phosphate transferase enzymes, one or more heterologous O-oligosaccharyltransferases, and one or more ß1,3-galactosyltransferase enzymes capable of transferring galactose (Gal) to undecaprenyl pyrophosphate (Und-PP)-linked N-Acetylgalactosamine (GalNAc); providing a glycoprotein target comprising one or more serine and/or threonine residues; and incubating said glycosylation reagents and said glycoprotein target under conditions effective to: (i) yield lipid-linked Gal-ß1,3-GalNAc, and (ii) transfer the lipid-linked Gal-ß1,3-GalNAc and any additional sugars en bloc to a serine or threonine amino acid of the glycoprotein target.

Another aspect of the present disclosure relates to an in vitro method for producing an O-glycosylated protein. This method involves providing reagents suitable for synthesizing a glycoprotein target; providing glycosylation reagents comprising one more 4-epimerases, one or more glycosyl-1-phosphate transferases, and one or more O-oligosaccharyltransferases; providing a nucleic acid molecule encoding a glycoprotein target and incubating said reagents suitable for synthesizing a glycoprotein target, glycosylation reagents, and nucleic acid molecule encoding a glycoprotein target under conditions effective to: (i) synthesize the glycoprotein target encoded by the nucleic acid molecule encoding a glycoprotein target, (i) yield N-acetylgalactosamine (GalNAc) linked to undecaprenyl pyrophosphate (Und-PP), and (ii) transfer the N-acetylgalactosamine (GalNAc) linked to undecaprenyl pyrophosphate (Und-PP) en bloc to a serine or threonine amino acid of the glycoprotein target.

Another aspect of the present disclosure relates to an in vitro method for producing an O-glycosylated protein. This method involves providing reagents suitable for synthesizing a glycoprotein target; providing glycosylation reagents comprising one more 4-epimerase enzymes, one or more heterologous N,N′-diacetylbacilliosaminyl-1-phosphate transferase enzymes, one or more heterologous O-oligosaccharyltransferases, and one or more ß1,3-galactosyltransferase enzymes capable of transferring galactose (Gal) to undecaprenyl pyrophosphate (Und-PP)-linked N-Acetylgalactosamine (GalNAc), providing a nucleic acid molecule encoding a glycoprotein target; and incubating said reagents suitable for synthesizing a glycoprotein target, glycosylation reagents, and nucleic acid molecule encoding a glycoprotein target under conditions effective to: (i) synthesize the glycoprotein target encoded by the nucleic acid molecule encoding a glycoprotein target, (ii) yield lipid-linked Gal-ß1,3-GalNAc, and (iii) transfer the lipid-linked Gal-ß1,3-GalNAc and any additional sugars en bloc to a serine or threonine amino acid of the glycoprotein target.

Another aspect of the present disclosure relates to a prokaryotic host cell expressing an α2,6-sialyltransferases and an α2,3-sialyltransferase, where the α2,6-sialyltransferases is the α2,6-sialyltransferases from Photobacterium sp. JT-ISH-224 (PspST6) and where the α2,3-sialyltransferase is the α2,3-sialyltransferase from E. coli O104 (EcWbwA).

Another aspect of the present disclosure relates to a prokaryotic host cell expressing one or more 4-epimerases, one or more glycosyl-1-phosphate transferases, one or more O-Antigen ligases (e.g., EcWaaL), and, optionally, one or more ß1,3-galactosyltransferase enzymes capable of transferring galactose (Gal) to undecaprenyl pyrophosphate (Und-PP)-linked N-Acetylgalactosamine (GalNAc). In some embodiments according to this aspect of the invention, the prokaryotic host cell does not encode an O-oligosaccharyltransferase.

Another aspect of the present disclosure relates to a method for producing a lipid linked Gal-β1,3-GalNAcα (T antigen or core 1). This method involves providing a recombinant host cell expressing one or more 4-epimerases, one or more glycosyl-1-phosphate transferases, one or more ß1,3-galactosyltransferase enzymes capable of transferring galactose (Gal) to undecaprenyl pyrophosphate (Und-PP)-linked N-Acetylgalactosamine (GalNAc), and one or more O-Antigen ligases (e.g., EcWaaL). This method further involves culturing the host cell under conditions effective to: (1) produce Gal-β1,3-GalNAc linked to undecaprenyl pyrophosphate (Und-PP) and (ii) transfer Gal-β1,3-GalNAc linked to undecaprenyl pyrophosphate (Und-PP) en bloc to a lipid target.

The examples of the present disclosure demonstrate the implantation of a synthetic glycobiology approach to engineer E. coli with human-like O-glycosylation pathways based on the bacterial PglL/O paradigm. As proof-of-concept, a collection of orthogonal pathways for biosynthesis of proteins decorated with mucin-type O-glycans including Tn, T, sialyl-Tn (STn), and sialyl-T (ST) glycans were engineered. Each of these pathways involved cytoplasmic preassembly of desired O-glycan structures on Und-PP by a prescribed set of heterologous GTs expressed in E. coli cells metabolically engineered to produce required nucleotide sugar donors. The addition of heterologous O-OSTs enabled efficient site-directed O-glycosylation of acceptor sequences derived from different human glycoproteins.

Glycoengineered E. coli cells were also used to source crude cell extracts selectively enriched with O-glycosylation machinery, enabling a one-pot, cell-free reaction scheme for efficient and site-specific installation of O-glycans on target acceptor proteins. Overall, it is anticipated that the glycoengineered bacteria described herein will enable future efforts to produce structurally diverse O-glycoproteins for a variety of applications at the intersection of glycoscience, synthetic biology, and biomedicine.

### BRIEF DESCRIPTION OF THE DRAWINGS

FIGS. 1A-B are schematics showing natural and synthetic mucin-type O-glycosylation pathways. FIG. 1A is a schematic showing that vertebrate mucin-type O-glycan synthesis originates from the hydroxyl group of a serine or threonine (S/T) amino acid by the addition of N-acetylgalactosamine (GalNAc) by N-acetylgalactosaminyl-transferase 2 (GalNAcT2) to form the Tn antigen structure. ClGalT1 adds β1,3-linked galactose (Gal) to the initial GalNAcα-S/T to generate the T antigen. The Tn and T antigens can be further elaborated with GlcNAc and NeuNAc in a variety of ways, with a few illustrative examples shown in FIG. 1A. FIG. 1B is a representative schematic of an engineered pathway for orthogonal O-glycoprotein synthesis in E. coli. CjGne maintains a pool of UDP-GalNAc that serves as the activated nucleotide sugar donor for AbPglC, which catalyzes the formation of Und-PP-linked GalNAc. EcWbwC extends Und-PP-GalNAc by a single Gal residue, yielding lipid-linked Gal-β1,3-GalNAc. Following flipping of the LLO to the periplasmic face of the cytoplasmic membrane by the native E. coli flippase Wzx, the preassembled T antigen glycan is transferred en bloc to a serine amino acid on a Sec pathway-exported acceptor protein by an O-OST such as NgPglO or NmPglL. It is noted that the absence of EcWbwC enables generation of Tn-modified acceptor proteins while the further elaboration of Gal-β1,3-GalNAc with additional sugars such as NeuNAc followed by transfer to protein is also possible.

FIGS. 2A-2B demonstrate the biosynthesis of O-glycoproteins bearing Tn and T antigens. FIG. 2A shows immunoblot analysis of acceptor proteins purified from CLM25 (W3110 ΔwecA ΔwaaL) cells co-transformed with pOG-Tn (left panels) or pOG-T (right panels) without an O-OST (−), pOG-Tn-NgPglO, or pOG-Tn NmPglL along with pEXT-spDsbA-MBP^{MOOR} or pEXT-spDsbA-MBP^{MOORmut} as indicated. Absence of O-OST or mutation of acceptor serine to glycine in MBP^{MOORmut} served as controls. Blots were probed with anti-hexa-histidine antibody (6×His) to detect acceptor proteins and either WA or PNA lectin to detect the Tn or T antigen, respectively. Molecular weight (MW) markers are indicated on the left. Results are representative of at least three biological replicates. FIG. 2B are spectra showing the results of nano-LC-MS/MS analysis of purified acceptor protein generated by CLM25 cells carrying plasmid pOG-Tn-NgPglO (top spectrum) or pOG-T-NgPglO (bottom spectrum) and pEXT-spDsbA-MBP^{MOORmut} MOOR. Sequence coverage of 88% and 75% was obtained for glycosylated MBP^{MOOR} with Tn and T antigens, respectively, in the analysis. Spectrum for Tn glycoform reveals a dominant species (94% abundance) corresponding to peptide fragment bearing a single HexNAc and a less abundant (6%) aglycosylated species. Spectrum for T glycoform reveals a dominant species (86% abundance) corresponding to peptide fragment bearing a single HexHexNAc as well as two minor species bearing a single HexNAc and no modification (3% and 11% abundance, respectively). Sequence of detected peptide (SEQ ID NO: 37) is shown at top with arrow denoting modified serine (bold underline) as determined by EThcD fragmentation analysis.

FIGS. 3A-3C demonstrate orthogonal biosynthesis of sialylated O-glycans. FIG. 3A is a schematic of a glyco-recoding strategy for genomic integration of the CMP-NeuNAc biosynthetic pathway in E. coli. Genes encoding E. coli K1 neuDBAC were cloned in shuttle vector pRecO-PS, which was used to insert the neu operon in place of the O-PS pathway between glf and gnd in E. coli MC4100 strain background. FIG. 3B is a bar graph showing the results of LC-MS analysis of lysates derived from glyco-recoded cells, comparing intracellular CMP-NeuNAc levels measured in cells carrying plasmid-encoded copies of neuDBAC genes versus those carrying genomically integrated copy of neuDBAC. Cells lacking the neuDBAC genes served as controls. Data is the average of three biological replicates and error bars represent the standard deviation of the mean. FIG. 3C is a spectrum showing the results of nano-LC-MS/MS analysis of purified acceptor protein generated by glyco-recoded cells carrying plasmid pOG-T-NgPglO and pEXT-spDsbA-MBP^{MOOR}-EcWbwA. Sequence coverage of 94% was obtained for the MBP^{MOOR} protein in the analysis. Spectrum reveals a dominant species (70% abundance) corresponding to the indicated peptide fragment bearing a single NeuNAcHexHexNAc and two minor species bearing a single HexHexNAc and no modification (22% and 8% abundance, respectively). Sequence of detected peptide is shown at bottom with arrow denoting modified serine (bold underline) as determined by EThcD fragmentation analysis.

FIGS. 4A-4B are immunoblots demonstrating cell-free O-glycosylation using glyco-enriched extracts. FIG. 4A shows the results of immunoblot analysis of in vitro glycosylation (IVG) reactions that were performed by incubating purified MBP^{MOOR} or MBP^{MOORmut} acceptor proteins in the presence of crude membrane extracts (CMEs) prepared from CLM25 cells carrying pOG-T-NgPglO (+) or pOG-T without an O-OST (−). Glyco-enriched CMEs alone (lane 1) or glycosylated MBP^{MOOR} (gMBP^{MOOR}) that was previously purified from glycoengineered bacteria (lane 5) served as negative and positive controls, respectively. FIG. 4B shows the results of immunoblot analysis of acceptor proteins produced by integrated CFGpS in which transcription, translation, and O-glycosylation were performed altogether in a single reaction. Specifically, 1 ml reactions comprised of glyco-enriched S12 extract derived from CLM25 cells carrying pOG-T NgPglO were primed with plasmid pJL1-MBP^{MOOR} or pJL1-MBP^{MOORmut} as indicated. Blots in FIG. 4A and FIG. 4B were probed with anti-hexa-histidine antibody (6×His) to detect the acceptor proteins and PNA to detect the T antigen. Molecular weight (MW) markers are indicated on the left. Results are representative of at least three biological replicates.

FIGS. 5A-5B demonstrate the O-linked glycosylation of diverse protein targets. FIG. 5A shows the results of immunoblot analysis of acceptor proteins purified from CLM25 cells co-transformed with pOG-T-NgPglO (+) or pOG-T without NgPglO (−) along with pEXT-based plasmid encoding each of the different protein targets as indicated. Absence of NgPglO or mutation of acceptor serine to glycine in MBP^{MOORmut} served as negative controls. Blots were probed with anti-hexa-histidine antibody (6×His) to detect acceptor proteins and PNA lectin to detect the T antigen. Additional blot for MUC1 variants was probed with murine H23 antibody (anti-MUC1) that is specific for APDTRP motif in human MUC1. Shown at bottom are acceptor sequences derived from human EPO, GPC, and MUC1 as well as synthetic SAP: EPO (SEQ ID NO: 38); GPC (SEQ ID NO: 39); SAP (SEQ ID NO: 40); MUC1_8 (SEQ ID NO: 41); MUC1_12 (SEQ ID NO: 42); MUC1_16 (SEQ ID NO: 43); MUC1_20 (SEQ ID NO: 44); MUC_24 (SEQ ID NO: 45); and MUC1_41 (SEQ ID NO: 46). All acceptor motifs except for MUC1_41 are presented in the context of the hydrophilic flanking regions derived from the MOOR tag (underline). MUC_41 was designed without hydrophilic flanking residues and includes the VNTR region as indicated. Serine amino acids determined to be glycosylated by EThcD fragmentation analysis are shown in bold font. FIG. 5B shows the results of immunoblot analysis of MUC1_41 expressed in CLM25 cells carrying pOG-Tn-NgPglO (+) or pOG-Tn without NgPglO (−). Also shown is MBP^{MOOR} and MBP^{MOORmut} derived from the same cells. Blots were probed with anti-6×His antibody to detect acceptor proteins, VVA lectin to detect the Tn antigen, anti-MUC1 to detect MUC1_41, and chimeric 5E5 antibody (ch5E5) to detect Tn-MUC1. Arrow denotes the expected Tn-MUC1 glycoform, while asterisks denote higher and lower molecular weight species that may represent SDS-stable multimers and degradation products, respectively. Molecular weight (MW) markers are indicated on the left of each blot. All immunoblot results are representative of at least three biological replicates.

FIG. 6 demonstrates the FACS gating strategy. For all flow cytometric screening, cells were analyzed using a FACSCalibur flow cytometer (BD Biosciences), and at least 100,000 total events were recorded. The events from the unlabelled MCΔw control sample were analyzed using FlowJo 10.5, and gated based on forward scatter (FSC) and side scatter (SSC) to represent the E. coli cell population, minimizing artifacts from debris. This same gate was then applied to all samples, followed by calculation of the median fluorescent intensity.

FIG. 7 shows MS/MS fragmentation analysis of Tn-modified glycoprotein. EThcD fragmentation analysis of glycosylated peptide ³⁹⁷NVGGDLDWPAAAS(HexNAc)APQPGKPPR⁴¹⁸ (SEQ ID NO: 23) derived from MBP^{MOOR} by trypsin digestion. The spectrum Identifies the neutral loss pattern of the single HexNAc monosaccharide, corresponding oxonium ions, and fragments of the glycopeptide (c and z ions), validating the glycosylation and the site of glycosylation at S409 within the 8-residue WPAAASAP (SEQ ID NO: 24) core sequence of MBP^{MOOR}.

FIGS. 8A-8C shows flow cytometric screening of Gal transferases for biosynthesis of T antigen. FIG. 8A is a schematic of a flow cytometric screen designed to evaluate candidate Gal transferases (GalTs) for their ability to generate lipid-linked T antigen. Once formed, the T antigen is subsequently flipped to periplasm by the native E. coli flippase, Wzx, transferred to lipid A core by the promiscuous O-antigen ligase WaaL native to E. coli, and ultimately displayed on the cell surface. Cells are labeled with FITC-conjugated PNA that specifically binds the T antigen. FIG. 8B shows flow cytometric analysis of PNA-labeled E. coli MC4100 ΔwecA (MCΔw) (yellow) or MC4100 ΔwecA ΔwaaL (MCΔww) (gray) carrying no plasmid, plasmid pOG-Tn, or plasmid pOG-Tn modified with one of the candidate GalT enzymes as indicated. FIG. 8C shows flow cytometric analysis of PNA-labeled MCΔw (yellow) or MCΔww (gray) carrying no plasmid, plasmid pOG-T (producing T antigen glycan with EcWbwC), or plasmid pOG-TΔgne (encoding T antigen pathway but lacking CjGne epimerase). In FIG. 8B and FIG. 8C, unlabeled MCΔw cells (white) were included as negative controls. Inset histograms show representative flow cytometric data used to generate mean fluorescence Intensity data. Sec FIG. 6 for flow cytometry gating strategy.

FIG. 9 shows MS/MS fragmentation analysis of T-modified glycoprotein. EThcD fragmentation analysis of glycosylated peptide ³⁹⁷NVGGDLDWPAAAS(HexHexNAc)APQPGKPPR⁴¹⁸ (SEQ ID NO: 24) derived from MBP^{MOOR} by trypsin digestion. The spectrum identifies the neutral loss pattern of the HexHexNAc disaccharide, corresponding oxonium ions, and fragments of the glycopeptide (c and z ions), validating the glycosylation and the site of glycosylation at S409 within the 8-residue WPAAASAP (SEQ ID NO: 25) core sequence of MBP^{MOOR}.

FIGS. 10A-10B show orthogonal biosynthesis of sialylated O-glycoforms in E. coli. FIG. 10A shows nano-LC-MS/MS analysis of purified acceptor protein generated by nanA-deficient E. coli cells carrying plasmid pConNeuDBAC for CMP-NeuNAc biosynthesis along with pOG-T-NgPglO and pEXT-spDsbA-MBP^{MOOR}-EcWbwA. Sequence coverage of 88% was obtained for the MBP^{MOOR} protein in the analysis. Spectrum reveals a predominant species (80% abundance) corresponding to the indicated peptide fragment bearing a single HexHexNAc modification as well as three less abundant species bearing a single NeuNAcHexHexNAc, a single HexNAc, and no modification (16%, 2%, and 2%, respectively). FIG. 10B is the same as FIG. 10A, but with purified acceptor protein generated by nanA-deficient glyco-recoded cells carrying pOG-Tn-NgPglO and pEXT-spDsbA-MBP^{MOOR}-PspST6. Sequence coverage of 92% was obtained for MBP^{MOOR} in the analysis. Spectrum reveals a predominant species (90% abundance) corresponding to the indicated peptide fragment bearing a single HexNAc modification as well as two less abundant species bearing a single NeuNAcHexNAc and no modification (2% and 9%, respectively). Arrow denotes modified serine (bold underlined font) as determined by EThcD fragmentation analysis.

FIGS. 11A-11B are spectra showing MS/MS fragmentation analysis of ST- and STn-modified glycoproteins. EThcD fragmentation analysis of glycosylated peptide ³⁹⁷NVGGDLDWPAAAS(NeuNAcHexHexNAc)APQPGKPPR⁴¹⁸ (SEQ ID NO: 26) derived from ST-modified MBP^{MOOR} (FIG. 11A) and STn-modified MBP^{MOOR} (FIG. 11B) that were subjected to trypsin digestion. The spectrum identifies the neutral loss pattern of the single NeuNAc and Hex monosaccharides, corresponding oxonium ions, and fragments of the glycopeptide (c and z ions), validating the glycosylation and site of glycosylation at S409 within the 8-residue WPAAASAP (SEQ ID NO: 24) core sequence of MBP^{MOOR}.

FIGS. 12A-12B show yield determination for MBP^{MOOR} modified with different O-glycans. FIG. 12A is a coomassie-stained SDS-PAGE gel showing MBP^{MOOR} proteins purified from different strains. MBP^{MOOR} bearing Tn or T antigens was produced in CLM25 cells co-transformed with pEXT-based plasmid for acceptor protein and appropriate sialyltransferase expression and either pOG-Tn-NgPglO or pOG-T-NgPglO plasmids, respectively. MBP^{MOOR} bearing STn or ST antigens was produced in glyco-recoded cells carrying the CMP-NeuNAc biosynthesis pathway in the genome and co-transformed with pEXT-based plasmid for acceptor protein expression and either pOG-Tn-NgPglO or pOG-T-NgPglO plasmids, respectively. CLM25 cells co-transformed with only the pEXT-based plasmid for expressing MBP^{MOOR} (agly) and appropriate sialyltransferase served as the control. Molecular weight (M_{w}) marker included on the left. SDS-PAGE gel is representative of three biological replicates. FIG. 12B is a table showing the yield of each glycoprotein calculated by multiplying the total yield times the percentage glycosylated (% gly), the latter of which was determined from nano-LC-MS/MS analysis of each glycoprotein product. Yield values are the average of three biological replicates and the error is the standard deviation of the mean.

FIGS. 13A-13C show O-linked glycosylation of diverse protein targets. FIG. 13A shows the results of an immunoblot analysis of acceptor proteins purified from CLM25 cells co-transformed with pOG-T-NgPglO (+, top), pOG-T NmPglL (+, bottom), or pOG-T without an O-OST (−) along with pEXT-based plasmid encoding each of the different protein targets as indicated. MBP^{MOOR} and MBP^{MOORmut} derived from the same cells served as positive and negative control, respectively. Blots were probed with anti-hexa-histidine antibody (6×His) to detect acceptor proteins and PNA lectin to detect the T antigen. Molecular weight (M_{w}) markers are indicated on the left of each blot. All immunoblot results are representative of at least three biological replicates. FIGS. 13B-13C are the same as in FIG. 13A with pOG-T-NmPglL (+) or pOG-T without NmPglL (−) along with pEXT-based plasmid encoding each of the different protein targets as indicated.

FIG. 14 is an immunoblot showing secretion of O-glycoproteins in the culture supernatant. Immunoblot analysis of culture supernatants derived from CLM24 ΔyaiW cells co-transformed with pOG-T-NgPglO or pOG-T-NmPglL along with pEXT-based plasmid encoding YebF-MBP^{MOOR} or YebF-MBP^{MOORmut} as indicated. Mutation of acceptor serine to glycine in YebF-MBP^{MOORmut} served as negative control. Blots were probed with anti-hexa-histidine antibody (6×His) to detect acceptor proteins and PNA lectin to detect the T antigen. Molecular weight (M_{w}) markers are indicated on the left of each blot. Immunoblot results are representative of at least three biological replicates.

FIGS. 15A-15D show orthogonal biosynthesis of different MUC1 O-glycoforms in E. coli. Nano-LC-MS/MS analysis of purified acceptor protein generated by CLM25 cells carrying plasmid pOG-T-NgPglO along with pEXT-based plasmid for expression of different MUC1 constructs including: MUC1_8 (FIG. 15A); MUC1_20 (FIG. 15B); MUC1_24 (FIG. 15C); and MUC1_41 (FIG. 15D). Sequence coverage of 77% was obtained for MUC1_8, 78% for MUC1.20, 88% for MUC1.24, and 75% for MUC1_41 in the analysis. All spectra reveal a predominant species corresponding to the indicated peptide fragments bearing a single HexHexNAc modification. Additional less abundant species bearing a single HexNAc and no modification were observed in all cases. For MUC1_41, several doubly glycosylated species were also identified as minor species. Arrow denotes modified serine (bold underlined font) as determined by EThcD fragmentation analysis.

FIGS. 16A-16D show MS/MS fragmentation analysis of MUC1 O-glycoforms bearing the T antigen. EThcD fragmentation analysis of glycosylated peptides derived by trypsin digestion. The spectrum identifies the neutral loss pattern of HexHexNAc disaccharide, corresponding oxonium ions, and fragments of the glycopeptide (c and z ions), validating the glycosylation and the sites of glycosylation (S409 in MUC1_8; S415 in MUC1_20; S417 in MUC1_24 and S417 of MUC1_41) within relevant MUC1 peptides (SEQ ID NO: 47) as indicated in the inset sequences.

### DETAILED DESCRIPTION

### General Definitions

Unless otherwise indicated, the definitions and embodiments described in this and other sections are intended to be applicable to all embodiments and aspects of the present application herein described for which they are suitable as would be understood by a person skilled in the art.

As used herein, the singular forms “a,” “an,” and “the” and the like include plural referents unless the context clearly dictates otherwise. Thus, for example, reference to “a compound” includes both a single compound and a plurality of different compounds.

Terms of degree such as “substantially”, “about”, and “approximately” as used herein mean a reasonable amount of deviation of the modified term such that the end result is not significantly changed. These terms of degree should be construed as including a deviation of at least ±1% (and up to ±5% or ±10%) of the modified term if this deviation would not negate the meaning of the word it modifies.

The term “and/or” as used herein means that the listed items are present, or used, individually or in combination. In effect, this term means that “at least one of” or “one or more” of the listed items is used or present.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, and so on. As a non-limiting example, each range discussed herein can be readily broken down into a lower third, middle third and upper third, and so on. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member.

In understanding the scope of the present application, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “involving”, “having”, and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps. In embodiments or claims where the term comprising (or the like) is used as the transition phrase, such embodiments can also be envisioned with replacement of the term “comprising” with the terms “consisting of” or “consisting essentially of.” The methods, kits, systems, and/or compositions of the present disclosure can comprise, consist essentially of, or consist of, the components disclosed.

In embodiments comprising an “additional” or “second” component, the second component as used herein is different from the other components or first component. A “third” component is different from the other, first, and second components, and further enumerated or “additional” components are similarly different.

Certain terms employed in the specification, examples and claims are collected herein. Unless defined otherwise, all technical and scientific terms used in this disclosure have the same meanings as commonly understood by one of ordinary skill in the art to which this disclosure belongs.

Preferences and options for a given aspect, feature, embodiment, or parameter of the invention should, unless the context indicates otherwise, be regarded as having been disclosed in combination with any and all preferences and options for all other aspects, features, embodiments, and parameters of the invention.

As used herein, amino acid residues will be indicated either by their full name or according to the standard three-letter or one-letter amino acid code.

As used herein, the terms “polypeptide” or “protein” are used interchangeably, and refer to a polymeric form of amino acids of any length, which can include coded and non-coded amino acids, chemically or biochemically modified or derivatized amino acids, and polypeptides having modified peptide backbones. A “peptide” is also a polymer of amino acids with a length which is usually of up to 50 amino acids. A polypeptide or peptide is represented by an amino acid sequence.

As used herein, the terms “nucleic acid molecule”, “polynucleotide”, “polynucleic acid”, “nucleic acid” are used interchangeably and refer to polymeric form of nucleotides of any length, either deoxyribonucleotides or ribonucleotides, or analogs thereof. A nucleic acid molecule is represented by a nucleic acid sequence, which is primarily characterized by its base sequence. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term “homology” denotes at least secondary structural identity or similarity between two macromolecules, particularly between two polypeptides or polynucleotides, from same or different taxons, wherein said similarity is due to shared ancestry. Hence, the term “homologues” denotes so-related macromolecules having said secondary and optionally tertiary structural similarity. For comparing two or more nucleotide sequences, the “(percentage of) sequence identity” between a first nucleotide sequence and a second nucleotide sequence may be calculated using methods known by the person skilled in the art (e.g., by dividing the number of nucleotides in the first nucleotide sequence that are identical to the nucleotides at the corresponding positions in the second nucleotide sequence by the total number of nucleotides in the first nucleotide sequence and multiplying by 100% or by using a known computer algorithm for sequence alignment such as NCBI Blast). In determining the degree of sequence similarity between two amino acid sequences, the skilled person may take into account so-called “conservative” amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions have been already exemplified herein. Amino acid sequences and nucleic acid sequences are said to be “exactly the same” if they have 100% sequence identity over their entire length.

Throughout this disclosure, each time one refers to a specific amino acid sequence SEQ ID NO (take SEQ ID NO: Y as example), one may replace it by: a polypeptide comprising an amino acid sequence that has at least 80% sequence identity or similarity with amino acid sequence SEQ ID NO: Y. Throughout this application, the wording “a sequence is at least X % identical with another sequence” may be replaced by “a sequence has at least X % sequence identity with another sequence”.

Each amino acid sequence described herein by virtue of its identity percentage (at least 80%) with a given amino acid sequence respectively has in a further preferred embodiment an identity of at least 80%, 81%, 82%, 83%, 84%, 85%, 86%, 87%, 88%, 89%, 90%, 91%, 92%, 93%, 94%, 95%, 96%, 97%, 98%, 99% or more identity with the given amino acid sequence respectively. In some embodiments, sequence identity is determined by comparing the whole length of the sequences as identified herein. Each amino acid sequence described herein by virtue of its similarity percentage (at least 80%) with a given amino acid sequence respectively has in a further embodiment a similarity of at least 80%, 81%, 82%, 83%, 84%, 85%, 86%, 87%, 88%, 89%, 90%, 91%, 92%, 93%, 94%, 95%, 96%, 97%, 98%, 99% or more similarity with the given amino acid sequence respectively. In some embodiments, sequence similarity is determined by comparing the whole length of the sequences as identified herein. Unless otherwise indicated herein, identity or similarity with a given SEQ ID NO means identity or similarity based on the full length of said sequence (i.e., over its whole length or as a whole).

“Sequence identity” is herein defined as a relationship between two or more amino acid (polypeptide or protein) sequences or two or more nucleic acid (polynucleotide) sequences, as determined by comparing the sequences. The identity between two amino acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%.

In the art, “identity” also means the degree of sequence relatedness between amino acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M, and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, FASTA, BLASTN, and BLASTP (Altschul, S. F. et al., J. Mol. Biol. 215:403-410 (1990)), EMBOSS Needle (Madeira, F., et al., Nucleic Acids Research 47(W1): W636-W641 (2019)). The BLAST program is publicly available from NCBI and other sources (BLAST Manual, Altschul, S., et al., NCBI NLM NIH Bethesda, Md. 20894; Altschul, S., et al., J. Mol. Biol. 215;403-410 (1990)). The EMBOSS program is publicly available from EMBL-EBI. The well-known Smith Waterman algorithm may also be used to determine identity. The EMBOSS Needle program is the preferred program used.

Preferred parameters for polypeptide sequence comparison include the following: Algorithm: Needleman and Wunsch, J. Mol. Biol. 48 (3):443-453 (1970); Comparison matrix: BLOSUM62 from Henikoff and Henikoff, Proc. Natl. Acad. Sci. USA. 89:10915-10919 (1992); Gap Open Penalty: 10; and Gap Extend Penalty: 0.5. A program useful with these parameters is publicly available as the EMBOSS Needle program from EMBL-EBI. The aforementioned parameters are the default parameters for a Global Pairwise Sequence alignment of proteins (along with no penalty for end gaps).

Preferred parameters for nucleic acid comparison include the following: Algorithm: Needleman and Wunsch, J. Mol. Biol. 48:443-453 (1970): Comparison matrix: DNAfull; Gap Open Penalty: 10; Gap Extend Penalty: 0.5. A program useful with these parameters is publicly available as the EMBOSS Needle program from EMBL-EBI. The aforementioned parameters are the default parameters for a Global Pairwise Sequence alignment of nucleotide sequences (along with no penalty for end gaps).

Also provided herein are embodiments wherein any embodiment described herein may be combined with any one or more other embodiments, provided the combination is not mutually exclusive.

### Eukaryotic O-Glycosylation Pathways

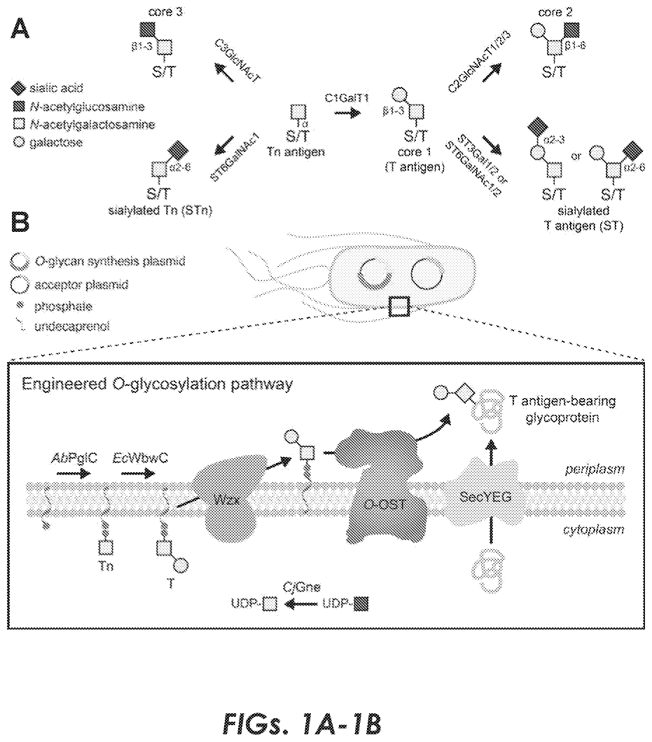

FIG. 1A is a schematic showing exemplary eukaryotic O-glycosylation pathways, which are coordinated by the activities of eukaryotic glycosyltransferases (GTs) ((Schwarz & Aebi, “Mechanisms and Principles of N-Linked Protein Glycosylation,” Curr. Opin. Struct. Biol. 21:576-582 (2011), which is hereby incorporated by reference in its entirety). As shown in FIG. 1A, mucin-type O-glycan synthesis originates from the hydroxyl group of a serine or threonine (S/T) amino acid by the addition of N-acetylgalactosamine (GalNAc) by N-acetylgalactosaminyl-transferase 2 (GalNAcT2) to form the Tn antigen stricture (GalNAcα-S/T). In one exemplary eukaryotic glycosylation pathway, core 3β1-3 N-acetylglucosaminyltransferase (C3GlcNAcT) adds ß1,3-linked N-acetylglucosamine (GlcNAc) to the initial Tn antigen (GalNAcα-S/T) (see pathway beginning in the middle of FIG. 1A and continuing to the upper left of FIG. 1A). In another exemplary eukaryotic glycosylation pathway, α2-6 sialyltransferase (ST6GalNAcl) adds α2-6-linked sialic acid to the initial Tn antigen (GalNAcα-S/T) to generate sialylated Tn (STn) (see pathway beginning in the middle of FIG. 1A and continuing to the lower left of FIG. 1A). In a further exemplary eukaryotic glycosylation pathway, core 1 synthase glycoprotein-N-acetylgalactosamine 3-β-galactosyltransferase (C1GalT1) adds β1,3-linked galactose (Gal) to the initial GalNAcα-S/T to generate the T antigen (see pathway beginning in the middle of FIG. 1A and continuing to the right of FIG. 1A). T antigen can be further elaborated with GlcNAc and NeuNAc in a variety of ways, as shown in FIG. 1A. For example, core 2 β1-6 N-acetylglucosaminyltransferase (C2GlcNAcT1/2/3) may add β1-6-linked N-acetylglucosamine to the T antigen (see pathway beginning in the middle of FIG. 1A and continuing to the upper right of FIG. 1A); core 1 α2-3 sialyltransferase (ST3Gal1/2) may add α2-3-linked sialic acid to T antigen to generate sialylated T antigen (ST) (see pathway beginning in the middle of FIG. 1A and continuing to the bottom right of FIG. 1A); or α2-6 sialyltransferase (ST6GalNAc1/2) may add α2-6-linked sialic acid to T antigen to generated sialylated T antigen (ST) (see pathway beginning in the middle of FIG. 1A and continuing to the bottom right of FIG. 1A).

As described herein above, glycoengineering in eukaryotes is complicated by the fact that glycans are synthesized across several subcellular compartments and that glycosylation is an essential process, with significant alteration of glycosylation pathways often leading to severe fitness defects (Choi et al., “Use of Combinatorial Genetic Libraries to Humanize N-Linked Glycosylation in the Yeast Pichia pastoris,” Proc. Natl. Acad. Sci. USA 100:5022-5027 (2003), which is hereby incorporated by reference in its entirety). Glycoengineering in bacteria, on the other hand, is not constrained by these issues due to the non-essential nature of protein glycosylation in bacterial cells.

The present disclosure provides recombinant prokaryotic host cells, as well as lysates of such recombinant prokaryotic host cells and related kits, devices, compositions, systems, and methods for producing O-glycosylated proteins. Specifically, the present disclosure provides for the development of a low-cost strategy for efficient production of O-linked glycoproteins in prokaryotic host cells (or using lysates thereof). In some embodiments, the recombinant prokaryotic host cells of the present disclosure have been genetically engineered with a one or more genes encoding a novel O-glycosylation pathway that is capable of efficiently glycosylating target proteins at specific acceptor sites (e.g., O-linked glycosylation). Using these engineered recombinant prokaryotic host cells, virtually any recombinant protein-of-interest can be expressed and glycosylated.

### Recombinant Prokaryotic Host Cells

A first aspect of the present disclosure relates to a recombinant prokaryotic host cell expressing one or more 4-epimerases, one or more glycosyl-1-phosphate transferases, and one or more O-oligosaccharyltransferases.

Another aspect of the present disclosure relates to a recombinant prokaryotic host cell expressing one or more 4-epimerases, one or more glycosyl-1-phosphate transferases, one or more O-oligosaccharyltransferases, and one or more ß1,3-galactosyltransferase enzymes. In some embodiments, the one or more ß1,3-galactosyltransferase enzymes are capable of transferring galactose (Gal) to undecaprenyl pyrophosphate (Und-PP)-linked N-Acetylgalactosamine (GalNAc).

Another aspect of the present disclosure relates to a prokaryotic host cell expressing an α2,6-sialyltransferases and an α2,3-sialyltransferase, where the α2,6-sialyltransferases is the α2,6-sialyltransferases from Photobacterium sp. JT-ISH-224 (PspST6) and where the α2,3-sialyltransferase is the α2,3-sialyltransferase from E. coli O104 (EcWbwA).

Another aspect of the present disclosure relates to a prokaryotic host cell expressing one or more 4-epimerases, one or more glycosyl-1-phosphate transferases, one or more O-Antigen ligases (e.g., EcWaaL), and optionally one or more ß1,3-galactosyltransferase enzymes capable of transferring galactose (Gal) to undecaprenyl pyrophosphate (Und-PP)-linked N-Acetylgalactosamine (GalNAc). In some embodiments according to this aspect of the invention, the prokaryotic host cell does not encode an O-oligosaccharyltransferase.

Recombinant prokaryotic cells according to the present disclosure serve as a host for expression of recombinant proteins for production of O-glycosylated proteins of interest. Suitable host cells include, without limitation, E. coli and other Enterobacteriaceae, Escherichia sp., Campylobacter sp., Wolinella sp., Desulfovibrio sp. Vibrio sp., Pseudomonas sp. Bacillus sp., Listeria sp., Staphylococcus sp., Streptococcus sp., Peptostreptococcus sp., Megasphaera sp., Pectinatus sp., Selenomonas sp., Zymophilus sp., Actinomyces sp., Arthrobacter sp., Frankia sp., Micromonospora sp., Nocardia sp., Propionibacterium sp., Streptomyces sp., Lactobacillus sp., Lactococcus sp., Leuconostoc sp., Pediococcus sp., Acetobacterium sp., Eubacterium sp., Heliobacterium sp., Heliospirillum sp., Sporomusa sp., Spiroplasma sp., Mycoplasma sp., Erysipelothrix sp., Corynebacterium sp. Enterococcus sp., Clostridium sp., Mycoplasma sp., Mycobacterium sp., Actinobacteria sp., Salmonella sp., Shigella sp., Moraxella sp., Helicobacter sp., Stenotrophomonas sp., Micrococcus sp., Neisseria sp., Bdellovibrio sp., Hemophilus sp., Klebsiella sp., Proteus mirabilis, Enterobacter cloacae, Serratia sp., Citrobacter sp., Proteus sp., Serratia sp., Yersinia sp., Acinetobacter sp., Actinobacillus sp. Bordetella sp., Brucella sp., Capnocytophaga sp., Cardiobacterium sp., Eikenella sp., Francisella sp., Haemophilus sp., Kingella sp., Pasteurella sp., Flavobacterium sp. Xanthomonas sp., Burkholderia sp., Aeromonas sp., Plesiomonas sp., Legionella sp., Rhizobium sp., and Azototoacter sp. (e.g., A. vinelandii).

One major advantage of E. coli as a prokaryotic host cell for O-glycoprotein expression is that, unlike yeast and all other eukaryotes, there are no native glycosylation systems. Thus, the addition (or subsequent removal) of glycosylation-related genes should have little to no bearing on the viability of glyco-engineered E. coli cells. Furthermore, the potential for non-human glycan attachment to target proteins by endogenous glycosylation reactions is eliminated in these cells. Accordingly, in various embodiments, a prokaryotic host cell (or lysate thereof) is used to produce O-linked glycoproteins, which provides an attractive solution for circumventing the significant hurdles associated with eukaryotic cell culture. Suitable E. coli host cells according to the present disclosure include, without limitation, laboratory strains of E. coli selected from the group consisting of DH5α, NEB 10-beta, BL21(DE3), W3110, CLM24, CLM25, MC4100, MCΔw, MCΔΔw, MCΔΔw-neuo-PS, MCΔΔwΔn-neuo-PS, and ZLKA (see, e.g., Table 5, infra).

In some embodiments of the present disclosure, the one or more 4-epimerases, the one or more glycosyl-1-phosphate transferases, the one or more O-oligosaccharyltransferases, and/or the one or more ß1,3-galactosyltransferase enzymes are orthogonal and/or heterologous to the prokaryotic host cell. The term “orthogonal” refers to a molecule (e.g., an enzyme) that functions with endogenous components of a host cell with reduced efficiency as compared to a corresponding molecule that is endogenous to the host cell, or that fails to function with endogenous components of the cell. In some embodiments, the orthogonal enzyme lacks a functionally normal endogenous complementary enzyme in the host cell. A second orthogonal enzyme can be introduced into the cell that functions with the first orthogonal enzyme. For example, an orthogonal O-glycosylation pathway may include introduced complementary components that function together in the host cell (e.g., one or more 4-epimerases, the one or more glycosyl-1-phosphate transferases, the one or more O-oligosaccharyltransferases, and the one or more ß1,3-galactosyltransferase enzymes).

The term “heterologous” refers to a molecule (e.g., an enzyme or a nucleic acid sequence encoding an enzyme) not normally found in the host organism. The term “heterologous” also includes a nucleic acid molecule comprising a native coding region, or portion thereof, that is reintroduced into the host organism in a form that is different from the corresponding native gene (e.g., not in its natural location in the host cell genome or in a codon-optimized format). Thus, in some embodiments, the one or more 4-epimerases, the one or more glycosyl-1-phosphate transferases, the one or more O-oligosaccharyltransferases, and/or the one or more ß1,3-galactosyltransferase enzymes are heterologous to the prokaryotic host cell.

In some embodiments, the 4-epimerase is a uridine diphosphate-N-acetylglucosamine (UDP-GlcNAc) 4 epimerase. As used herein, the term “UDP-GlcNAc 4-epimerase” refers to an enzyme that catalyzes the epimerization of the hydroxyl group at position C-4 of UDP-GlcNAc (uridine diphosphate-N-acetylglucosamine) to generate uridine diphosphate-N-acetylgalactosamine (UDP-GalNAc) (see, e.g., Bernatchez et al., “A Single Bifunctional UDP-GlcNAc/Glc 4-Epimerase Supports the Synthesis of Three Cell Surface Glycoconjugates in Campylobacter jejuni,” J. Biol. Chem. 280(6):P4792-4802 (2005), which is hereby incorporated by reference in its entirety). Thus, in some embodiments, the one or more 4-epimerases comprises a UDP-GlcNAc 4-epimerase (Gne). Suitable UDP-GlcNAc 4-epimerases include, without limitation, C. jejuni Gne (CjGne), Salmonella enterica O30 Gne (SeGne), Shigella boydii 018 Gne (SbGne), E. coli 055 Gne (EcGne), E. coli 086 Gne (EcGne), E. coli 086 Gne2 (EcGne2). In other embodiments, the one or more 4-epimerases belongs to the KEGG orthology ID KO01784 (see, e.g., Meceratesi et al., “Human UDP-Galactose 4′ Epimerase (GALE) Gene and Identification of Five Missense Mutations in Patients with Epimerase-Deficiency Galactosemia,” Mol. Genet. Metab. 63:26-30 (1998) and Majumdar et al., “UDPgalactose 4-Epimerase from Saccharomyces cerevisiae. A Bifunctional Enzyme with Aldose 1-Epimerase activity,” Eur. J. Biochem. 271:753-759 (2004), which are hereby incorporated by reference in their entirety). Suitable 4-epimerases belonging to KEGG orthology ID KO01784 include, without limitation, UDP-galactose-4-epimerases (GALE) enzymes.

The amino acid sequence of C. jejuni Gne (CjGne) has the amino acid sequence of SEQ ID NO: 1 below.

(SEQ ID NO: 1)
MKILISGGAGYIGSHTLRQFLKTDHEICVLDN
LSKGSKIAIEDLQKIRTFKFFEQDLSDFQGVK
ALFEREKFDAIVHFAASIEVFESMQNPLKYYM
NKTVNTTNLIETCLQTGVNKFIFSSTAATYGE
PQTPVVSETSPLAPINPYGRSKLMSEEVLRDA
SMANPEFKHCILRYFNVAGACMDYTLGQRYPK
ATLLIKVAAECAAGKRNKLFIFGDDYDTKDGT
CIRDFIHVDDISSAHLSALDYLKENESNVFNV
GYGHGFSVKEVIEAMKKVSGVDFKVELAPRRA
GDPSVLISDASKIRNLTSWQPKYDDLGLICKS
AFDWEKQCL

Additional exemplary UDP-GlcNAc 4-epimerases include, without limitation. Pseudomonas aeruginosa WbpP and Plesiomonas shigelloides WbgU (see, e.g., Ishiyama et al., “Crystal Structure of WbpP, a Genuine UDP-N-Acetylglucosamine 4-Epimerase from Pseudomonas aeruginosa: Substrate Specificity in Udp-Hexose 4-Epimerases,” J. Biol. Chem. 279(21):22635-22642 (2004) and Kowal & Wang et al., “New UDP-GlcNAc C4 Epimerase Involved in the Biosynthesis of 2-Acetamino-2-deoxy-L-Altruronic Acid in the O-Antigen Repeating Units of Plesiomonas shigelloides O17,” Biochemistry 41(51):15410-15414 (2002), which is hereby incorporated by reference in its entirety).

As used herein, the term “glycosyl-1-phosphate transferase” refers to an enzyme that transfers a phosphate-monosaccharide from a nucleotide diphosphate-monosaccharide to a polyprenol phosphate to generate a polyprenol diphosphate-linked monosaccharide.

In some embodiments, the glycosyl-1-phosphate transferase transfers N-acetylgalactosamine (GalNac) from UDP-GalNAc to undecaprenol phosphate (Und-P) to form undecaprenol pyrophosphate (Und-PP)-linked GalNAc. Suitable glycosyl-1-phosphate transferases include, without limitation, PglC. In some embodiments, the PglC is an Acinetobacter baumannii PglC (AbPglC).

Suitable Acinetobacter baumannii PglC (AbPglC) enzymes may be selected from the group consisting of Acinetobacter baumannii strain ATCC 17978 PglC, Acinetobacter baumannii strain NIPH190, Acinetobacter baumannii strain D46, Acinetobacter baumannii strain LUH5541, Acinetobacter baumannii LUH5546, Acinetobacter baumannii RBH4, Acinetobacter baumannii strain A74, Acinetobacter baumannii strain ACICU, Acinetobacter baumannii strain LUH5533, Acinetobacter baumannii strain LUH5550, Acinetobacter baumannii strain NIPH70, and Acinetobacter baumannii strain 4190 (Harding et al., “Distinct Amino Acid Residues Confer One of Three UDP-Sugar Substrate Specificities in Acinetobacter baumannii PglC Phosphoglycosyltransferases,” Glycobiology 28(7):522-533 (2018), which is hereby incorporated by reference in its entirety).

The amino acid sequence of Acinetobacter baumannii strain ATCC 17978 PglC has the amino acid sequence of SEQ ID NO: 2 below.

(SEQ ID NO: 2)
MVNENMKRLVDIVISLIALTVLSPIFLIVAYK
VRKNLGSPIFFYQERPGKDGKLFKMIKFRSMK
DAFDAQGNPLPDEARITPFGQKLRSTSLDEMP
QLINVLKGDMSVVGPRPMLKDFVALYSPEQAR
RLEVRPGMTGLAQVSGRNELDYEERFKCDVWY
VDNHNIWVDFKIMFKTVKVMLKREGINAPGHV
GPSLFKGNDTQENIDSSVK

As used herein, the term “ß1,3-galactosyltransferase” refers to an enzyme that transfers galactose (Gal) to a polyprenol diphosphate-linked monosaccharide (e.g., undecaprenol pyrophosphate (Und-PP)-linked GalNAc).

In some embodiments, the one or more ß1,3-galactosyltransferase enzymes capable of transferring galactose (Gal) to undecaprenyl pyrophosphate (Und-PP)-linked N-Acetylgalactosamine (GalNAc) is a ß-1,3; galactosyltransferase derived from the O-antigen biosynthesis pathway of an enterohemorrhagic Escherichia coli (EcWbwC). Suitable ß-1,3; galactosyltransferases derived from the O-antigen biosynthesis pathway of an enterohemorrhagic Escherichia coli include, without limitation, E. coli strain O104 EcWbwC and E. coli strain O5 EcWbwC.

As described herein, E. coli strain O104 encodes the ß-1,3,-galactosyltransferase WbwC, which extends Und-PP-GalNAc by a single Gal residue, yielding lipid-linked Gal-β1,3-GalNAc. The Gal-β1,3-GalNAc disaccharide present on E. coli strain O104 O antigens is identical to the O-glycan core 1 of mammalian glycoproteins and the cancer-associated Thomsen-Friedenreich (TF or T) antigen (see, e.g., Wang et al., “Characterization of Two UDP-Gal:GalNAc-Diphosphate-Lipid β1,3-Galactosyltransferases WbwC from Escherichia coli Serotypes O104 and O5,” J. Bacteriol. 196(17):3122-3133 (2014), which is hereby incorporated by reference in its entirety).

The amino acid sequence of E. coli strain O104 EcWbwC has the amino acid sequence of SEQ ID NO: 3 below.

(SEQ ID NO: 3)
MKFSVLLSLYYKESALFLHDCLESIASNTCAP
DQIVIVFDGYISDDLLHVVNEFSLRLPIDIVR
LSNNVGLGKALNHGLQYCRNELIFRMDTDDIC
LPERFALQLSYMKSHPEVVLLGSAIEBFDNTM
KIRQGKRFSVIYHEDIKKFAKKRNPFNHMTVV
FRKSVIEKLGGYQHHYLMEDYNLWLRILAADY
RTHNLSEVLVNVRAGRNMLCRRKGYSYIKSEI
LLAKLKYELQLDNLTGVIYTGVIRIIPRILPV
SLLKLVYNILRK

As used herein, the term “O-oligosaccharyltransferase” refers to an enzyme that transfers an O-oligosaccharide from a lipid carrier molecule to an acceptor molecule (e.g., the hydroxyl group of a serine or threonine residue on a target protein). In some embodiments, the O-oligosaccharyltransferase described herein glycosylates the acceptor molecule via an en bloc mechanism.

The one or more O-oligosaccharyltransferases (O-OST) may be PglL, PglO, or a combination thereof.

O-glycosylation of proteins in Neisseria meningitidis is catalyzed by PglL, which belongs to a protein family including WaaL O-antigen ligases. Neisseria meningitides PglL (NmPglL) shows relaxed substrate specificity and is able to transfer O-oligosaccharides composed of different sugars, linkages, and lengths from an undecaprenyl pyrophosphate (Und-PP) carrier to proteins (Faridmoayer et al., “Extreme Substrate Promiscuity of the Neisseria Oligosaccharyl Transferase Involved in Protein O-glycosylation,” J. Biol. Chem. 283(50):34596-604 (2008) and Faridmoayer et al., “Functional Characterization of Bacterial Oligosaccharyl-Transferases Involved in O-linked Protein Glycosylation,” J. Bacteriol. 189(22):8088-8098 (2007), which are hereby incorporated by reference in their entirety). Neisseria gonorrhoeae PglO (NgPglO) is an O-oligosaccharyltransferases having 95% sequence identity with Neisseria meningitides PglL (NmPglL) which glycosylates a wide range of periplasmic proteins containing serine and threonine residues in vivo (Hartley et al., “Biochemical Characterization of the O-Linked Glycosylation Pathway in Neisseria gonorrhoeae Responsible for Biosynthesis of Protein Glycans Containing N,N′-Diacetylbacillosamine,” Biochemistry 50(22):4936-4948 (2011), which is hereby incorporated by reference in its entirety).

In some embodiments, the PglL is a Neisseria meningitides PglL (NmPglL) and the PglO is Neisseria gonorrhoeae PglO (NgPglO).

The amino acid sequence of Neisseria meningitides PglL (NmPglL) has the amino acid sequence corresponding to nucleic acid residues 1-615 of SEQ ID NO: 4 below.

(SEQ ID NO: 4)
PAETTVSGAHPAAKLPIYILPCFLWIGIVPFTFALKLKPSPDFYHDAAA
AAGLIVLLFLTAGKKLFDVKIPAISFLLFAMAAFWYLQARLMNLIYPGM
NDIVSWIFILLAVSAWACRSLVAHFGQERIVTLFAWSLLIGSLLQSCIV
VIQFAGWEDTPLFQNIIVYSGQGVIGHIGQRNNLGHYLMWGILAAAYLN
GQRKIPAALGVICLIMQTAVLGLVNSRTILTYIAAIALILPFWYFRSDK
SNRRTMLGIAAAVFLTALFQFSMNTILETFTGIRYETAVERVANGGFTD
LPRQIEWNKALAAFQSAPIFGHGWNSFAQQTFLINAEQHNIYDNLLSNL
FTHSHNIVLQLLAEMGISGTLLVAATLLTGIAGLLKRPLTPASLFLICT
LAVSMCHSMLEYPLWYVYFLIPFGLMLFLSPAEASDGIAFKKAANLGIL
TASAAIFAGLLHLDWTYTRLVNAFSPATDDSAKTLNRKINELRYISANS
PMLSFYADFSLVNFALPEYPETQTWAEEATLKSLKYRPHSATYRIALYL
MRQGKVAEAKQWMRATQSYYPYLMPRYADEIRKLPVWAPLLPELLKDCK
AFAAAPGHPEAKPCKGSGYPYDVPDYAHHHHHHHHHH

The amino acid sequence of Neisseria gonorrhoeae PglO (NgPglO) has the amino acid sequence corresponding to amino acid residues 1-613 SEQ ID NO: 5 below.

(SEQ ID NO: 5)
MSAETTVSGARPAAKLPIYILPCFLWIGIIPFTFALRLKPSPDFYHDAAA
AAGLIVLLFLTAGKKLFDVKIPAISFLLFAMAAFWWLQARLMNLIYPGMN
DIASWVFILLAVSAWACKSLVAHYGQERIVTLFAWSLLIGSLLQSCIVVI
QFAGWENTPLLQNIIVHRGQGVIGHIGQRNNLGHYLMWGILASAYLNGQR
KIPAALGAICLIMQTAVLGLVNSRTILTYIAAIALILPFWYFRSDKSNRR
TMLGIAAAVFLTALFQFSMNAILETFTGIRYETAVERVANGGFTDLPRQS
EWNKALAAFQSAPIFGHGWNSFAQQTFLINAEQHTIHDNFLSTLFTHSHN
IILQLLAEMGLSGTLLVAATLLTGIAGLLKRSLTPASLFLLCALAVSMCH
SMLEYPLWYVYFLIPFGLMLFLSPAEASDGIAFKKAANLGILTASAAIFA
GLLHLDWTYTRLVNSFSPAADDSAKTLNRKINELRYISANSPMLSFYADF
SLVNFALPEYPETQTWAEEATLKALKYRPYSATYRIALYLMRQGKVAEAK
QWMRATQSYYPYLMPRYADEIRKLPVWAPLLPELLKDCKAFAAAPGHPET
KPCKYPYDVPDYAHHHHHHHHHH

Additional O-oligosaccharyltransferases are well known to those of skill in the art and include, without limitation proteins comprising protein glycosylation ligase (PglL_A), O-antigen ligase (Wzy_C), and/or virulence factor membrane-bound polymerase, C-terminal (Wzy_C_2) domains (see, e.g., Musumeci et al., “Evaluating the Role of Conserved Amino Acids in Bacterial O-Oligosaccharyltransferases by In Vivo, In Vitro and Limited Proteolysis Assays,” Glycobiology 24(1):39-50 (2014): Klena et al., “Comparison of Lipopolysaccharide Biosynthesis Genes rfaK, rfaL, rfaY, and rfaZ of Escherichia coli K-12 and Salmonella typhimurium,” J. Bacteriol. 174(14):4746-4752 (1992); Kadioglu et al., “The Role of Streptococcus pneumoniae Virulence Factors in Host Respiratory Colonization and Disease,” Nat. Rev. Microbiol. 6(4):288-301 (2008), which are hereby incorporated by reference in their entirety).

An exemplary embodiment of the present disclosure is shown schematically in FIG. 1B. In this embodiment, a 4-epinerase (e.g., Campylobacter jejuni uridine diphosphate-N-acetylglucosamine (UDP-GlcNAc) 4-epimerase (CjGne)) maintains a pool of uridine diphosphate-N-acetylgalactosamine (UDP-GalNAc) that serves as the activated nucleotide sugar donor for a glycosyl-1-phosphate transferase (e.g., Acinetobacter baumannii ATCC 17978 PglC (AbPglC)), which catalyzes the formation of undecaprenol pyrophosphate (Und-PP)-linked GalNAc. Next, a ß1,3-galactosyltransferase (e.g., Escherichia coli O104 WbwC (EcWbwC)) extends Und-PP-GalNAc by a single galactose (Gal) residue, yielding lipid-linked Gal-β1,3-GalNAc. Following flipping of the lipid-linked oligosaccharide (LLO) to the periplasmic face of the cytoplasmic membrane by a flippase (e.g., the native E. coli flippase Wzx), the preassembled T antigen glycan is transferred en bloc to the hydroxyl group of a serine or threonine amino acid on a Sec pathway-exported acceptor protein by an D-Oligosaccharyltransferase (O-OST) such as Neisseria gonorrhoeae PglO (NgPglO) or Neisseria meningitides PglL (NmPglL). It is noted that the absence of the ß1,3-galactosyltransferase (e.g., EcWbwC) enables generation of Tn-modified acceptor proteins (see FIG. 1A) while the further elaboration of Gal-ß1,3-GalNAc with additional sugars such as NeuNAc (see FIG. 1A) followed by transfer to protein is also possible.

As described herein, undecaprenyl-phosphate alpha-N-acetylglucosaminyl 1-phosphate transferase enzymes catalyze the transfer of a GlcNAc-1-phophate moiety (see FIG. 1B) from UDP-GlcNAc to form an undecaprenol pyrophosphate (Und-PP)-linked GlcNAc. Thus, undecaprenyl-phosphate alpha-N-acetylglucosaminyl 1-phosphate transferase activity may interfere with the exemplary engineered O-glycosylation pathway of FIG. 1B, which requires that uridine diphosphate-N-acetylgalactosamine (UDP-GalNAc) be available as a donor for the glycosyl-1-phosphate transferase (e.g., Acinetobacter baumannii ATCC 17978 PglC (AbPglC)). Accordingly, in some embodiments, the recombinant prokaryotic host cell does not express an enzymatically active undecaprenyl-phosphate alpha-N-acetylglucosaminyl 1-phosphate transferase. Exemplary undecaprenyl-phosphate alpha-N-acetylglucosaminyl 1-phosphate transferases include, without limitation, E. coli WecA. Thus, in some embodiments, the recombinant prokaryotic host cell is an E. coli host cell that lacks a functional copy of the wecA gene.

As described herein supra, O-glycosylation of proteins in Neisseria meningitides is catalyzed by PglL, which belongs to a protein family including WaaL O-antigen ligases. WaaL O-antigen ligases are inner membrane glycosyltransferases that catalyze the transfer of O-antigen polysaccharide from a lipid-linked intermediate to a terminal sugar of the lipid A-core oligosaccharide, which is a conserved step in lipopolysaccharide biosynthesis (Ruan et al., “Escherichia coli and Pseudomonas aeruginosa Lipopolysaccharide O-antigen Ligases share Similar Membrane Topology and Biochemical Properties,” Mol. Biol. 110(1):95-113 (2018), which is hereby incorporated by reference in its entirety).

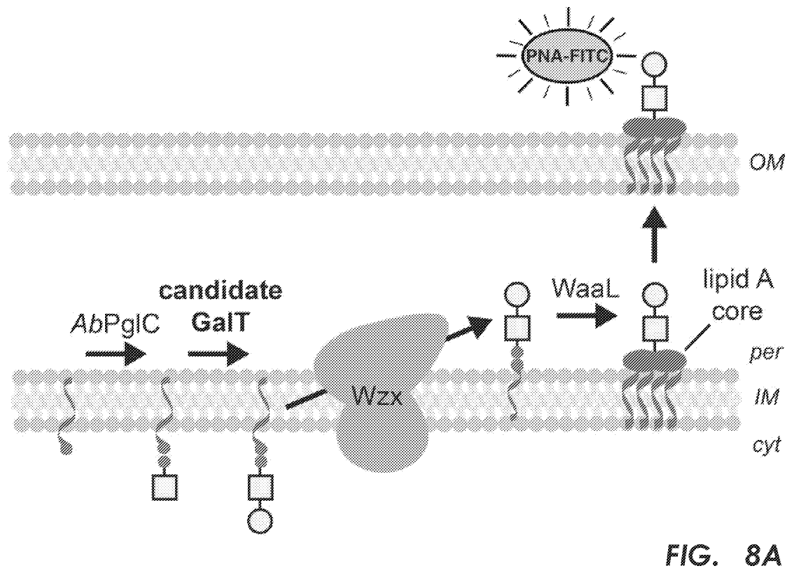

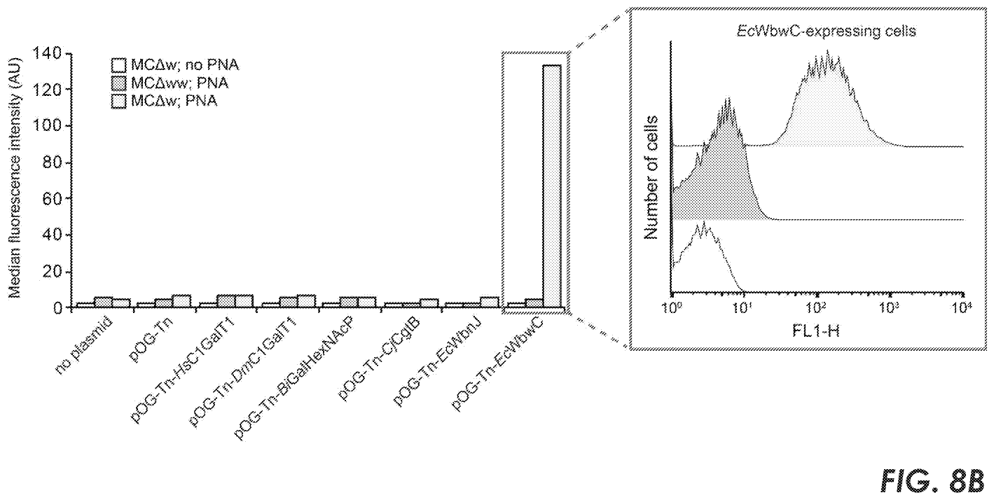

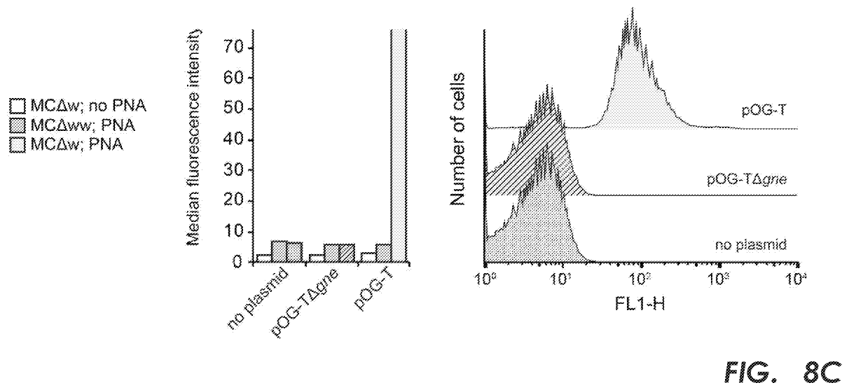

FIG. 8A is a schematic showing the transfer of an O-antigen polysaccharide from a lipid-linked intermediate to the lipid A-core oligosaccharide. More specifically, FIG. 8A illustrates the formation of undecaprenol pyrophosphate (Und-PP)-linked GalNAc by the glycosyl-1-phosphate transferase Acinetobacter baumannii ATCC 17978 PglC (AbPglC). Next, a candidate galactosyltransferase (GalT) is screened for its ability to extend Und-PP-GalNAc by a single galactose (Gal) residue to produce a lipid-linked Gal-GalNAc (T antigen). Once formed, the T antigen is flipped to periplasm by a native flippase (e.g., E. coli flippase Wzx) and transferred to a lipid A core by the promiscuous O-antigen ligase WaaL native to E. coli. Thus, the E. coli O-antigen ligase WaaL may interfere with the exemplary engineered O-glycosylation pathway of FIG. 1B, which involves the transfer of the preassembled T antigen glycan to the hydroxyl group of a serine or threonine amino acid on a Sec pathway-exported acceptor protein by an O-Oligosaccharyltransferase (O-OST) such as Neisseria gonorrhoeae PglO (NgPglO) or Neisseria meningitides PglL (NmPglL). Accordingly, in some embodiments, the recombinant prokaryotic host cell does not express an enzymatically active O-antigen ligase. Exemplary O-antigen ligases include, without limitation, E. coli WaaL. Thus, in some embodiments, the recombinant prokaryotic host cell is an E. coli host cell that lacks a functional copy of the waaL gene.

Recombinant prokaryotic host cells according to the present disclosure may be obtained by providing one or more nucleotide sequences encoding an enzyme of the present disclosure (e.g., the nucleotide sequences encoding the one or more 4-epimerases, the one or more glycosyl-1-phosphate transferases, the one or more O-oligosaccharyltransferases, the one or more ß1,3-galactosyltransferase enzymes, the enzymes of an enzymatic pathway capable of producing cytidine-5′-monophosphate-5-N-acetylneuraminic acid (CMP-NeuNAc), and/or the sialyltransferases of the present disclosure). Thus, each of the one or more 4-epimerases, the one or more glycosyl-1-phosphate transferases, the one or more O-oligosaccharyltransferases, the one or more ß1,3-galactosyltransferase enzymes, the enzymes of an enzymatic pathway capable of producing cytidine-5′-monophosphate-5-N-acetylneuraminic acid (CMP-NeuNAc), and/or the sialyltransferases of the present disclosure may be encoded by a nucleotide sequence that is independently located either on an extrachromosomal plasmid carried by the prokaryotic host cell or in the recombinant prokaryotic host cell's genome.

In some embodiments, the one or more nucleotide sequences encoding the one or more 4-epimerases, the one or more glycosyl-1-phosphate transferases, the one or more O-oligosaccharyltransferases, the one or more ß1,3-galactosyltransferase enzymes, the enzymes of an enzymatic pathway capable of producing cytidine-5′-monophosphate-5-N-acetylneuraminic acid (CMP-NeuNAc), and/or the sialyltransferases of the present disclosure is a recombinant genetic construct.

As used herein, the “recombinant genetic construct” of the disclosure refers to a nucleic acid molecule containing a combination of two or more genetic elements not naturally occurring together. The recombinant genetic construct may comprise non-naturally occurring nucleotide sequences that can be in the form of linear DNA, circular DNA, i.e., placed within a vector (e.g., a bacterial vector) or integrated into a genome.

As described in more detail infra, the recombinant genetic construct is introduced into the host cell of interest to effectuate the expression of the one or more 4-epimerases, the one or more glycosyl-1-phosphate transferases, the one or more O-oligosaccharyltransferases, the one or more ß1,3-galactosyltransferase enzymes, the enzymes of an enzymatic pathway capable of producing cytidine-5′-monophosphate-5-N-acetylneuraminic acid (CMP-NeuNAc), and/or the sialyltransferases, as disclosed herein.

Suitable nucleotide sequences encoding the one or more 4-epimerases, the one or more glycosyl-1-phosphate transferases, the one or more O-oligosaccharyltransferases, and/or the one or more ß1,3-galactosyltransferase enzymes disclosed herein are set forth in Table 1 below. Suitable nucleotide sequences also include nucleotide sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the 4-epimerase, the glycosyl-1-phosphate transferase, the O-oligosaccharyltransferase, and the ß1,3-galactosyltransferase coding sequences provided in Table 1 below (i.e., SEQ ID NOs. 6-10)

TABLE 1
Suitable 4-Epimerase, Glycosyl-1-Phosphate
Transferase, the O-Oligosaccharyltransferase,
and β1,3-Galactosyltransferase Coding Sequences

| | | SEQ |
|---|---|---|
| Name | Sequence | ID NO. |
| C. jejuni Gne | ATGAAAATTCTTATTAGCGGTGGTGCAGGTTATATAGGTTCTCATACTTTAAG | 6 |
| (CjGne) | ACAATTTTTAAAAACAGATCATGAAATTTGTGTTTTAGATAATCTTTCTAAGG | |
| | GTTCTAAAATCGCAATAGAAGATTTGCAAAAAATAAGAACTTTTAAATTTTTT | |
| | GAACAAGATTTAAGTGATTTTCAAGGCGTAAAAGCATTGTTTGAGAGAGAAAA | |
| | ATTTGACGCTATTGTGCATTTTGCAGCGAGCATTGAAGTTTTTGAAAGTATGC | |
| | AAAACCCTTTAAAGTATTATATGAATAACACTGTTAATACGACAAATCTCATC | |
| | GAAACTTGTTTGCAAACTGGAGTGAATAAATTTATATTTTCTTCAACGGCAGC | |
| | CACTTATGGCGAACCACAAACTCCCGTTGTGAGCGAAACAAGTCCTTTAGCAC | |
| | CTATTAATCCTTATGGGCGTAGTAAGCTTATGAGCGAAGAGGTTTTGCGTGAT | |
| | GCAAGTATGGCAAATCCTGAATTTAAGCATTGTATTTTAAGATATTTTAATGT | |
| | TOCAGGTGCTTGCATGGATTATACTTTAGGACAACGCTATCCAAAAGCGACTT | |
| | TGCTTATAAAAGTTGCAGCTGAATGTGCCGCAGGAAAACGTAATAAACTTTTC | |
| | ATATTTGGCGATGATTATGATACAAAAGATGGCACTTGCATAAGAGATTTTAT | |
| | CCATGTGGATGATATTTCAAGTGCGCATTTATCGGCTTTGGATTATTTAAAAG | |
| | AGAATGAAAGCAATGTTTTTAATGTAGGTTATGGACATGGTTTTAGCGTAAAA | |
| | GAAGTGATTGAAGCGATGAAAAAAGTTAGCGGAGTGGATTTTAAAGTAGAACT | |
| | TGCCCCACGCCGTGCGGGTGATCCTAGTGTATTGATTTCTGATGCAAGTAAAA | |
| | TCAGAAATCTTACTTCTTGGCAGCCTAAATATGATGATTTAGGGCTTATTTGT | |
| | AAATCTGCTTTTGATTGGGAAAAACAGTGCTTAA | |
| Acinetobacter | ATGGTGAATGAAAATATGAAGCGTTTAGTAGACATAGTCATTTCTTTAATAGC | 7 |
| baumannii | TTTAACTGTTCTGTCGCCAATATTTCTGATAGTTGCTTATAAAGTCCGTAAAA | |
| strain ATCC | ATTTAGGTTCACCAATATTCTTTTACCAAGAAAGACCTGGTAAGGACGGAAAA | |
| 17978 PgIC | TTATTTAAAATGATTAAGTTCCGTTCTATGAAAGATGCATTTGATGCTCAAGG | |
| | AAATCCATTGCCAGATGAAGCTCGTATTACACCATTTGGTCAAAAATTGCGTT | |
| | CAACTAGTCTGGATGAAATGCCGCAGCTCATTAATGTACTAAAAGGTGACATG | |
| | AGCGTAGTGGGTCCGCGTCCAATGTTAAAAGACTTTGTTGCTTTATATTCACC | |
| | CGAACAAGCTCGTCGTTTAGAAGTTCGCCCAGGAATGACGGGTTTAGCTCAGG | |
| | TAAGTGGTCGTAATGAACTTGATTATGAAGAACGATTTAAGTGTGATGTATGG | |
| | TATGTAGATAACCACAACATTTGGGTTGATTTCAAAATCATGTTTAAAACAGT | |
| | CAAAGTGATGTTAAAACGTGAAGGAATCAATGCTCCAGGGCATGTTGGGCCAT | |
| | CTTTATTTAAAGGTAATGATACCCAAGAAAATATTGATTCTTCTGTTAAGTAA | |
| E. coli strain | ATGAAATTTAGCGTTCTGTTATCCCTGTATTACAAAGAGTCTGCACTTTTTCT | 8 |
| O104 | GCATGATTGTTTAGAAAGTATAGCAAGCAATACATGCGCACCTGATCAAATTG | |
| EcWbwC | TAATTGTCTTTGATGGTTATATTAGTGATGATTTGCTACATGTTGTTAATGAA | |
| | TTTTCATTACGTTTACCCATTGATATTGTGCGATTAAGTAATAATGTTGGTCT | |
| | AGGTAAGGCTCTTAATCATGGTCTACAGTATTGTCGCAATGAACTGATTTTTA | |
| | GGATGGATACTGATGATATTTGTTTGCCTGAACGGTTCGCTCTTCAACTTAGC | |
| | TATATGAAATCACACCCTGAAGTTGTTCTTTTGGGAAGTGCCATTGAAGAATT | |
| | TGATAATACAATGAAAATTAGGCAAGGAAAAAGATTTTCAGTTATATACCATG | |
| | AAGATATAAAAAAATTTGCAAAAAAAAGAAATCCTTTCAACCATATGACTGTT | |
| | GTTTTCAGAAAGTCCGTAATTGAAAAGCTTGGTGGATATCAACATCATTATCT | |
| | AATGGAAGATTATAACTTATGGCTTAGGATACTTGCAGCCGATTATCGCACTC | |
| | ATAATTTGAGTGAGGTTCTTGTTAATGTTCGGGCAGGCAGAAATATGCTGTGT | |
| | CGACGGAAAGGTTATTCCTATATTAAGAGTGAGATACTTTTGGCGAAATTAAA | |
| | ATATGAATTACAGTTGGATAATCTGACAGGTGTCATATATACAGGTGTAATTC | |
| | GTATTATCCCTAGAATATTGCCTGTTTCATTACTGAAATTAGTATACAATATA | |
| | TTGCGGAAATAA | |
| Neisseria | ATGCCTGCAGAAACCACCGTTAGCGGTGCACATCCGGCAGCAAAACTGCCGAT | 9 |
| meningitides | TTATATCCTGCCGTGTTTTCTGTGGATTGGTATTGTTCCGTTTACCTTTGCAC | |
| PgIL | TGAAACTGAAACCGAGTCCGGATTTTTATCATGATGCAGCAGCAGCCGCAGGT | |
| (NmPgIL) | CTGATTGTTCTGCTGTTTCTGACCGCAGGTAAAAAACTGTTCGATGTTAAAAT | |
| | TCCGGCAATCAGCTTCCTGCTGTTTGCAATGGCAGCATTTTGGTATCTGCAAG | |
| | CACGTCTGATGAATCTGATTTATCCGGGTATGAATGATATCGTGAGCTGGATC | |
| | TTTATTCTGCTGGCAGTTAGCGCATGGGCATGTCGTAGCCTGGTTGCACATTT | |
| | TGGTCAAGAACGTATTGTTACCCTGTTTGCATGGTCACTGCTGATTGGTAGCC | |
| | TGCTGCAGAGCTGTATTGTTGTTATTCAGTTTGCAGGTTGGGAAGATACACCG | |
| | CTGTTTCAGAACATTATTGTTTATAGCGGTCAGGGTGTGATTGGTCATATTGG | |
| | TCAGCGTAATAATCTGGGCCATTATCTGATGTGGGGTATTCTGGCAGCAGCAT | |
| | ATCTGAATGGTCAGCGCAAAATTCCTGCAGCACTGGGTGTTATTTGTCTGATT | |
| | ATGCAGACCGCAGTTCTGGGTCTGGTTAATAGCCGTACCATTCTGACCTATAT | |
| | TGCAGCAATTGCACTGATTCTGCCGTTTTGGTATTTTCGTAGCGATAAAAGCA | |
| | ATCGTCGTACCATGCTGGGTATTGCCGCAGCAGTTTTTCTGACAGCACTGTTT | |
| | CAGTTCAGCATGAATACAATCCTGGAAACCTTTACCGGTATTCGTTATGAAAC | |
| | CGCAGTTGAACGTGTTGCAAATGGTGGTTTTACCGATCTGCCTCGTCAGATTG | |
| | AATGGAATAAAGCACTGGCAGCCTTTCAGAGCGCACCGATTTTTGGTCATGGT | |
| | TGGAATAGCTTTGCACAGCAGACCTTTCTGATTAATGCCGAACAGCATAACAT | |
| | CTATGATAACCTGCTGAGCAACCTGTTTACCCATAGCCATAATATTGTGCTGC | |
| | AGCTGCTGGCCGAAATGGGTATTAGCGGCACCCTGCTGGTTGCAGCAACCCTG | |
| | CTGACCGGTATTGCGGGTCTGCTGAAACGTCCGCTGACACCGGCAAGCCTGTT | |
| | TCTGATTTGTACCCTGGCCGTTAGCATGTGTCATAGCATGCTGGAATATCCGC | |
| | TGTGGTATGTGTATTTTCTGATTCCGTTTGGTCTGATGCTGTTCCTGAGCCCT | |
| | GCCGAAGCAAGTGATGGTATTGCATTCAAAAAAGCAGCCAATCTGGGTATCCT | |
| | GACCGCAAGCGCAGCAATTTTTGCAGGACTGCTGCACCTGGATTGGACCTATA | |
| | CCCGTCTGGTGAATGCATTTAGTCCGGCAACCGATGATAGCGCAAAAACCCTG | |
| | AATCGTAAAATCAATGAACTGCGCTATATTAGCGCCAATAGCCCGATGCTGAG | |
| | CTTTTATGCAGATTTTAGCCTGGTGAATTTTGCCCTGCCGGAATATCCTGAAA | |
| | CCCAGACCTGGGCAGAAGAAGCCACCCTGAAAAGCCTGAAATATCGTCCGCAT | |
| | AGCGCAACCTATCGTATTGCACTGTATCTGATGCGTCAGGGTAAAGTTGCGGA | |
| | AGCAAAACAGTGGATGCGTGCAACCCAGAGCTATTATCCGTACCTGATGCCTC | |
| | GTTATGCCGATGAAATTCGTAAACTGCCGGTTTGGGCACCGCTGCTGCCTGAA | |
| | CTGCTGAAAGATTGTAAAGCATTTGCAGCCGCACCGGGTCATCCGGAAGCCAA | |
| | ACCGTGTAAAGGTAGCGGTTATCCGTATGATGTTCCGGATTATGCCCATCACC | |
| | ATCACCATCACCATCACCATCACTAA | |
| | (Sequence in bold correspond to HHHHHHHHHH.) | |
| Neissena | ATGAGCGCAGAAACCACCGTTAGCGGTGCACGTCCGGCAGCAAAACTGCCGAT | 10 |
| meningitides | TTATATCCTGCCGTGTTTTCTGTGGATTGGCATTATTCCGTTTACCTTTGCAC | |
| PglO | TOCGTCTGAAACCGAGTCCGGATTTTTATCATGATGCAGCAGCAGCCGCAGGT | |
| (NmPglO) | CTGATTGTTCTGCTGTTTCTGACCGCAGGTAAAAAACTGTTCGATGTTAAAAT | |
| | TCCGGCAATCAGCTTCCTGCTGTTTGCAATGGCAGCATTTTGGTGGCTGCAGG | |
| | CACGTCTGATGAATCTGATTTATCCGGGTATGAATGATATTGCCAGCTGGGTT | |
| | TTTATTCTGCTGGCAGTTAGCGCATGGGCATGTAAAAGCCTGGTTGCACATTA | |
| | TGGTCAAGAACGTATTGTTACCCTGTTTGCATGGTCACTGCTGATTGGTAGCC | |
| | TGCTGCAGAGCTGTATTGTTGTTATTCAGTTTGCAGGCTGGGAAAATACACCG | |
| | CTGCTGCAAAACATTATTGTTCATCGTGGTCAGGGTGTGATTGGTCATATTGG | |
| | TCAGCGTAATAATCTGGGCCATTATCTGATGTGGGGTATTCTGGCAAGCGCAT | |
| | ATCTGAATGGTCAGCGCAAAATTCCTGCAGCACTGGGTGCAATTTGTCTGATT | |
| | ATGCAGACCGCAGTTCTGGGTCTGGTTAATAGCCGTACCATTCTGACCTATAT | |
| | TGCAGCAATTGCACTGATTCTGCCGTTTTGGTATTTTCGTAGCGATAAAAGCA | |
| | ATCGTCGTACCATGCTGGGTATTGCCGCAGCAGTTTTTCTGACAGCACTGTTT | |
| | CAGTTTAGCATGAATGCAATCCTGGAAACCTTTACCGGTATTCGTTATGAAAC | |
| | CGCAGTTGAACGTGTTGCAAATGGTGGTTTTACCGATCTGCCTCGTCAGAGCG | |
| | AATGGAATAAAGCACTGGCAGCCTTTCAGAGCGCACCGATTTTTGGTCATGGT | |
| | TGGAATAGCTTTGCACAGCAGACCTTTCTGATTAATGCCGAACAGCATACCAT | |
| | CCACGATAATTTTCTGAGCACCCTGTTTACCCATAGCCATAACATTATTCTGC | |
| | AGCTGCTGGCCGAAATGGGTATTAGCGGCACCCTGCTGGTTGCAGCAACCCTG | |
| | CTGACCGGTATTGCGGGTCTGCTGAAACGTAGCCTGACACCGGCAAGCCTGTT | |
| | TCTGCTGTGTGCACTGGCCGTTAGCATGTGTCATAGCATGCTGGAATATCCGC | |
| | TGTGGTATGTTTATTTTCTGATTCCGTTTGGCCTGATGCTGTTCCTGAGCCCT | |
| | GCCGAAGCAAGTGATGGTATTGCCTTCAAAAAAGCAGCAAATCTGGGTATCCT | |
| | GACCGCAAGCGCAGCAATTTTTGCAGGACTGCTGCACCTGGATTGGACCTATA | |
| | CCCGTCTGGTGAATAGTTTTTCACCGGCAGCAGATGATAGCGCAAAAACCCTG | |
| | AATCGTAAAATCAATGAACTGCGCTATATTAGCGCCAATAGCCCGATGCTGAG | |
| | CTTTTATGCAGATTTTAGCCTGGTTAATTTTGCCCTGCCGGAATATCCTGAAA | |
| | CCCAGACCTGGGCAGAAGAAGCCACCCTGAAAGCACTGAAATATCGTCCGTAT | |
| | AGCGCAACCTATCGTATTGCACTGTATCTGATGCGTCAGGGTAAAGTTGCGGA | |
| | AGCAAAACAGTGGATGCGTGCAACCCAGAGCTATTATCCGTACCTGATGCCTC | |
| | GTTATGCCGATGAAATTCGTAAACTGCCGGTTTGGGCACCTCTGCTGCCTGAA | |
| | CTGCTGAAAGATTGTAAAGCATTTGCAGCCGCACCGGGTCATCCGGAAACCAA | |
| | ACCGTGTAAATATCCGTATGATGTTCCGGATTATGCGCATCATCACCACCATC | |
| | ACCATCATCATCACTAA | |
| | (Sequence in bold correspond to HHHHHHHHHH.) | |

In some embodiments of the present disclosure, the nucleotide sequences disclosed herein (e.g., the nucleotide sequences encoding the one or more 4-epimerases, the one or more glycosyl-1-phosphate transferases, the one or more O-oligosaccharyltransferases, the one or more ß1,3-galactosyltransferase enzymes, enzymes of an enzymatic pathway capable of producing cytidine-5′-monophosphate-5-N-acetylneuraminic acid (CMP-NeuNAc), and/or the sialyltransferases of the present disclosure) are codon optimized to overcome limitations associated with the codon usage bias between E. coli (and other bacteria) and/or higher organisms, such as yeast and mammalian cells. Codon usage bias refers to differences among organisms in the frequency of occurrence of codons in protein-coding DNA sequences (genes). A codon is a series of three nucleotides (triplets) that encodes a specific amino acid residue in a polypeptide chain. Codon optimization can be achieved by making specific transversion nucleotide changes (i.e., a purine to pyrimidine or pyrimidine to purine nucleotide change) or transition nucleotide change (i.e., a purine to purine or pyrimidine to pyrimidine nucleotide change). Exemplary codon optimized nucleic acid molecules corresponding to C. jejuni Gne (CjGne), Acinetobacter baumannii strain ATCC 17978 PglC, E. coli strain O104 EcWbwC, Neisseria meningitides PglL (NmPglL), and Neisseria meningitides PglO (NmPglO) are set forth herein as SEQ ID NOs: 6-10, SEQ ID NOs: 15-18, SEQ ID NO: 21, and SEQ ID NO: 22.

The nucleotide sequences encoding any of the enzymes disclosed herein (i.e., the nucleotide sequences encoding the one or more 4-epimerases, the one or more glycosyl-1-phosphate transferases, the one or more O-oligosaccharyltransferases, the one or more ß1,3-galactosyltransferase enzymes, enzymes of an enzymatic pathway capable of producing cytidine-5′-monophosphate-5-N-acetylneuraminic acid (CMP-NeuNAc), and/or the sialyltransferases of the present disclosure) may comprise sequences having least 80% identity to any one of SEQ ID NOs: 6-10, SEQ ID NOs: 15-18, SEQ ID NO: 21, and SEQ ID NO: 22.

In another embodiment, the nucleotide sequences of the present disclosure encode a polypeptide having the amino acid sequence of any one or more of SEQ ID NOs: 1-5, or a modified amino acid sequence of any one of SEQ ID NOs: 1-5, where the modified sequence has at least 80% sequence identity to any one of SEQ ID NOs: 1-5.

Methods for transforming/transfecting host cells with recombinant genetic constructs provided herein are well-known in the art and depend on the host system selected, as described in Sambrook et al., Molecular Cloning: A Laboratory Manual, Cold Springs Laboratory Press, Cold Springs Harbor, N.Y. (1989), which is hereby incorporated by reference in its entirety.

As noted above, the further elaboration of Gal-β1,3-GalNAc with additional sugars such as N-Acetylneuraminic acid (NeuNAc) (see FIG. 1A) is also possible. Thus, in some embodiments, when the recombinant prokaryotic host cell does not comprise one or more β1,3-galactosyltransferase enzymes capable of transferring galactose (Gal) to undecaprenyl pyrophosphate (Und-PP)-linked-N-Acetylgalactosamine (GalNac), the recombinant prokaryotic host cell may further expresses: (i) the enzymes of an enzymatic pathway capable of producing cytidine-5′-monophosphate-5-N-acetylneuraminic acid (CMP-NeuNAc); and (ii) one or more α2,6-sialyltransferases (see pathway beginning in the middle of FIG. 1A and continuing to the lower left of FIG. 1A).

In some embodiments, when the recombinant prokaryotic host comprises one or more β1,3-galactosyltransferase enzymes capable of transferring galactose (Gal) to undecaprenyl pyrophosphate (Und-PP)-linked-N-Acetylgalactosamine (GalNac), the recombinant prokaryotic host cell may further expresses: (i) the enzymes of an enzymatic pathway capable of producing cytidine-5′-monophosphate-5-N-acetylneuraminic acid (CMP-NeuNAc); and (ii) one or more α2,3-sialyltransferases and/or one or more α2,6-sialyltransferases (see pathway beginning in the middle of FIG. 1A and continuing to the lower right of FIG. 1A).

Suitable enzymes of an enzymatic pathway capable of producing cytidine-5′-monophosphate-5-N-acetylneuraminic acid (CMP-NeuNAc) are encoded by, e.g., the E. coli neuDBAC genes (e.g., E. coli K1 neuDBAC genes). As described herein, NeuC is a UDP-GlcNAc 2-epimerase that converts UDP-GlcNAc to ManNAc; NeuB is a sialic acid synthase that condenses ManNAc and PEP to form NeuNAc; NeuA is a CMP-NeuNAc synthetase that converts NeuNAc to CMP-NeuNAc; and NeuD promotes efficient sialic acid synthesis by enhancing the activity of NeuC, NeuB, and NeuA (see, e.g., Daines et al., “NeuD Plays a Role in the Synthesis of Sialic Acid in Escherichia coli K1,” FEMS Microbiology Letters 189(2):281-284 (2000) and Valentine et al., “Immunization with Outer Membrane Vesicles Displaying Designer Glycotopes Yields Class-switched, Glycan-specific Antibodies,” Cell. Chem. Biol. 23:655-665 (2016), which are hereby incorporated by reference in their entirety). Thus, in some embodiments, the enzymes of an enzymatic pathway capable of producing CMP-NeuNAc include one or more of NeuA, NeuB, NeuC, and NeuD (e.g., E. coli K1 NeuA, E. coli K1 NeuB, E. coli K1 NeuC, and E. coli K1 NeuD).

The amino acid sequence of E. coli K1 NeuA has the amino acid sequence of SEQ ID NO: 11 below.

(SEQ ID NO: 11)
MRTKIIAIIPARSGSKGLRNKNALMLIDKPLL
AYTIEAALQSEMFEKVIVTTDSEQYGAIAESY
GADFLLRPEELATDKASSFEFIKHALSIYTDY
ENFALLQPTSPFRDSTHLLEAVKLYQTLEKYQ
CVVSVTRSNKPSQIIRPLDDYSTLSFFDLDYS
KYNRNSIVEYHPNGAIFIANKQHYLHTKHFFG
RYSLAYIMDKESSLDIDDRMDFELAITTQQKK
NRQKILYQNIHNRINEKRNBFDSVSDITLIGH
SLFDYWDVKKINDIEVNNLGIAGINSKEYYEY
IIEKERIVNFGEFVFIFFGTNDIVVSDWKKED
TLWYLKKTCQYIKKKNAASKIYLLSVPPVFGR
IDRDNRIINDLNSYLRENVDFAKFISLDHVLK
DSYGNLNKMYTYDGLHFNSNGYTVLENEIAEI
VK

The amino acid sequence of E. coli K1 NeuB has the amino acid sequence of SEQ ID NO: 12 below.

(SEQ ID NO: 12)
MILKAKEAGVNAVKFQTFKADKLISAIAPKAEYQIKNTGELESQLEMTK
KLEMKYDDYLHLMEYAVSLNLDVFSTPFDEDSIDFLASLKQKIWKIPSG
ELLNLPYLEKIAKLPIPDKKIIISTGMATIDEIKQSVSIFINNKVPVDN
ITILHCNTEYPTPFEDVNLNAINDLKKHFPKNNIGFSDHSSGFYAAIAA
VPYGITFIEKHFTLDKSMSGPDHLASIEPDELKHLCIGVRCVEKSLGSN
SKVVTASERKNKIVARKSIIAKTEIKKGEVFSEKNITTKRPGNGISPME
WYNLLGKIAEQDFIPDELIIHSEFKNQGE

The amino acid sequence of E. coli K1 NeuC has the amino acid sequence of SEQ ID NO: 13 below.

(SEQ ID NO: 13)
MKKILYVTGSRAEYGIVRRLLTMLRETPEIQLDLAVTGMHCDNAYGNTIH
IIEQDNFNIIKVVDININTTSHTHILHSMSVCLNSFGDFFSNNTYDAVMV
LGDRYEIFSVAIAASMHNIPLIHIHGGEKTLANYDEFIRHSITKMSKLHL
TSTEEYKKRVIQLGEKPGSVFNIGSLGAENALSLHLPNKQELELKYGSLL
KRYFVVVFHPETLSTQSVNDQIDELLSAISFFKNTHDFIFIGSNADTGSD
IIQRKVKYFCKEYKFRYLISIRSEDYLAMIKCSCGLIGNSSSGLIEVPSL
KVATLNIGDRQKGRVRGASVIDVPVEKNAIVRGINISQDEKFISVVQSSS
NPYFKENALINAVRIIKDFIKSKNKDYKDFYDIPECTTSYD

The amino acid sequence of E. coli K1 NeuD has the amino acid sequence of SEQ ID NO: 14 below.

(SEQ ID NO: 14)
MSKKLIIFGAGGFSKSIIDSLNHKHYELIGFIDKYKSGYHQSYPILGND
IADIENKDNYYYFIGIGKPSTRKHYLNIIRKHNLRLINIIDKTAILSPN
IILGDGIFIGKMCILNRDTRIHDAVVINTRSLIEHGNEIGCCSNISTNV
VLNGDVSVGEETFVGSCTVVNGQLKLGSKSIIGSGSVVIRNIPSNVVVA
GTPTRLIRGNE

Suitable nucleotide sequences encoding the enzymes of an enzymatic pathway capable of producing cytidine-5′-monophosphate-5-N-acetylneuraminic acid (CMP-NeuNAc) disclosed herein are set forth in Table 2 below. Suitable nucleotide sequences also include nucleotide sequences having at least 80%, 81%, 82%, 83%, 84%, 85%, 86%, 87% 88%, 89%, 90%, 91%, 92%, 93%, 94%, 95%, 96%, 97%, 98%, 99%, or 100% sequence identity to the enzymes of an enzymatic pathway capable of producing cytidine-5′-monophosphate-5-N-acetylneuraminic acid (CMP-NeuNAc) coding sequences provided in Table 2 below (i.e., SEQ ID NOs. 15-18).

TABLE 2
Suitable Nucleotide Sequences Encoding the Enzymes of an Enzymatic Pathway
Capable of Producing Cytidine-5′-Monophosphate-5-N-acetylneuraminic acid (CMP-
NeuNAc)

| | | SEQ |
|---|---|---|
| Name | Sequence | ID NO. |
| E. coli K1 | ATGAGAACAAAAATTATTGCGATAATTCCAGCCCGTAGTGGATCTAAAGGGTT | 15 |
| NeuA | GAGAAATAAAAATGCTTTGATGCTGATAGATAAACCTCTTCTTGCTTATACAA | |
| | TTGAAGCTGCCTTGCAGTCAGAAATGTTTGAGAAAGTAATTGTGACAACTGAC | |
| | TCCGAACAGTATGGAGCAATAGCAGAGTCATATGGTGCTGATTTTTTGCTGAG | |
| | ACCGGAAGAACTAGCAACTGATAAAGCATCATCATTTGAATTTATAAAACATG | |
| | CGTTAAGTATATATACTGATTATGAGAACTTTGCTTTATTACAACCAACTTCA | |
| | CCCTTTAGAGATTCGACCCATATTATTGAGGCTGTAAAGTTATATCAAACTTT | |
| | AGAAAAATACCAATGTGTTGTTTCTGTTACTAGAAGCAATAAGCCATCACAAA | |
| | TAATTAGACCATTAGATGATTACTCGACACTGTCTTTTTTTGACCTTGATTAT | |
| | AGTAAATATAATCGAAACTCAATAGTAGAATATCATCCGAATGGAGCTATATT | |
| | TATAGCTAATAAGCAGCATTATCTTCATACAAAGCATTTTTTTGGTCGCTATT | |
| | CACTAGCTTATATTATGGATAAGGAAAGCTCTTTAGATATAGATGATAGAATG | |
| | GATTTCGAACTTGCAATTACCATTCAGCAAAAAAAAAATAGACAAAAAATACT | |
| | TTATCAAAACATACATAATAGAATCAATGAGAAACGAAATGAATTTGATAGTG | |
| | TAAGTGATATAACTTTAATTGGACACTCGCTGTTTGATTATTGGGACGTAAAA | |
| | AAAATAAATGATATAGAAGTTAATAACTTAGGTATCGCTGGTATAAACTCGAA | |
| | GGAGTACTATGAATATATTATTGAGAAAGAGCGGATTGTTAATTTCGGAGAGT | |
| | TTGTTTTCATCTTTTTTGGAACTAATGATATAGTTGTTAGTGATTGGAAAAAA | |
| | GAAGACACATTGTGGTATTTGAAGAAAACATGCCAGTATATAAAGAAGAAAAA | |
| | TGCTGCATCAAAAATTTATTTATTGTCGGTTCCTCCTGTTTTTGGGCGTATTG | |
| | ATCGAGATAATAGAATAATTAATGATTTAAATTCTTATCTTCGAGAGAATGTA | |
| | GATTTTGCGAAGTTTATTAGCTTGGATCACGTTTTAAAAGACTCTTATGGCAA | |
| | TCTAAATAAAATGTATACTTATGATGGCTTACATTTTAATAGTAATGGGTATA | |
| | CAGTATTAGAAAACGAAATAGCGGAGATTGTTAAATGA | |
| E. coli K1 | ATGATATTAAAAGCCAAAGAGGCCGGTGTTAATGCAGTAAAATTCCAAACATT | 16 |
| NeuB | TAAAGCTGATAAATTAATTTCAGCTATTGCACCTAAGGCAGAGTATCAAATAA | |
| | AAAACACAGGAGAATTAGAATCTCAGTTAGAAATGACAAAAAAGCTTGAAATG | |
| | AAGTATGACGATTATCTCCATCTAATGGAATATGCAGTCAGTTTAAATTTAGA | |
| | TGTTTTTTCTACCCCTTTTGACGAAGACTCTATTGATTTTTTAGCATCTTTGA | |
| | AACAAAAAATATGGAAAATCCCTTCAGGTGAGTTATTGAATTTACCGTATCTT | |
| | GAAAAAATAGCCAAGCTTCCGATCCCTGATAAGAAAATAATCATATCAACAGG | |
| | AATGGCTACTATTGATGAGATAAAACAGTCTGTTTCTATTTTTATAAATAATA | |
| | AAGTTCCGGTTGATAATATTACAATATTACATTGCAATACTGAATATCCAACG | |
| | CCCTTTGAGGATGTAAACCTTAATGCTATTAATGATTTGAAAAAACACTTCCC | |
| | TAAGAATAACATAGGCTTCTCTGATCATTCTAGCGGGTTTTATGCAGCTATTG | |
| | CGGCGGTGCCTTATGGAATAACTTTTATTGAAAAACATTTCACTTTAGATAAA | |
| | TCTATGTCTGGCCCAGATCATTTGGCCTCAATAGAACCTGATGAACTGAAACA | |
| | TCTATGTATTGGGGTCAGGTGTGTTGAAAAATCTTTAGGTTCAAATAGTAAAG | |
| | TGGTTACAGCTTCAGAAAGGAAGAATAAAATCGTAGCAAGAAAGTCTATTATA | |
| | GCTAAAACAGAGATAAAAAAAGGTGAGGTTTTTTCAGAAAAAAATATAACAAC | |
| | AAAAAGACCTGGTAATGGTATCAGTCCGATGGAGTGGTATAATTTATTGGGTA | |
| | AAATTGCAGAGCAAGACTTTATTCCAGATGAATTAATAATTCATAGCGAATTC | |
| | AAAAATCAGGGGGAATAA | |
| E. coli K1 | ATGAAAAAAATATTATACGTAACTGGATCTAGAGCTGAATATGGAATAGTTCG | 17 |
| NeuC | GAGACTTTTGACAATGCTAAGAGAAACTCCAGAAATACAGCTTGATTTGGCAG | |
| | TTACAGGAATGCATTGTGATAATGCGTATGGAAATACAATACATATTATAGAA | |
| | CAAGATAATTTTAATATTATCAAGGTTGTGGATATAAATATCAATACAACTTC | |
| | ACATACTCACATTCTCCATTCAATGAGTGTTTGCCTCAATTCGTTTGGTGATT | |
| | TTTTTTCAAATAACACATATGATGCGGTTATGGTTTTAGGCGATAGATATGAA | |
| | ATATTTTCAGTCGCTATCGCAGCATCAATGCATAATATTCCATTAATTCATAT | |
| | TCATGGTGGTGAAAAGACATTAGCTAATTATGATGAGTTTATTAGGCATTCAA | |
| | TTACTAAAATGAGTAAACTCCATCTTACTTCTACAGAAGAGTATAAAAAACGA | |
| | GTAATTCAACTAGGTGAAAAGCCTGGTAGTGTGTTTAATATTGGTTCTCTTGG | |
| | TGCAGAAAATGCTCTTTCATTGCATTTACCAAATAAGCAGGAGTTGGAACTAA | |
| | AATATGGTTCACTGTTAAAACGGTACTTTGTTGTAGTATTCCATCCTGAAACA | |
| | CTTTCCACGCAGTCGGTTAATGATCAAATAGATGAGTTATTGTCAGCGATTTC | |
| | TTTTTTTAAAAATACTCACGACTTTATTTTTATTGGCAGTAACGCTGACACTG | |
| | GTTCTGATATAATTCAGAGAAAAGTAAAATATTTTTGCAAAGAGTATAAGTTC | |
| | AGATATTTGATTTCTATTCGTTCAGAAGATTATTTGGCAATGATTAAATGCTC | |
| | TTGTGGGCTAATTGGGAACTCCTCCTCTGGTTTAATTGAGGTTCCATCTTTAA | |
| | AAGTTGCAACAATTAACATTGGTGATAGGCAGAAAGGCCGTGTTCGTGGAGCC | |
| | AGTGTAATAGATGTACCCGTTGAAAAAAATGCAATCGTCAGAGGGATAAATAT | |
| | ATCTCAAGATGAAAAATTTATTAGTGTTGTACAGTCATCTAGTAATCCTTATT | |
| | TTAAAGAAAATGCTTTAATTAATGCTGTTAGAATTATTAAGGATTTTATTAAA | |
| | TCAAAAAATAAAGATTACAAAGATTTTTATGACATCCCGGAATGTACCACCAG | |
| | TTATGACTAG | |
| E. coli K1 | ATGAGTAAAAAATTAATAATATTTGGTGCGGGTGGTTTTTCAAAATCTATAAT | 18 |
| NeuD | TGACAGCTTAAATCATAAACATTACGAGTTAATAGGATTTATCGATAAATATA | |
| | AAAGTGGTTATCATCAATCATATCCAATATTAGGTAATGATATTGCAGACATC | |
| | GAGAATAAGGATAATTATTATTATTTTATTGGGATAGGGAAACCATCAACTAG | |
| | GAAGCACTATTTAAACATCATAAGAAAACATAATCTACGCTTAATTAACATTA | |
| | TAGATAAAACTGCTATTCTATCACCAAATATTATACTGGGTGATGGAATTTTT | |
| | ATTGGTAAAATGTGTATACTTAACCGTGATACTAGAATACATGATGCCGTTGT | |
| | AATAAATACTAGGAGTTTAATTGAACATGGTAATGAAATAGGCTGCTGTAGCA | |
| | ATATCTCTACTAATGTTGTACTTAATGGTGATGTTTCTGTTGGAGAAGAAACT | |
| | TTTGTTGGTAGCTGTACTGTTGTAAATGGCCAGTTGAAGCTAGGCTCAAAGAG | |
| | TATTATTGGTTCTGGGTCGGTTGTAATTAGAAATATACCAAGTAATGTTGTAG | |
| | TTGCTGGGACTCCAACAAATTAATTAGGGGGAATGAATGA | |

N-acetylneuraminate lyase plays a role in the regulation of sialic acid metabolism in bacterial by catalyzing the reversible aldol cleavage of N-acetylneuraminic acid (sialic acid) to form pyruvate and N-acetyl-D-mannosamine (Izard et al., “The Three-Dimensional Structure of N-Acetylneuraminate Lyase from Escherichia coli,” Structure 2(5):361-369 (1994), which is hereby incorporated by reference in its entirety). Accordingly, N-acetylneuraminate lyase may interfere with the CMP-NeuNAc synthase pathway encoded by the E. coli K1 neuDBAC genes. Thus, in some embodiments, the recombinant prokaryotic host cell does not express an enzymatically active N-acetylneuraminate lyase. Exemplary N-acetylneuraminate lyases are well known in the art and include, without limitation, NanA.

In some embodiments, the recombinant prokaryotic host cell is an E. coli host cell that lacks a functional copy of the nanA gene (Valentine et al., “Immunization with Outer Membrane Vesicles Displaying Designer Glycotopes Yields Class-switched, Glycan-specific Antibodies,” Cell. Chem. Biol. 23:655-665 (2016), which is hereby incorporated by reference in its entirety).

An exemplary sialyltransferases for use in the present disclosure are identified in the schematic of FIG. 1A and include, e.g., an α2,6-sialyltransferases and an α2,3-sialyltransferase. Suitable α2,6-sialyltransferases include the α2,6-sialyltransferase from Photobacterium sp. JT-ISH-224.

The amino acid sequence of Photobacterium sp. JT-ISH-224 α2,6-sialyltransferases (PspST6) has the amino acid sequence of SEQ ID NO: 19 below.

(SEQ ID NO: 19)
MSEENTQSIIKNDINKTIIDEEYVNLEPINQSNISFTKHSWVQTCGTQQL
LTEQNKESISLSVVAPRLDDDEKYCFDFNGVSNKGEKYITKVTLNVVAPS
LEVYVDHASLPTLQQLMDIIKSEEENPTAQRYIAWGRIVPTDEQMKELNI
TSFALINNHTPADLVQEIVKQAQTKHRLNVKLSSNTAHSFDNLVPILKEL
NSFNNVTVTNIDLYDDGSAEYVNLYNWRDTLNKTDNLKIGKDYLEDVING
INEDTSNTGTSSVYNWQKLYPANYHFLRKDYLTLEPSLHELRDYIGDSLK
QMQWDGFKKFNSKQQELFLSIVNFDKQKLQNEYNSSNLPNFVFTGTTVWA
GNHEREYYAKQQINVINNAINESSPHYLGNSYDLFFKGHPGGGIINTLIM
QNYPSMVDIPSKISFEVLMMTDMLPDAVAGIASSLYFTIPAEKIKFIVFT
STETITDRETALRSPLVQVMIKLGIVKEENVLFWADLPNCETGVCIAV

In some embodiments, the one or more sialyltransferases is an α2,3-sialyltransferase. In accordance with such embodiments, the one or more α2,3-sialyltransferases is WbwA from E. coli O104.

The amino acid sequence of E. coli O104 α2,3-sialyltransferases (EcWbwA) has the amino acid sequence of SEQ ID NO: 20 below.

(SEQ ID NO: 20)
MKMRNNVFVFDSPYCLLIYCILFNHQLHDTIY
IYSDNVSLRDINFPGKERYIIKKGNNKTSKIF
YYFCFLFRLIINPKLRKLISNRKDYRFLGQDH
LFFSKPFLNEFILLEDGLANYRYPHYSRLYKL
IIGGHTFGRSTKVNKILLSGMIDIIDPTINDK
VEFFDLIAAWEKLNSVQKKEINHIFNYCIEDE
LVADVMILTQPLSEDGFISENEKIRLYDEIIK
EYDDKKIVIRQHPRELTDYSLYFKDVKVNKVK
APVELVVLNSPMIKTAVTLFSGGIFNIPCREK
IFKGTSFSPLLSKHFGNISNKFEVKGKK

Suitable nucleotide sequences encoding the sialyltransferases disclosed herein are set forth in Table 3 below. Suitable nucleotide sequences also include nucleotide sequences having at least 80%, 81%, 82%, 83%, 84%, 85%, 86%, 87%, 88%, 89%, 90%, 91%, 92%, 93%, 94%, 95%, 96%, 97%, 98%, 99%, or 100% sequence identity to the sialyltransferase coding sequences provided in Table 3 below (i.e., SEQ ID NOs. 21-22).

TABLE 3
Suitable Sialyltransferase Coding Sequences

| | | SEQ |
|---|---|---|
| Name | Sequence | ID NO. |
| Photobacterium sp. | ATGAGCGAAGAAAATACCCAGAGCATCATCAAAAACGATATCAACAAA | 21 |
| JT-ISH-224 α2,6- | ACCATCATCGACGAAGAGTATGTTAACCTGGAACCGATTAATCAGAGC | |
| sialyltransferases | AACATCAGCTTTACCAAACATAGCTGGGTTCAGACCTGTGGCACCCAG | |
| (PspST6) | CAACTGCTGACAGAACAGAATAAAGAAAGCATTAGCCTGAGCGTTGTT | |
| | GCACCGCGTCTGGATGATGATGAGAAATATTGCTTTGATTTCAACGGC | |
| | GTGAGCAACAAAGGCGAAAAATACATTACCAAAGTGACCCTGAATGTT | |
| | GTGGCACCGAGCCTGGAAGTTTATGTTGATCATGCAAGCCTGCCGACC | |
| | CTGCAGCAGCTGATGGATATTATCAAAAGCGAAGAAGAAAATCCGACC | |
| | GCACAGCGTTATATTGCATGGGGTCGTATTGTTCCGACCGATGAGCAG | |
| | ATGAAAGAACTGAATATTACCAGCTTTGCCCTGATCAATAATCATACA | |
| | CCGGCAGATCTGGTTCAAGAAATTGTTAAACAGGCACAGACCAAACAT | |
| | CGTCTGAATGTTAAACTGAGCAGCAATACCGCACATAGCTTTGATAAT | |
| | CTGGTTCCGATTCTGAAAGAGCTGAACAGCTTTAATAACGTGACCGTG | |
| | ACCAATATCGATCTGTACGATGATGGCAGCGCAGAATATGTGAATCTG | |
| | TATAATTGGCGTGACACCCTGAATAAAACCGACAATCTGAAAATCGGC | |
| | AAAGATTACCTGGAAGATGTGATTAACGGCATCAATGAAGATACCAGT | |
| | AATACCGGCACCAGCAGCGTTTATAACTGGCAGAAACTGTATCCGGCA | |
| | AACTATCATTTTCTGCGCAAAGACTATCTGACACTGGAACCGTCCCTG | |
| | CATGAACTGCGTGATTATATTGGTGATAGCCTGAAACAAATGCAGTGG | |
| | GATGGTTTCAAAAAATTCAACAGCAAACAGCAAGAACTGTTCCTGAGC | |
| | ATTGTGAACTTCGATAAACAGAAACTGCAGAACGAATACAATAGCAGC | |
| | AATCTGCCGAATTTTGTGTTTACCGGTACAACCGTTTGGGCAGGTAAT | |
| | CATGAACGCGAATATTATGCAAAACAGCAGATCAACGTGATCAACAAC | |
| | GCAATTAATGAAAGCAGTCCGCATTATCTGGGTAATAGCTATGACCTG | |
| | TTTTTCAAAGGTCATCCGGGTGGTGGTATTATCAATACCCTGATTATG | |
| | CAGAATTATCCGAGCATGGTTGATATCCCGAGCAAAATTAGCTTTGAA | |
| | GTTCTGATGATGACCGATATGCTGCCGGATGCCGTTGCAGGTATTGCA | |
| | AGCAGCUTGTATTTTACCATTCCGGCAGAAAAAATCAAATTCATCGTT | |
| | TTCACCAGCACCGAAACCATTACCGATCGTGAAACCGCACTGCGTAGT | |
| | CCGCTGGTTCAGGTTATGATTAAACTGGGTATTGTGAAAGAGGAAAAC | |
| | GTCCTGTTTTGGGCAGACCTGCCGAACTGTGAAACCGGTGTTTGTATT | |
| | GCAGTTTAA | |
| E. coli O104 α2,3- | ATGAAGATGCGTAACAACGTATTCGTCTTCGACTCACCGTACTGCCTT | 22 |
| sialyltransferases | TTAATTTATTGCATCCTTTTCAACCATCAGCTTCACGATACAATCTAC | |
| (EcWbwA) | ATTTACTCGGACAACGTCTCATTGCGTGATATTAACTTTCCTGGGAAA | |
| | GAGCGCTATATCATCAAAAAAGGGAACAACAAAACAAGTAAAATCTTC | |
| | TATTACTTCTGCTTCTTGTTCCGTCTTATCATTAACCCTAAGCTGCGT | |
| | AAATTAATCTCTAATCGTAAAGACTACCGCTTCCTTGGCCAGGACCAC | |
| | TTGTTCTTTTCAAAGCCTTTCTTAAATGAGTTCATCCTGCTTGAGGAT | |
| | GGTTTAGCCAACTATCGTTATCCTCACTACTCTCGCCTTTACAAGTTA | |
| | ATCATTGGGGGGCACACGTTTGGCCGTAGTACCAAGGTAAACAAGATC | |
| | TTGTTAAGTGGAATGATCGACATCATTGACCCCACTATCAATGACAAG | |
| | GTGGAGTTTTTCGACTTGATCGCTGCGTGGGAGAAGCTGAACAGTGTG | |
| | CAGAAGAAGGAAATTAATCATATCTTCAACTATTGTATCGAGGATGAA | |
| | CTTGTTGCTGATGTGATGATTCTGACTCAACCATTGAGCGAAGACGGC | |
| | TTTATCTCGGAGAATGAAAAAATTCGCTTGTATGACGAAATCATTAAA | |
| | GAATACGACGATAAAAAAATTGTTATCCGCCAACACCCGCGTGAGTTG | |
| | ACCGATTACTCCTTGTATTTTAAGGACGTCAAGGTTAATAAAGTAAAG | |
| | GCCCCTGTTGAATTAGTCGTACTTAATAGCCCGATGATTAAGACCGCT | |
| | GTAACTTTATTCAGCGGTGGAATCTTCAACATTCCATGTCGTGAGAAA | |
| | ATCTTTAAGGGAACCTCGTTTTCTCCTCTTCTGAGTAAACACTTCGGT | |
| | AATA1-AGTAALAAGTTCAGGTAAAGGGAAAGAAATAA | |

As noted above, in some embodiments, the enzymes of the enzymatic pathway capable of producing cytidine-5′-monophosphate-5-N-acetylneuraminic acid (CMP-NeuNAc) are encoded by a poly-nucleotide sequence that is located on an extrachromosomal plasmid carried by the prokaryotic host cell. As described herein, expression of the enzymes of the enzymatic pathway capable of producing cytidine-5′-monophosphate-5-N-acetylneuraminic acid (CMP-NeuNAc) from an extrachromosomal plasmid is effective to produce greater amounts of the enzymes of the enzymatic pathway capable of producing cytidine-5′-monophosphate-5-N-acetylneuraminic acid (CMP-NeuNAc) than when the enzymes of the enzymatic pathway capable of producing cytidine-5′-monophosphate-5-N-acetylneuraminic acid (CMP-NeuNAc) are expressed in the recombinant prokaryotic host cell's genome.

In some embodiments, the enzymes of an enzymatic pathway capable of producing cytidine-5′-monophosphate-5-N-acetylneuraminic acid (CMP-NeuNAc) are encoded by a polynucleotide sequence that is located in the recombinant prokaryotic host cell's genome. Thus, in some embodiments, the prokaryotic host cell is an E. coli cell and the one or more enzymes of an enzymatic pathway capable of producing cytidine-5′-monophosphate-5-N-acetylneuraminic acid (CMP-NeuNAc) (e.g., E. coli K1 NeuA, E. coli K1 NeuB, E. coli K1 NeuC, and E. coli K1 NeuD) are encoded by a polynucleotide sequence that is located at the native O-antigen locus of the host cell's genome. In accordance with such embodiments, the gnomically integrated enzymatic pathway capable of producing cytidine-5′-monophosphate-5-N-acetylneuraminic acid (CMP-NeuNAc) is heterologous and orthogonal to the host cell. As described herein, the gnomically integrated enzymatic pathway capable of producing cytidine-5′-monophosphate-5-N-acetylneuraminic acid (CMP-NeuNAc), when integrated into the native O-antigen locus of the host cell genome, produces greater amounts of glycoprotein bearing sialic acid than when (i) the enzymatic pathway capable of producing cytidine-5′-monophosphate-5-N-acetylneuraminic acid (CMP-NeuNAc) is integrated at any other locus in the host cell genome and/or (ii) the enzymatic pathway capable of producing cytidine-5′-monophosphate-5-N-acetylneuraminic acid (CMP-NeuNAc) is located on an extrachromosomal plasmid carried by the host cell.

In some embodiments, each of the one or more α2,3-sialyltransferases or α2,6-sialyltransferases is encoded by a polynucleotide sequence that is independently located either on an extrachromosomal plasmid carried by the prokaryotic host cell or in the recombinant prokaryotic host cell's genome.

In some embodiments of present disclosure, the recombinant prokaryotic host cell further expresses a “glycoprotein target” or a “target protein for glycosylation.” As used herein, the terms “glycoprotein target” or “glycoprotein target” refer to a protein of interest which comprises one or more acceptor sites for O-glycosylation. Thus, in some embodiments, the glycoprotein target comprises one or more serine and/or threonine residue. Suitable target proteins include prokaryotic and eukaryotic proteins. In some embodiments, the glycoprotein target is a mucin or mucin-like protein. Exemplary mucins and mucin-like proteins are well known in the art and include, e.g., MUC1, MUC2, MUC3, MUC4, MUC5, MUC6, MUC7, MUC8, MUC9, MUC10, MUC11, MUC12, MUC13, MUC14, MUC15, MUC16, MUC17, MUC18, MUC19, MUC20, MUC21, MUC22, MUC23, MUC24, MUC25, MUC26, MUC27, MUC28, MUC29, MUC30, MUC31, MUC32, MUC33, MUC 34, MUC35, MUC26, MUC37, MUC38, MUC39, MUC 40, MUC41, MUC42, human erythropoietin (EPO), human glycophorin C (GPC), and fragments thereof, as well as leukosialin (leucocyte sialoglycoprotein, sialophorin, CD43, galactoglycoprotein, GALGP)(Schmid et al., “Amino Acid Sequence of Human Plasma Galactoglycoprotein: Identity with the Extracellular Region of CD43 (Sialophorin),” Proc. Natl. Adad. Sci. USA 89(2):663-667 (1992); Fukada, M., “Leukosialin, A Major O-Glycan-Containing Sialoglycoprotein Defining Leukocyte Differentiation and Malignancy,” Glycobiology 1(4):347-356 (1991); and Campos et al., “Probing the O-Glycosylation of Gastric Cancer Cell Lines for Biomarker Discovery;” Mol. Cell. Proteomics 14(6):P1616-1629 (2015), which are hereby incorporated by reference in their entirety); glycophorin A (PAS-2, sialoglycoprotein alpha, MN sialoglycoprotein) (Pisano et al., “Glycosylation Sites Identified by Solid-Phase Edman Degradation: O-Linked Glycosylation Motifs on Human Glycophorin A,” Glycobiology 3(5):429-435 (1993), which is hereby incorporated by reference in its entirety); Glycophorin C (Glycophorin D, PAS-2′, GLPC) (Dahr & Beyreuther, “A Revision of the N-Terminal Structure of Sialoglycoprotein D (Glycophorin C) from Human Erythrocyte Membranes,” Biol. Chem. Hoppe Seyler 366(11):1067-1070 (1985), which is hereby incorporated by reference in its entirety); Von willebrand factor (Titani et al., “Amino Acid Sequence of Human von Willebrand Factor,” Biochemistry 25(11):3171-3184 (1986) and Samor et al., “Primary Structure of the Major O-Glycosidically Linked Carbohydrate Unit of Human von Willebrand Factor,” Glyconj. J. 6(3):263-270 (1989), which is hereby incorporated by reference in its entirety); Kininogen (Lottspeich et al., “The Amino Acid Sequence of the Light Chain of Human High-Molecular-Mass Kininogen,” Eur. J. Biochem. 152(2):307-314 (1985), which is hereby incorporated by reference in its entirety); and chorionic gonadotropin beta chain (Birken et al., “Characterization of Antisera Distinguishing Carbohydrate Structures in the Beta-Carboxyl-Terminal Region of Human Chorionic Gonadotropin,” Endocrinology 122(5):2054-2063 (1988), which is hereby incorporated by reference in its entirety) (see Table 4 below).

TABLE 4
Suitable Glycoprotein Target Sequences

| | | SEQ |
|---|---|---|
| Name | Sequence | ID NO. |
| MUC1 (mucin 1, | MTPGTQSPFFLLLLLTVLTVVTGSGHASSTPGGEKETSATQRSSVPSS | 48 |
| polymorphic | TEKNAVSMTSSVLSSHSPGSGSSTTQGQDVTLAPATEPASGSAATWGQ | |
| epithelial mucin, | DVTSVPVTRPALGSTTPPAHDVTSAPDNKPAPGSTAPPAHGVTSAPDT | |
| PEM, PEMT, | RPAPGSTAPPAHGVTSAPDTRPAPGSTAPPAHGVTSAPDTRPAPGSTA | |
| episialin tumor- | PPAHGVTSAPDTRPAPGSTAPPAHGVTSAPDTRPAPGSTAPPAHGVTS | |
| associated mucin, | APDTRPAPGSTAPPAHGVTSAPDTRPAPGSTAPPAHGVTSAPDTRPAP | |
| carcinoma- | GSTAPPAHGVTSAPDTRPAPGSTAPPAHGVTSAPDTRPAPGSTAPPAH | |
| associated mucin, | GVTSAPDTRPAPGSTAPPAHGVTSAPDTRPAPGSTAPPAHGVTSAPDT | |
| tumor-associated | RPAPGSTAPPAHGVTSAPDTRPAPGSTAPPAHGVTSAPDTRPAPGSTA | |
| epithelial membrane | PPAHGVTSAPDTRPAPGSTAPPAHGVTSAPDTRPAPGSTAPPAHGVTS | |
| antigen, EMA, | APDTRPAPGSTAPPAHGVTSAPDTRPAPGSTAPPAHGVTSAPDTRPAP | |
| H23ag, peanut- | GSTAPPAHGVTSAPDTRPAPGSTAPPAHGVTSAPDTRPAPGSTAPPAH | |
| reactive urinary | GVTSAPDTRPAPGSTAPPAHGVTSAPDTRPAPGSTAPPAHGVTSAPDT | |
| mucin, PUM) | RPAPGSTAPPAHGVTSAPDTRPAPGSTAPPAHGVTSAPDTRPAPGSTA | |
| | PPAHGVTSAPDTRPAPGSTAPPAHGVTSAPDTRPAPGSTAPPAHGVTS | |
| | APDTRPAPGSTAPPAHGVTSAPDTRPAPGSTAPPAHGVTSAPDTRPAP | |
| | GSTAPPAHGVTSAPDTRPAPGSTAPPAHGVTSAPDTRPAPGSTAPPAH | |
| | GVTSAPDTRPAPGSTAPPAHGVTSAPDTRPAPGSTAPPAHGVTSAPDT | |
| | RPAPGSTAPPAHGVTSAPDTRPAPGSTAPPAHGVTSAPDTRPAPGSTA | |
| | PPAHGVTSAPDTRPAPGSTAPPAHGVTSAPDNRPALGSTAPPVHNVTS | |
| | ASGSASGSASTLVHNGTSARATTTPASKSTPFSIPSHHSDTPTTLASH | |
| | STKTDASSTHHSSVPPLTSSNHSTSPQLSTGVSFFFLSFHISNLQFNS | |
| | SLEDPSTDYYQELQRDISEMFLQIYKQGGFLGLSNIKFRPGSVVVQLT | |
| | LAFREGTTNVHDVETQPNQYKTEAASRYNLTISDVSVSDVPFPFSAQS | |
| | GAGVPGWGIALLVLVCVLVALAIVYLIALAVCQCRRKNYGQLDIFPAR | |
| | DTYHPMSEYPTYHTHGRYVPPSSTDRSPYEKVSAGNGGSSLSYTNPAV | |
| | AAASANL | |
| Leukosialin | MATLLLLLGVLVVSPDALGSTTAVQTPTSGEPLVSTSEPLSSKMYTTS | 49 |
| (leucocyte | ITSDPKADSTGDQTSALPPSTSINEGSPLWTSIGASTGSPLPEPTTYQ | |
| sialoglycoprotein, | EVSIKMSSVPQETPHATSHPAVPITANSLGSHTVTGGTITTNSPETSS | |
| sialophorin, CD43, | RTSGAPVTTAASSLETSRGTSGPPLTMATVSLETSKGTSGPPVTMATD | |
| galactoglycoprotein, | SLETSTGTTGPPVTMTTGSLEPSSGASGPQVSSVKLSTMMSPTTSTNA | |
| GALGP) | STVPFRNPDENSRGMLPVAVLVALLAVIVLVALLLLWRRRQKRRTGAL | |
| | VLSRGGKRNGVVDAWAGPAQVPEEGAVTVTVGGSGGDKGSGFPDGEGS | |
| | SRRPTLTTFFGRRKSRQGSLAMEELKSGSGPSLKGEEEPLVASEDGAV | |
| | DAPAPDEPEGGDGAAP | |
| Glycophorin A | MYGKIIFVLLLSAIVSISASSTTGVAMHTSTSSSVTKSYISSQTNDTH | 50 |
| (PAS-2, | KRDTYAATPRAHEVSEISVRTVYPPEEETGERVQLAHHFSEPEITLII | |
| sialoglycoprotein | FGVMAGVIGTILLISYGIRRLIKKSPSDVKPLPSPDTDVPLSSVEIEN | |
| alpha, MN | PETSDQ | |
| sialogl ycoprotein) | | |
| Glycophorin C | MWSTRSPNSTAWPLSLEPDPGMASASTTMHTTTIAEPDPGMSGWPDGR | 51 |
| (Glycophorin D, | METSTPTIMDIVVIAGVIAAVAIVLVSLLFVMLRYMYRHKGTYHTNEA | |
| PAS-2′, GLPC) | KGTEFAESADAALQGDPALQDAGDSSRKEYFI | |
| Von willebrand | MIPARFAGVLLALALILPGTLCAEGTRGRSSTARCSLFGSDFVNTFDG | 52 |
| factor | SMYSFAGYCSYLLAGGCQKRSFSIIGDFQNGKRVSLSVYLGEFFDIHL | |
| | FVNGTVTQGDQRVSMPYASKGLYLETEAGYYKLSGEAYGFVARIDGSG | |
| | NFQVLLSDRYFNKTCGLCGNFNIFAEDDFMTQEGTLTSDPYDFANSWA | |
| | LSSGEQWCERASPPSSSCNISSGEMQKGLWEQCQLLKSTSVFARCHPL | |
| | VDPEPFVALCEKTLCECAGGLECACPALLEYARTCAQEGMVLYGWTDH | |
| | SACSPVCPAGMEYRQCVSPCARTCQSLHINEMCQERCVDGCSCPEGQL | |
| | LDEGLCVESTECPCVHSGKRYPPGTSLSRDCNTCICRNSQWICSNEEC | |
| | PGECLVTGQSHFKSFDNRYFTFSGICQYLLARDCQDHSFSIVIETVQC | |
| | ADDRDAVCTRSVTVRLPGLHNSLVKLKHGAGVAMDGQDIQLPLLKGDL | |
| | RIQHTVTASVRLSYGEDLQMDWDGRGRLLVKLSPVYAGKTCGLCGNYN | |
| | GNQGDDFLTPSGLAEPRVEDFGNAWKLHGDCQDLQKQHSDPCALNPRM | |
| | TRFSEEACAVLTSPTFEACHRAVSPLPYLRNCRYDVCSCSDGRECLCG | |
| | ALASYAAACAGRGVRVAWREPGRCELNCPKGQVYLQCGTPCNLTCRSL | |
| | SYPDEECNEACLEGCFCPPGLYMDERGDCVPKAQCPCYYDGEIFQPED | |
| | IFSDHHTMCYCEDGFMHCTMSGVPGSLLPDAVLSSPLSHRSKRSLSCR | |
| | PPMVKLVCPADNLRAEGLECTKTCQNYDLECMSMGCVSGCLCPPGMVR | |
| | HENRCVALERCPCFHQGKEYAPGETVKIGCNTCVCRDRKWNCTDHVCD | |
| | ATCSTIGMAHYLTFDGLKYLFPGECQYVLVQDYCGSNPGTFRILVGNK | |
| | GCSHPSVKCKKRVTILVEGGEIELFDGEVNVKRPMKDETHFEVVESGR | |
| | YIILLLGKALSVVWDRHLSISVVLKQTYQEKVCGLCGNFDGIQNNDLT | |
| | SSNLQVEEDPVDFGNSWKVSSQCADTRKVPLDSSPATCHNNIMKQTMV | |
| | DSSCRILTSDVFQDCNKLVDPEPYLDVCIYDTCSCESIGDCACFCDTI | |
| | AAYAHVCAQHGKVVTWRTATLCPQSCEERNLRENGYECEWRYNSCAPA | |
| | CQVTCQHPEPLACPVQCVEGCHAHCPPGKILDELLQTCVDPEDCPVCE | |
| | VAGRRFASGKKVTLNPSDPEHCQICHCDVVNLTCEACQEPGGLVVPPT | |
| | DAPVSPTTLYVEDISEPPLHDFYCSRLLDLVFLLDGSSRLSEAEFEVL | |
| | KAFVVDMMERLRISQKWVRVAVVEYHDGSHAYIGLKDRKRPSELRRIA | |
| | SQVKYAGSQVASTSEVLKYTLFQIFSKIDRPEASRIALLLMASQEPQR | |
| | MSRNFVRYVQGLKKKKVIVIPVGIGPHANLKQIRLIEKQAPENKAFVL | |
| | SSVDELEQQRDEIVSYLCDLAPEAPPPTLPPHMAQVTVGPGLLGVSTL | |
| | GPKRNSMVLDVAFVLEGSDKIGEADFNRSKEFMEEVIQRMDVGQDSIH | |
| | VTVLQYSYMVTVEYPFSEAQSKGDILQRVREIRYQGGNRTNTGLALRY | |
| | LSDHSFLVSQGDREQAPNLVYMVTGNPASDEIKRLPGDIQVVPIGVGP | |
| | NANVQELERIGWPNAPILIQDFETLPREAPDLVLQRCCSGEGLQIPTL | |
| | SPAPDCSQPLDVILLLDGSSSFPASYFDEMKSFAKAFISKANIGPRLT | |
| | QVSVLQYGSITTIDVPWNVVPEKAHLLSLVDVMQREGGPSQIGDALGF | |
| | AVRYLTSEMHGARPGASKAVVILVTDVSVDSVDAAADAARSNRVTVEP | |
| | IGIGDRYDAAQLRILAGPAGDSNVVKLQRIEDLPTMVTLGNSFLHKLC | |
| | SGFVRICMDEDGNEKRPGDVWTLPDQCHTVTCQPDGQTLLKSHRVNCD | |
| | RGLRPSCPNSQSPVKVEETCGCRWTCPCVCTGSSTRHIVTFDGQNFKL | |
| | TGSCSYVLFQNKEQDLEVILHNGACSPGARQGCMKSIEVKHSALSVEL | |
| | HSDMEVTVNGRLVSVPYVGGNMEVNVYGAIMHEVRFNHLGHIFTFTPQ | |
| | NNEFQLQLSPKTFASKTYGLCGICDENGANDFMLRDGTVTTDWKTLVQ | |
| | EWTVQRPGQTCQPILEEQCLVPDSSHCQVLLLPLFAECHKVLAPATFY | |
| | AICQQDSCHQEQVCEVIASYAHLCRTNGVCVDWRTPDFCAMSCPPSLV | |
| | YNHCEHGCPRHCDGNVSSCGDHPSEGCFCPPDKVMLEGSCVPEEACTQ | |
| | CIGEDGVQHQFLEAWVPDHQPCQICTCLSGRKVNCTTQPCPTAKAPTC | |
| | GLCEVARLRQNADQCCPEYECVCDPVSCDLPPVPHCERGLQPTLTNPG | |
| | ECRPNFTCACRKEECKRVSPPSCPPHRLPTLRKTQCCDEYECACNCVN | |
| | STVSCPLGYLASTATNDCGCTTTTCLPDKVCVHRSTIYPVGQFWEEGC | |
| | DVCTCTDMEDAVMGLRVAQCSQKPCEDSCRSGFTYVLHEGECCGRCLP | |
| | SACEVVTGSPRGDSQSSWKSVGSQWASPENPCLINECVRVKEEVFIQQ | |
| | RNVSCPQLEVPVCPSGFQLSCKTSACCPSCRCERMEACMLNGTVIGPG | |
| | KTVMIDVCTTCRCMVQVGVISGFKLECRKTTCNPCPLGYKEENNTGEC | |
| | CGRCLPTACTIQLRGGQIMTLKRDETLQDGCDTHFCKVNERGEYFWEK | |
| | RVTGCPPFDEHKCLAEGGKIMKIPGTCCDTCEEPECNDITARLQYVKV | |
| | GSCKSEVEVDIHYCQGKCASKAMYSIDINDVQDQCSCCSPTRTEPMQV | |
| | ALHCTNGSVVYHEVLNAMECKCSPRKCSK | |
| Kininogen | MKLITILFLCSRLLLSLTQESQSEEIDCNDKDLFKAVDAALKKYNSQN | 53 |
| | QSNNQFVLYRITEATKTVGSDTFYSFKYEIKEGDCPVQSGKTWQDCEY | |
| | KDAAKAATGECTATVGKRSSTKFSVATQTCQITPAEGPVVTAQYDCLG | |
| | CVHPISTQSPDLEPILRHGIQYFNNNTQHSSLFMLNEVKRAQRQVVAG | |
| | LNFRITYSIVQTNCSKENFLFLTPDCKSLWNGDTGECTDNAYIDIQLR | |
| | IASFSQNCDIYPGKDFVQPPTKICVGCPRDIPTNSPELEETLTHTITK | |
| | LNAENNATFYFKIDNVKKARVQVVAGKKYFIDFVARETTCSKESNEEL | |
| | TESCETKKLGQSLDCNAEVYVVPWEKKIYPTVNCQPLGMISLMKRPPG | |
| | FSPFRSSRIGEIKEETTVSPPHTSMAPAQDEERDSGKEQGHTRRHDWG | |
| | HEKQRKHNLGHGHKHERDQGHGHQRGHGLGHGHEQQHGLGHGHKPKLD | |
| | DDLEHQGGHVLDHGHKHKHGHGHGKHKNKGKKNGKHNGWKTEHLASSS | |
| | EDSTTPSAQTQEKTEGPTPIPSLAKPGVTVTFSDFQDSDLIATMMPPI | |
| | SPAPIQSDDDWIPDIQTDPNGLSFNPISDFPDTTSPKCPGRPWKSVSE | |
| | INPTTQMKESYYFDLTDGLS | |
| Chorionic | MEMFQGLLLLLLLSMGGTWASKEPLRPRCRPINATLAVEKEGCPVCIT | 54 |
| gonadotropin beta | VNTTICAGYCPTMTRVLQGVLPALPQVVCNYRDVRFESIRLPGCPRGV | |
| chain | NPVVSYAVALSCQCALCRRSTTDCGGPKDHPLTCDDPRFQDSSSSKAP | |
| | PPSLPSPSRLPGPSDTPILPQ | |

Aberrant mucin expression and glycosylation are reliable biomarkers of carcinomas in humans (Rata et al., “MUC Glycoproteins: Potential Biomarkers and Molecular Targets for Cancer Therapy,” Curr. Cancer Drug Targets 21(2):132-152 (2021), which is hereby incorporated by reference in its entirety). Indeed, the membrane-associated mucin MUC1 is aberrantly expressed in −60% of all cancers diagnosed each year in the U.S. (Jonckheere & Van Seuningen, “The Membrane-Bound Mucins: From Cell Signalling to Transcriptional Regulation and Expression in Epithelial Cancers,” Biochimie 92(1):1-11 (2009), which is hereby incorporated by reference in its entirety), rendering MUC1 one of the most prominently dysregulated genes in cancer. Another mucin, MUC16 (also called CA125), is highly expressed in ovarian cancer and clinically used as a biomarker for treatment efficacy and surveillance.

Additional suitable glycoprotein targets may be selected from the group consisting of a therapeutic protein, a diagnostic protein, an industrial enzyme, or a portion thereof.

In some embodiments, the therapeutic protein is selected from the group consisting of an enzyme, a cytokine, a hormone, a growth factor, an inhibitor protein, a protein receptor, a ligand that binds a protein receptor, or an antibody.

In some embodiments, the target protein is heterologous to the recombinant prokaryotic host cell.

In some embodiments, the glycoprotein target comprises a MOOR tag (WPAAASAP (SEQ ID NO: 24).

In some embodiments, the target protein is encoded by a polynucleotide sequence that is located on an extrachromosomal plasmid carried by the prokaryotic host cell or in the recombinant prokaryotic host cell's genome.

In some embodiments, the expression of one or more of: the one or more 4-epimerases, the one or more glycosyl-1-phosphate transferases, the one or more O-oligosaccharyltransferases, the one or more ß1,3-galactosyltransferase enzymes capable of transferring galactose (Gal) to undecaprenyl pyrophosphate (Und-PP)-linked N-Acetylgalactosamine (GalNAc), the glycoprotein target comprising one or more serine and/or threonine residues, the one or more α2,3-sialyltransferases, and/or the one or more α2,6-sialyltransferases is constitutive.

In some embodiments, the expression of one or more of: the one or more 4-epimerases, the one or more glycosyl-1-phosphate transferases, the one or more O-oligosaccharyltransferases, the one or more ß1,3-galactosyltransferase enzymes capable of transferring galactose (Gal) to undecaprenyl pyrophosphate (Und-PP)-linked N-Acetylgalactosamine (GalNAc), the glycoprotein target comprising one or more serine and/or threonine residues, the one or more α2,3-sialyltransferases, and the one or more α2,6-sialyltransferases is inducible.

In some embodiments, membrane extracts may be prepared from the recombinant prokaryotic host cells disclosed herein. The membrane extracts may be prepared from different recombinant prokaryotic host cell strains as disclosed herein and the membrane extracts may be combined to prepare a mixed membrane extract. In some embodiments, one or more membrane extracts may be prepared from one or more recombinant prokaryotic host cell strains including a genomic modification (e.g., deletions of genes rendering the genes inoperable) that preferably result in membrane extracts comprising sugar precursors for glycosylation at relatively high concentrations (e.g., in comparison to a strain not having the genomic modification). In some embodiments, one or more membrane extracts may be prepared from one or more recombinant prokaryotic host cell strains that have been modified to express one or more orthogonal or heterologous genes or gene clusters that are associated with glycoprotein synthesis. Preferably, the membrane extracts or mixed membrane extracts are enriched in glycosylation components, such as the one or more 4-epimerases, the one or more glycosyl-1-phosphate transferases, the one or more O-oligosaccharyltransferases, the one or more ß1,3-galactosyltransferase enzymes, the enzymes of an enzymatic pathway capable of producing cytidine-5′-monophosphate-5-N-acetylneuraminic acid (CMP-NeuNAc), and/or the sialyltransferases of the present disclosure.

### Methods For Producing O-Glycosylated Proteins

Another aspect of the present disclosure relates to a method for producing an O-glycosylated protein. This method involves providing a recombinant host cell expressing one more 4-epimerases, one or more glycosyl-1-phosphate transferases, one or more O-oligosaccharyltransferases, and a glycoprotein target comprising one or more serine and/or threonine residues. This method further involves culturing the host cell under conditions effective to: (i) produce N-acetylgalactosamine (GalNAc) linked to undecaprenyl pyrophosphate (Und-PP): and (ii) transfer the N-acetylgalactosamine (GalNAc) linked to undecaprenyl pyrophosphate (Und-PP) en bloc to a serine or threonine amino acid of the glycoprotein target.

Another aspect of the present disclosure relates to a method for producing an O-glycosylated protein. This method involves providing a recombinant host cell expressing one more 4-epimerases, one or more glycosyl-1-phosphate transferases, one or more O-oligosaccharyltransferases, one or more ß1,3-galactosyltransferase enzymes capable of transferring galactose (Gal) to undecaprenyl pyrophosphate (Und-PP)-linked N-Acetylgalactosamine (GalNAc), and a glycoprotein target comprising one or more serine and/or threonine residues. This method further involves culturing said host cell under conditions effective to: (i) produce N-acetylgalactosamine (GalNAc) linked to undecaprenyl pyrophosphate (Und-PP): (ii) extend Und-PP-GalNAc by a single galactose (Gal) monosaccharide to yield lipid-linked Gal-ß1,3-GalNAc; and (iii) transfer the lipid-linked Gal-ß1,3-GalNAc en bloc to a serine or threonine amino acid of the glycoprotein target.

Suitable host cells, 4-epimerases, glycosyl-1-phosphate transferases, O-oligosaccharyltransferases, and ß1,3-galactosyltransferase enzymes for use in the methods described herein are described in more detail supra.

In some embodiments, the host cell is an Escherichia coli cell. Exemplary suitable E. coli host cells according to the present disclosure include, without limitation, laboratory strains of E. coli selected from the group consisting of DH5α, NEB 10-beta, BL21(DE3), W3110, CLM24, CLM25, MC4100, MC4100, MCΔw, MCΔΔw, MCΔΔw-neuo-PS, MCΔΔwΔn-neuo-PS, and ZLKA (see, e.g., Table 5, infra).

In some embodiments, the 4-epimerase is a uridine diphosphate-N-acetylglucosamine (UDP-GlcNAc) 4-epimerase. For example, the 4-epimerase may be a Gne. The Gne may be C. jejuni Gne (c/Gne).

In some embodiments, the one or more glycosyl-1-phosphate transferases is a PglC. For example, the PglC is Acinetobacter baumannii ATCC 17978 PglC (AbPglC).

In some embodiments, the one or more O-oligosaccharyltransferases is PglL, a PglO, or a combination thereof. Thus, in some embodiments, the PglL is Neisseria meningitides PglL (NmPglL) and the PglO is Neisseria gonorrhoeae PglO (NgPglO).

In some embodiments, when the recombinant host cell expresses one or more ß1,3-galactosyltransferase enzymes capable of transferring galactose (Gal) to undecaprenyl pyrophosphate (Und-PP)-linked N-Acetylgalactosamine (GalNAc), the one or more ß1,3-galactosyltransferase enzymes capable of transferring galactose (Gal) to undecaprenyl pyrophosphate (Und-PP)-linked N-Acetylgalactosamine (GalNAc) is the ß-1,3-galactosyltransferase derived from the O-antigen biosynthesis pathway of enterohemorrhagic Escherichia coli O104 (EcWbwC).

In some embodiments of the methods disclosed herein, each of the one or more 4-epimerases, the one or more glycosyl-1-phosphate transferases, the one or more O-oligosaccharyltransferases, and the one or more ß1,3-galactosyltransferase enzymes capable of transferring galactose (Gal) to undecaprenyl pyrophosphate (Und-PP)-linked N-Acetylgalactosamine (GalNAc) are encoded by a polynucleotide sequence that is independently located either on an extrachromosomal plasmid carried by the prokaryotic host cell or in the recombinant prokaryotic host cell's genome.

In some embodiments, when the recombinant prokaryotic host cell does not express one or more ß1,3-galactosyltransferase enzymes capable of transferring galactose (Gal) to undecaprenyl pyrophosphate (Und-PP)-linked N-Acetylgalactosamine (GalNAc), the recombinant prokaryotic host cell further expresses: (i) the enzymes of an enzymatic pathway capable of producing cytidine-5′-monophosphate-5-N-acetylneuraminic acid (CMP-NeuNAc), and (ii) one or more α2,6-sialyltransferases. In accordance with such embodiments, the N-acetylgalactosamine (GalNAc) linked to undecaprenyl pyrophosphate (Und-PP) is extended by one or more sialic acid (NeuNAc) sugars before the transfer the N-acetylgalactosamine (GalNAc) linked to undecaprenyl pyrophosphate (Und-PP) en bloc to a serine or threonine amino acid of the glycoprotein target.

Suitable enzymes of an enzymatic pathway capable of producing cytidine-5′-monophosphate-5-N-acetylneuraminic acid (CMP-NeuNAc) are described in more detail supra and include, e.g., E. coli K1 NeuA, NeuB, NeuC, and NeuD.

Suitable α2,6-sialyltransferases are described in more detail supra and include, e.g., the α2,6-sialyltransferase from Photobacterium sp. JT-ISH-224.

In some embodiments, when the recombinant prokaryotic host cell expresses one or more ß1,3-galactosyltransferase enzymes capable of transferring galactose (Gal) to undecaprenyl pyrophosphate (Und-PP)-linked N-Acetylgalactosamine (GalNAc), the recombinant prokaryotic host cell further expresses: (i) the enzymes of an enzymatic pathway capable of producing cytidine-5′-monophosphate-5-N-acetylneuraminic acid (CMP-NeuNAc); and (ii) one or more α2,3-sialyltransferases and/or one or more α2,6-sialyltransferases; and wherein the lipid-linked Gal-ß1,3-GalNAc is extended by one or more sialic acid (NeuNAc) sugars before the transfer the lipid-linked Gal-ß1,3-GalNAc en bloc to a serine or threonine amino acid of the glycoprotein target.

Suitable enzymes of an enzymatic pathway capable of producing cytidine-5′-monophosphate-5-N-acetylneuraminic acid (CMP-NeuNAc) are described in more detail supra and include, e.g., E. coli K1 NeuA, NeuB, NeuC, and NeuD. Thus, in some embodiments, the enzymes of the enzymatic pathway capable of producing cytidine-5′-monophosphate-5-N-acetylneuraminic acid (CMP-NeuNAc) are encoded by the E. coli K1 neuDBAC genes.

Suitable α2,3-sialyltransferases and α2,6-sialyltransferases described in more detail supra. In some embodiments, the one or more α2,3-sialyltransferases is WbwA from E. coli O104 and the one or more α2,6-sialyltransferases is the α2,6-sialyltransferase from Photobacterium sp. JT-ISH-224.

Suitable glycoprotein targets are described in more detail supra.

Purified glycoproteins and/or glycoprotein reagents (e.g., one more 4-epimerase enzymes, one or more N,N′-diacetylbacilliosaminyl-1-phosphate transferase enzymes, one or more O-oligosaccharyltransferases, and one or more ß1,3-galactosyltransferase enzymes capable of transferring galactose (Gal) to undecaprenyl pyrophosphate (Und-PP)-linked N-Acetylgalactosamine (GalNAc)) may be obtained from the recombinant prokaryotic host cell described herein by several methods readily known in the art, including ion exchange chromatography, hydrophobic interaction chromatography, affinity chromatography, gel filtration, and reverse phase chromatography. In some embodiments, the glycoproteins and/or glycoprotein reagents described herein are produced in purified form (preferably at least about 70% pure, at least about 75% pure, at least about 80% pure, at least about 85% pure, at least about 90% pure, at least about 95% pure, at least about 96% pure, at least about 97% pure, at least about 98% pure, at least about 99% pure, at least about 99.5% pure, at least about 99.9% pure, or more) by conventional techniques.

Another aspect of the present disclosure relates to an in vitro method for producing an O-glycosylated protein. This method involves providing glycosylation reagents comprising one more 4-epimerases, one or more glycosyl-1-phosphate transferases, and one or more O-oligosaccharyltransferases; providing a glycoprotein target comprising one or more serine and/or threonine residues: and incubating said glycosylation reagents and said glycoprotein target under conditions effective to: (i) yield N-acetylgalactosamine (GalNAc) linked to undecaprenyl pyrophosphate (Und-PP), and (ii) transfer the N-acetylgalactosamine (GalNAc) linked to undecaprenyl pyrophosphate (Und-PP) en bloc to a serine or threonine amino acid of the glycoprotein target.

Another aspect of the present disclosure relates to an in vitro method for producing an O-glycosylated protein. This method involves providing glycosylation reagents comprising one more 4-epimerase enzymes, one or more heterologous N,N′-diacetylbacilliosaminyl-1-phosphate transferase enzymes, one or more heterologous O-oligosaccharyltransferases, and one or more ß1,3-galactosyltransferase enzymes capable of transferring galactose (Gal) to undecaprenyl pyrophosphate (Und-PP)-linked N-Acetylgalactosamine (GalNAc): providing a glycoprotein target comprising one or more serine and/or threonine residues; and incubating said glycosylation reagents and said glycoprotein target under conditions effective to: (1) yield lipid-linked Gal-ß1,3-GalNAc, and (ii) transfer the lipid-linked Gal-ß1,3-GalNAc and any additional sugars en bloc to a serine or threonine amino acid of the glycoprotein target.

In some embodiments, the glycosylation reagents are provided as a membrane extract of a recombinant prokaryotic host cell of the present disclosure. In some embodiments, the recombinant prokaryotic host cell is an Escherichia coli cell.

In some embodiments, the glycosylation reagents are provided in the form of purified enzymes.

Suitable 4-epimerases, glycosyl-1-phosphate transferases. O-oligosaccharyltransferases, and ß1,3-galactosyltransferase enzymes for use in the methods described herein are described in more detail supra.

In some embodiments, the 4-epimerase is a uridine diphosphate-N-acetylglucosamine (UDP-GlcNAc) 4-epimerase. For example, the 4-epimerase may be a Gne. The Gne may be C. jejuni Gne (CjGne).

In some embodiments, the one or more glycosyl-1-phosphate transferases is a PglC. For example, may be Acinetobacter baumannii ATCC 17978 PglC (AbPglC).

In some embodiments, the one or more O-oligosaccharyltransferases is PglL, a PglO, or a combination thereof. Thus, in some embodiments, the PglL is Neisseria meningitides PglL (NmPglL) and the PglO is Neisseria gonorrhoeae PglO (NgPglO).

In some embodiments, when the recombinant host cell expresses one or more ß1,3-galactosyltransferase enzymes capable of transferring galactose (Gal) to undecaprenyl pyrophosphate (Und-PP)-linked N-Acetylgalactosamine (GalNAc), the one or more ß1,3-galactosyltransferase enzymes capable of transferring galactose (Gal) to undecaprenyl pyrophosphate (Und-PP)-linked N-Acetylgalactosamine (GalNAc) is the ß-1,3; galactosyltransferase derived from the O-antigen biosynthesis pathway of enterohemorrhagic Escherichia coli O104 (EcWbwC).

In some embodiments of the methods disclosed herein, when the glycosylation reagents do not comprise one or more ß1,3-galactosyltransferase enzymes capable of transferring galactose (Gal) to undecaprenyl pyrophosphate (Und-PP)-linked N-Acetylgalactosamine (GalNAc), the glycosylation reagents further comprise: (i) the enzymes of an enzymatic pathway capable of producing cytidine-5′-monophosphate-5-N-acetylneuraminic acid (CMP-NeuNAc), and (ii) one or more α2,6-sialyltransferases. In accordance with such embodiments, the N-acetylgalactosamine (GalNAc) linked to undecaprenyl pyrophosphate (Und-PP) is extended by one or more sialic acid (NeuNAc) sugars before the transfer the N-acetylgalactosamine (GalNAc) linked to undecaprenyl pyrophosphate (Und-PP) en bloc to a serine or threonine amino acid of the glycoprotein target.

Suitable enzymes of an enzymatic pathway capable of producing cytidine-5′-monophosphate-5-N-acetylneuraminic acid (CMP-NeuNAc) are described in more detail supra and include, e.g., E. coli K1 NeuA, NeuB, NeuC, and NeuD.

Suitable α2,6-sialyltransferases are described in more detail supra and include, e.g., the α2,6-sialyltransferase from Photobacterium sp. JT-ISH-224.

In some embodiments, when the glycosylation reagents comprise one or more ß1,3-galactosyltransferase enzymes capable of transferring galactose (Gal) to undecaprenyl pyrophosphate (Und-PP)-linked N-Acetylgalactosamine (GalNAc), the reagents further comprise: (i) the enzymes of an enzymatic pathway capable of producing cytidine-5′-monophosphate-5-N-acetylneuraminic acid (CMP-NeuNAc); and (ii) one or more α2,3-sialyltransferases and/or one or more α2,6-sialyltransferases. In accordance with such embodiments, the lipid-linked Gal-ß1,3-GalNAc is extended by one or more sialic acid (NeuNAc) sugars before the transfer the lipid-linked Gal-ß1,3-GalNAc en bloc to a serine or threonine amino acid of the glycoprotein target.

Suitable enzymes of an enzymatic pathway capable of producing cytidine-5′-monophosphate-5-N-acetylneuraminic acid (CMP-NeuNAc) are described in more detail supra and include, e.g., E. coli K1 NeuA, NeuB, NeuC, and NeuD. Thus, in some embodiments, the enzymes of the enzymatic pathway capable of producing cytidine-5′-monophosphate-5-N-acetylneuraminic acid (CMP-NeuNAc) are encoded by the E. coli K1 neuDBAC genes.

Suitable α2,3-sialyltransferases and α2,6-sialyltransferases described in more detail supra. In some embodiments, the one or more α2,3-sialyltransferases is WbwA from E. coli O104 and the one or more α2,6-sialyltransferases is the α2,6-sialyltransferase from Photobacterium sp. JT-ISH-224.

Suitable glycoprotein targets are described in more detail supra.

Another aspect of the present disclosure relates to an in vitro method for producing an O-glycosylated protein. This method involves providing reagents suitable for synthesizing a glycoprotein target; providing glycosylation reagents comprising one more 4-epimerases, one or more glycosyl-1-phosphate transferases, and one or more O-oligosaccharyltransferases; providing a nucleic acid molecule encoding a glycoprotein target and incubating said reagents suitable for synthesizing a glycoprotein target, glycosylation reagents, and nucleic acid molecule encoding a glycoprotein target under conditions effective to: (i) synthesize the glycoprotein target encoded by the nucleic acid molecule encoding a glycoprotein target, (i) yield N-acetylgalactosamine (GalNAc) linked to undecaprenyl pyrophosphate (Und-PP), and (ii) transfer the N-acetylgalactosamine (GalNAc) linked to undecaprenyl pyrophosphate (Und-PP) en bloc to a serine or threonine amino acid of the glycoprotein target.

Another aspect of the present disclosure relates to an in vitro method for producing an O-glycosylated protein comprising. This method involves providing reagents suitable for synthesizing a glycoprotein target; providing glycosylation reagents comprising one more 4-epimerase enzymes, one or more heterologous N,N′-diacetylbacilliosaminyl-1-phosphate transferase enzymes, one or more heterologous O-oligosaccharyltransferases, and one or more ß1,3-galactosyltransferase enzymes capable of transferring galactose (Gal) to undecaprenyl pyrophosphate (Und-PP)-linked N-Acetylgalactosamine (GalNAc), providing a nucleic acid molecule encoding a glycoprotein target; and incubating said reagents suitable for synthesizing a glycoprotein target, glycosylation reagents, and nucleic acid molecule encoding a glycoprotein target under conditions effective to: (i) synthesize the glycoprotein target encoded by the nucleic acid molecule encoding a glycoprotein target, (ii) yield lipid-linked Gal-ß1,3-GalNAc, and (iii) transfer the lipid-linked Gal-ß1,3-GalNAc and any additional sugars en bloc to a serine or threonine amino acid of the glycoprotein target.

Reagents suitable for synthesizing a glycoprotein target are well known in the art and include, e.g., translation reagents.

Reagents for synthesizing proteins from a nucleic acid molecule and/or a recombinant genetic construct in vitro (i.e., in a cell-free environment) are well known in the art. These reagents or systems typically consist of extracts from rabbit reticulocytes, wheat germ, and E. coli. The extracts contain all the macromolecule components necessary for translation of an exogenous RNA molecule, including, for example, ribosomes, tRNAs, aminoacyl-tRNA synthetases, initiation, elongation, and termination factors. The other required components of the system include amino acids, energy sources (e.g., ATP, GTP), energy regenerating systems (e.g., creatine phosphate and creatine phosphokinase for eukaryote systems, and phosphoenol pyruvate and pyruvate kinase for prokaryote systems), and other cofactors (e.g., Mg.sup.2+, K.sup.+, etc.). If the nucleic acid molecule and/or recombinant genetic construct encoding the glycoprotein target is a DNA molecule, the cell-free translation reaction is coupled or linked to an initial transcription reaction that utilizes a RNA polymerase.

Suitable 4-epimerases, glycosyl-1-phosphate transferases, O-oligosaccharyltransferases, and ß1,3-galactosyltransferase enzymes for use in the methods described herein are described in more detail supra.

In some embodiments, the 4-epimerase is a uridine diphosphate-N-acetylglucosamine (UDP-GlcNAc) 4-epimerase. For example, the 4-epimerase may be a Gne. The Gne may be C. jejuni Gne (CjGne).

In some embodiments, the one or more glycosyl-1-phosphate transferases is a PglC. For example, may be Acinetobacter baumannii ATCC 17978 PglC (AbPglC).

In some embodiments, the one or more O-oligosaccharyltransferases is PglL, a PglO, or a combination thereof. Thus, in some embodiments, the PglL is Neisseria meningitides PglL (NmPglL) and the PglO is Neisseria gonorrhoeae PglO (NgPglO).

In some embodiments, when the recombinant host cell expresses one or more ß1,3-galactosyltransferase enzymes capable of transferring galactose (Gal) to undecaprenyl pyrophosphate (Und-PP)-linked N-Acetylgalactosamine (GalNAc), the one or more ß1,3-galactosyltransferase enzymes capable of transferring galactose (Gal) to undecaprenyl pyrophosphate (Und-PP)-linked N-Acetylgalactosamine (GalNAc) is the ß-1,3,-galactosyltransferase derived from the O-antigen biosynthesis pathway of enterohemorrhagic Escherichia coli O104 (EcWbwC).

Suitable glycoprotein targets are described in more detail supra.

In some embodiments, when the glycosylation reagents do not comprise one or more ß1,3-galactosyltransferase enzymes capable of transferring galactose (Gal) to undecaprenyl pyrophosphate (Und-PP)-linked N-Acetylgalactosamine (GalNAc), the glycosylation reagents may further comprise: (i) the enzymes of an enzymatic pathway capable of producing cytidine-5′-monophosphate-5-N-acetylneuraminic acid (CMP-NeuNAc); and (ii) one or more α2,6-sialyltransferases, and the N-acetylgalactosamine (GalNAc) linked to undecaprenyl pyrophosphate (Und-PP) is extended by one or more sialic acid (NeuNAc) sugars before the transfer of the N-acetylgalactosamine (GalNAc) linked to undecaprenyl pyrophosphate (Und-PP) en bloc to a serine or threonine amino acid of the glycoprotein target.

Suitable enzymes of an enzymatic pathway capable of producing cytidine-5′-monophosphate-5-N-acetylneuraminic acid (CMP-NeuNAc) are described in more detail supra and include, e.g., E. coli K1 NeuA, NeuB, NeuC, and NeuD.

Suitable α2,6-sialyltransferases are described in more detail supra and include, e.g., the α2,6-sialyltransferase from Photobacterium sp. JT-ISH-224.

In some embodiments, when the glycosylation reagents comprise one or more ß1,3-galactosyltransferase enzymes capable of transferring galactose (Gal) to undecaprenyl pyrophosphate (Und-PP)-linked N-Acetylgalactosamine (GalNAc), the glycosylation reagents may further comprise: (i) the enzymes of an enzymatic pathway capable of producing cytidine-5′-monophosphate-5-N-acetylneuraminic acid (CMP-NeuNAc); and (ii) one or more α2,3-sialyltransferases and/or one or more α2,6-sialyltransferases; and wherein the lipid-linked Gal-ß1,3-GalNAc is extended by one or more sialic acid (NeuNAc) sugars before the transfer the lipid-linked Gal-ß1,3-GalNAc en bloc to a serine or threonine amino acid of the glycoprotein target.

Suitable enzymes of an enzymatic pathway capable of producing cytidine-5′-monophosphate-5-N-acetylneuraminic acid (CMP-NeuNAc) are described in more detail supra and include, e.g., E. coli K1 NeuA, NeuB, NeuC, and NeuD. Thus, in some embodiments, the enzymes of the enzymatic pathway capable of producing cytidine-5′-monophosphate-5-N-acetylneuraminic acid (CMP-NeuNAc) are encoded by the E. coli K1 neuDBAC genes.

Suitable α2,3-sialyltransferases and α2,6-sialyltransferases described in more detail supra. In some embodiments, the one or more α2,3-sialyltransferases is WbwA from E. coli O104 and the one or more α2,6-sialyltransferases is the α2,6-sialyltransferase from Photobacterium sp. JT-ISH-224.

### Methods for Producing Lipid-Linked T Antigen

Another aspect of the present disclosure relates to a method for producing a lipid linked Gal-β1,3-GalNAcα (T antigen or core 1). This method involves providing a recombinant host cell expressing one or more 4-epimerases, one or more glycosyl-1-phosphate transferases, one or more ß1,3-galactosyltransferase enzymes capable of transferring galactose (Gal) to undecaprenyl pyrophosphate (Und-PP)-linked N-Acetylgalactosamine (GalNAc), and one or more O-Antigen ligases (e.g., EcWaaL). This method further involves culturing the host cell under conditions effective to: (i) produce Gal-ß1,3-GalNAc linked to undecaprenyl pyrophosphate (Und-PP) and (ii) transfer Gal-ß1,3-GalNAc linked to undecaprenyl pyrophosphate (Und-PP) en bloc to a lipid target (see, e.g., FIG. 8A).

Suitable recombinant host cells for use in the methods described herein are described in more detail supra. In some embodiments, the recombinant prokaryotic host cell is an Escherichia coli cell.

Suitable lipid targets include, e.g., lipid A core.

Suitable 4-epimerases, glycosyl-1-phosphate transferases, and ß1,3-galactosyltransferase enzymes for use in the methods described herein are described in more detail supra.

In some embodiments, the 4-epimerase is a uridine diphosphate-N-acetylglucosamine (UDP-GlcNAc) 4-epimerase. For example, the 4-epimerase may be a Gne. The Gne may be C. jejuni Gne (CjGne).

In some embodiments, the one or more glycosyl-1-phosphate transferases is a PglC. For example, may be Acinetobacter baumannii ATCC 17978 PglC (AbPglC).

In some embodiments, the one or more ß1,3-galactosyltransferase enzymes capable of transferring galactose (Gal) to undecaprenyl pyrophosphate (Und-PP)-linked N-Acetylgalactosamine (GalNAc) is the ß-1,3; galactosyltransferase derived from the O-antigen biosynthesis pathway of enterohemorrhagic Escherichia coli O104 (EcWbwC).

In some embodiments, the one or more O-Antigen ligases (e.g., EcWaaL) for use in the methods described herein is endogenous to the prokaryotic host cell. In other embodiments, the one or more O-Antigen ligases (e.g., EcWaaL) for use in the methods described herein is heterologous to the host cell.

Each of the 4-epimerases, glycosyl-1-phosphate transferases, and ß1,3-galactosyltransferase enzymes for use in the methods described herein may be heterologous to the prokaryotic host cell.

In some embodiments according to this aspect of the invention, the prokaryotic host cell does not encode an O-oligosaccharyltransferase.

In some embodiments, the recombinant host cell further expresses: (i) the enzymes of an enzymatic pathway capable of producing cytidine-5′-monophosphate-5-N-acetylneuraminic acid (CMP-NeuNAc); and (ii) one or more α2,3-sialyltransferases and/or one or more α2,6-sialyltransferases. In accordance with such embodiments, the lipid-linked Gal-ß1,3-GalNAc is extended by one or more sialic acid (NeuNAc) sugars before the transfer the lipid-linked Gal-ß1,3-GalNAc en bloc to the lipid target.

Suitable enzymes of an enzymatic pathway capable of producing cytidine-5′-monophosphate-5-N-acetylneuraminic acid (CMP-NeuNAc) are described in more detail supra and include, e.g., E. coli K1 NeuA, NeuB, NeuC, and NeuD. Thus, in some embodiments, the enzymes of the enzymatic pathway capable of producing cytidine-5′-monophosphate-5-N-acetylneuraminic acid (CMP-NeuNAc) are encoded by the E. coli K1 neuDBAC genes.

In some embodiments of the methods described herein, the enzymes of the enzymatic pathway capable of producing cytidine-5′-monophosphate-5-N-acetylneuraminic acid (CMP-NeuNAc) are heterologous to the prokaryotic host cell.

Suitable α2,3-sialyltransferases and α2,6-sialyltransferases described in more detail supra. In some embodiments, the one or more α2,3-sialyltransferases is WbwA from E. coli O104 and the one or more α2,6-sialyltransferases is the α2,6-sialyltransferase from Photobacterium sp. JT-ISH-224.

In some embodiments of the methods described herein, the one or more α2,3-sialyltransferases and/or the one or more α2,6-sialyltransferases are heterologous to the prokaryotic host cell.

### EXAMPLES

The examples below are intended to exemplify the practice of embodiments of the disclosure but are by no means intended to limit the scope thereof.

### Materials and Methods for Examples 1-6

### Bacterial Strains and Growth Conditions

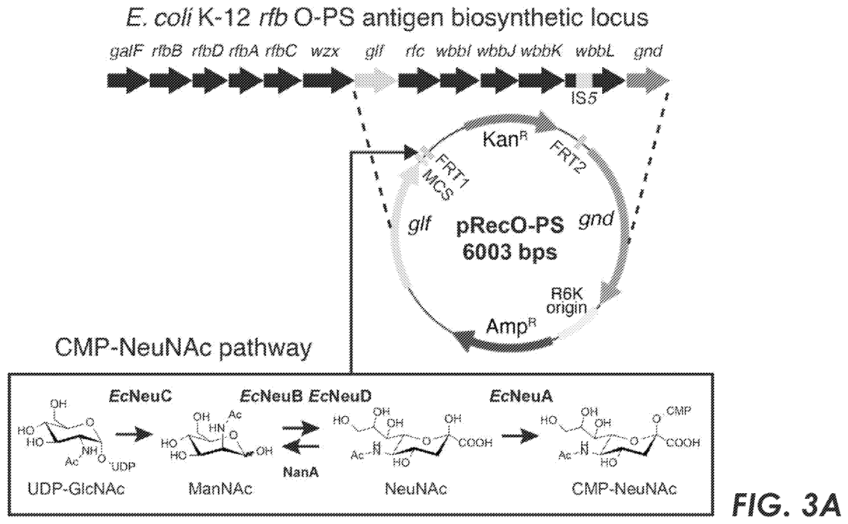

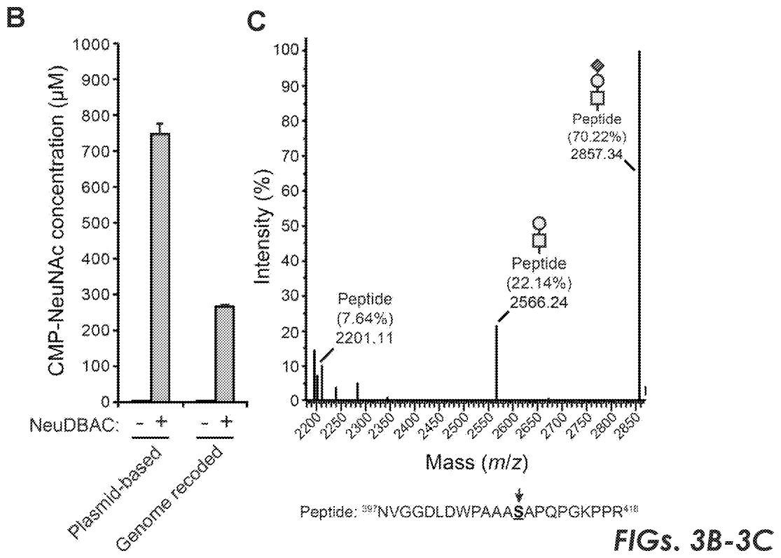

All strains used in the study are listed in Table 5 below. E. coli strain DH5a and NEB 10-beta were used for cloning and maintenance of plasmids while BL21(DE3) was used to produce purified acceptor proteins for 1VG reactions. Unless otherwise noted, strain CLM25 was used for all O-glycoprotein expression and was constructed by deleting wecA from CLM24 (Feldman et al., “Engineering N-linked Protein Glycosylation with Diverse O Antigen Lipopolysaccharide Structures in Escherichia coli,” Proc. Natl. Acad Sci. USA 102:3016-3021 (2005), which is hereby incorporated by reference in its entirety) through Plvir phage transduction where strain JW3758-2(Δrfe-735::kan) from the Keio collection (Baba et al., “Construction of Escherichia coli K-12 in-frame, Single-gene Knockout Mutants: The Keio Collection,” Mol. Syst Biol. 2:2006.0008 (2006), which is hereby incorporated by reference in its entirety) was used as the donor. MC4100 ΔwecA (MCΔw) and MC4100 ΔwecA ΔwaaL (MCΔΔw) were used as the hosts for flow cytometry screening and glyco-recoding to introduce the CMP-NeuNAc biosynthesis pathway. Strain MCΔw was generated by Plvir phage transduction of strain MC4100 to delete wecA using JW3758-2(Δrfe-735::kan) as the donor. Subsequent Plvir phage transduction of MCΔw to delete wool., using JW3597-1 (ΔrfaL734::kan) as donor yielded strain MCΔΔw. In all cases, after each deletion the linked kanamycin resistance (Kan^{R}) cassette was removed by transformation with the temperature-sensitive plasmid pCP20 as described in detail elsewhere (Datsenko K A & Wanner B L, “One-step Inactivation of Chromosomal Genes in Escherichia coli K-12 Using PCR Products,” Proc. Natl. Acad Sci. USA 97:6640-6645 (2000), which is hereby incorporated by reference in its entirety). The E. coli K1 neuDBAC genes encoding the CMP-NeuNAc biosynthesis pathway (Valentine et al., “Immunization with Outer Membrane Vesicles Displaying Designer Glycotopes Yields Class-switched, Glycan-specific Antibodies,” Cell. Chem. Biol. 23:655-665 (2016), which is hereby incorporated by reference in its entirety) were integrated into the chromosome of MCΔΔw using a previously described glyco-recoding strategy (Yates et al., “Glyco-recoded Escherichia coli: Recombineering-based Genome Editing of Native Polysaccharide Biosynthesis Gene Clusters,” Metab. Eng. 53:59-68 (2019), which is hereby incorporated by reference in its entirety). Briefly, the neuDBAC gene cluster was cloned into the pRecO-PS shuttle vector, which is uniquely designed to promote homologous recombination-based insertion of genes-of-interest in place of the existing genomic locus encoding the O-PS biosynthetic pathway between the glf and gnd genes (FIG. 3A). Next, the MCΔΔw strain carrying plasmid pKD46 encoding the λ-red recombinase was rendered electrocompetent and subsequently transformed with a linear PCR product derived from the pRecO-PSneuDBAC shuttle vector, which included the neuDBAC genes, the Kan^{R} cassette, and the flanking glf and gnd genes. A kanamycin-resistant chromosomal integrant was then chosen and the Kan^{R} marker was removed using the temperature-sensitive pE-FLP plasmid expressing the FLP recombinase, yielding strain MCΔΔw-neuo-PS. Finally, the genomic copy of nanA encoding the N-acetylneuraminate lyase involved in the catabolism of NeuNAc was deleted by Plvir phage transduction using Keio strain JW3194-1 (ΔnanA753::kan) as donor to create strain MCΔΔwΔn-neuo-PS. For extracellular secretion of O-glycoproteins, a secretion-optimized derivative of CLM24 was generated by deleting the yaiW gene (Natarajan et al., “An Engineered Survival-selection Assay for Extracellular Protein Expression Uncovers Hypersecretory Phenotypes in Escherichia coli,” ACS Synth. Biol. 6:875-883 (2017), which is hereby incorporated by reference in its entirety) by Plvir phage transduction using Keio strain JW0369 (ΔyaiW743::kan) as donor.

TABLE 5
Bacterial Strains and Plasmids Used in This Study

| | | |
|---|---|---|
| Strain or plasmid | Relevant genotype | Reference* |

E. coli strains

| | | |
|---|---|---|
| DH5α | F⁻ φ80lacZΔM15 Δ(lacZYA-argF)U169 deoR recA1 | Laboratory |
| | endA1 hsdR17 (rₖ⁻, mₖ⁺) gal-phoA supE44 λ⁻ thi-1 | stock |
| | gyrA96 relA1 | |
| NEB 10-beta | araD139 Δ(ara-leu)7697 fhuA lacX74 galK (f80 | New England |
| | Δ(lacZ)M15) mcrA galU recA1 endA1 nupG rpsL | Biolabs |
| | (Str^{R}) Δ(mrr-hsdRMS-mcrBC) | |
| BL21(DE3) | F⁻ ompT hsdS_{B} (r_{B}⁻, m_{B}⁻) gal dem (DE3) | Laboratory |
| | | Stock |
| W3110 | F⁻ λ⁻ rph-1 IN(rrnD-rrnE)1 | Laboratory |
| | | Stock |
| CLM24 | W3110 ΔwaaL | [1] |
| CLM25 | CLM24 ΔwecA | Described |
| | | herein |
| MC4100 | F⁻ araD139 Δ(argF-lac)169 λ⁻ e14⁻ flhD5301 | Laboratory |
| | Δ(fruKyeiR)725(fruA25) relA1 rpsL150(Str^{R}) | Stock |
| | rbsR22 Δ{fimBfimE)632(::IS1} deoC1 | |
| MCΔw | MC4100 ΔwecA | Described |
| | | herein |
| MCΔΔw | MC4100 ΔwecA ΔwaaL | Described |
| | | herein |
| MCΔΔw-neuo-_{PS} | MC4100 ΔwecA ΔwaaL with neuDBAC genes at O- | Described |
| | PS site | herein |
| MCΔΔwΔn-neuo-_{PS} | MCΔΔw-neuo-_{PS} ΔnanA | Described |
| | | herein |
| ZLKA | DH1 lacZ lacA nanKETA | [2] |

Plasmids

| | | |
|---|---|---|
| pMW07 | Yeast-based recombineering plasmid with yeast | [3] |
| | origin of replication and URA3 selection marker; | |
| | Cm^{R} | |
| pMW08 | Modified plasmid pMW07 with the yeast origin of | Described |
| | replication and URA3 selection marker removed; | herein |
| | Cm^{R} | |
| pOG-Tn | Genes encoding C. jejuni Gne and A. baumanii PglC | Described |
| | cloned in plasmid pMW08; Cm^{R} | herein |
| pOG-Tn- | Gene encoding glycoprotein-N-acetylgalactosamine | Described |
| HsC1GalT1 | 3-βgalactosyltransferase 1 from H. sapiens cloned | herein |
| | without the first 29 amino acids in plasmid pOG-Tn; | |
| | Cm^{R} | |
| pOG-Tn- | Gene encoding glycoprotein-N-acetylgalactosamine | Described |
| DmC1GalT2 | 3-βgalactosyltransferase A, isoform B from D. | herein |
| | melanogaster cloned without the first 50 amino | |
| | acids in plasmid pOG-Tn; Cm^{R} | |
| pOG-Tn- | Gene encoding D-galactosyl-β1-3-N-acetyl-D- | Described |
| BiGalHexNAcP | hexosamine phosphorylase from B. longum | herein |
| | subspecies infantis cloned in plasmid pOG-Tn; Cm^{R} | |
| pOG-Tn-CjCgtB | Gene encoding S42 mutant of β1-3- | Described |
| | galactosyltransferase from C. jejuni cloned in | herein |
| | plasmid pOG-Tn; Cm^{R} | |
| pOG-Tn-EcWbnJ | Gene encoding 1,3-α-N-acetylgalactosamine- | Described |
| | diphosphoundecaprenol β-1,3-galactosyltransferase | herein |
| | from E. coli O86 cloned in plasmid pOG-Tn; Cm^{R} | |
| pOG-Tn-EcWbwC | Gene encoding N-acetylgalactosamine-diphospho- | Described |
| | undecaprenol β1,3-galactosyltransferases from E. | herein |
| | coli O104 cloned in plasmid pOG-Tn; Cm^{R} | |
| pOG-TΔgne | Same as pOG-Tn-EcWbwC but lacking CjGne | Described |
| | epimerase; Cm^{R} | herein |
| pOG-Tn-NgPglO | Genes encoding C. jejuni Gne, A. baumanii PglC, | Described |
| | and N. gonorrhea PglO in plasmid pMW07; Cm^{R} | herein |
| pOG-Tn-NmPglL | Genes encoding C. jejuni Gne, A. baumanii PglC, | Described |
| | and N. meningitidis PglL in plasmid pMW07; Cm^{R} | herein |
| pOG-T | Genes encoding C. jejuni Gne, A. baumanii PglC, | Described |
| | and E. coli 0104 WbwC in plasmid pMW07; Cm^{R} | herein |
| pOG-T-NgPglO | Gene encoding PglO from N. gonorrhea in plasmid | Described |
| | pOG-T; Cm^{R} | herein |
| pOG-T-NmPglL | Gene encoding PglL from N. meningitidis in plasmid | Described |
| | pOG-T; Cm^{R} | herein |
| pCP20 | Plasmid encoding the FLP recombinase; | [4] |
| | temperature-sensitive replication and thermal | |
| | induction of FLP synthesis; Amp^{R}, Cm^{R} | |
| pKD46 | Plasmid encoding the λ-red recombinase; Amp^{R} | [5] |
| pE-FLP | Plasmid encoding the FLP recombinase; Amp^{R} | [6] |
| pRecO-PS | Shuttle vector for integration into the O-PS locus of | [7] |
| | E. coli K12 strains; Amp^{R}, Kan^{R} | |
| pRecO- | E. coli K1 neuDBAC genes cloned into pRecO-PS; | Described |
| PSneuDBAC | Amp^{R}, Kan^{R} | herein |
| pMLBy | pMLBAD vector with yeast origin of replication and | Laboratory |
| | URA3 selection marker: Tmp^{R} | stock |
| pConNeuDBAC | Plasmid encoding the E. coli K1 neuDBAC genes in | Described |
| | plasmid pMLBy with the araC gene and pBAD | herein |
| | promoter replaced with the J23100 constitutive | |
| | promoter from the Anderson library; Tmp^{R} | |
| pEXT20 | IPTG-inducible expression vector; Ap^{R} | [8] |
| pEXT-spDsbA- | Gene encoding E. coli maltose-binding protein | Described |
| MBP^{MOOR} | (MBP) with an E. coli DsbA signal peptide in place | herein |
| | of its native signal peptide and a C-terminal fusion | |
| | bearing the 25-residue MOOR sequence in plasmid | |
| | pEXT20: ApR | |
| pEXT-spDsbA- | Gene encoding E. coli MBP with an E. coli DsbA | Described |
| MBP^{MOORmut} | signal peptide in place of the native signal peptide | herein |
| | and a C-terminal fusion bearing the 25-residue | |
| | MOOR sequence with a Ser-to-Gly mutation in | |
| | plasmid pEXT20; Ap^{R} | |
| pEXT-spDsbA - | Same as pEXT-spDsbA -MBP^{MOOR} but with | Described |
| MBP^{MOOR}_ | sialyltransferase EcWbwA cloned in tandem; Ap^{R} | herein |
| EcWbwA | | |
| pEXT-spDsbA- | Same as pEXT-spDsbA-MBPMOOR but with | Described |
| MBP^{MOOR}-PspST6 | sialyltransferase PspST6 cloned in tandem; Ap^{R} | herein |
| pEXT-spDsbA- | Gene encoding E. coli YebF with an E. coli DsbA | Described |
| YebF-MBP^{MOOR} | signal peptide in place of its native signal peptide | herein |
| | and a C terminal fission with MBP and the 25- | |
| | residue MOOR sequence in plasmid pEXT20; Ap^{R} | |
| pEXT-spDsbA- | Gene encoding E. coli glutathione-S-transferase | Described |
| GST^{MOOR} | (GST) with an E. coli DsbA signal peptide and a C | herein |
| | terminal fusion bearing the 25-residue MOOR. | |
| | sequence in plasmid pEXT20; Ap^{R} | |
| pEXT-spDsbA- | Gene encoding single-chain Fv (scFv) antibody | Described |
| scFv13-R4^{MOOR} | fragment specific for E. coli β-galactosidase with an | herein |
| | E. coli DsbA signal peptide and a C terminal fusion | |
| | bearing the 25-residue MOOR sequence in plasmid | |
| | pEXT20; Ap^{R} | |
| pEXT-spDsbA- | Gene encoding superfolder green fluorescent protein | Described |
| sfGFP^{MOOR} | (sfGFP) with an E. coli DsbA signal peptide and a C | herein |
| | terminal fusion bearing the 25-residue MOOR | |
| | sequence in plasmid pEXT20; Ap^{R} | |
| pEXT-spDsbA- | Gene encoding sfGFP with an E. coli DsbA signal | Described |
| sfGFP^{Q157-MOOR} | peptide and an 25-residue MOOR sequence | herein |
| | internally grafted at position Q157 in plasmid | |
| | pEXT20; Ap^{R} | |
| pEXT-spDsbA- | Gene encoding cross-reacting material .197 | Described |
| CRM197^{MOOR} | (CRM197) with an E. coli DsbA signal peptide and | herein |
| | a C terminal fusion bearing the 25- residue MOOR | |
| | sequence in plasmid pEXT20; Ap^{R} | |
| pEXT-spDsbA- | Gene encoding the Haemophilus influenzae Protein | Described |
| PD^{MOOR} | D (PD) with an E. coli DsbA signal peptide and a C | herein |
| | terminal fusion bearing the 25- residue MOOR | |
| | sequence in plasmid pEXT20; Ap^{R} | |
| pEXT-spDsbA- | Same as pEXT-spDsbA-MBP^{MOOR} but with 8- | Described |
| MBP^{EPO} | residue motif derived from human erythropoietin in | herein |
| | place of the MOOR core sequence (WPAAASAP; | |
| | (SEQ ID NO: 24); Ap^{R} | |
| pEXT-spDsbA- | Same as pEXT-spDsbA-MBP^{MOOR} but with 8- | Described |
| MBP^{GPC} | residue motif derived from human glycophorin C in | herein |
| | place of the MOOR core sequence; Ap^{R} | |
| pEXT-spDsbA- | Same as pEXT-spDsbA-MBPMOOR but with 9- | Described |
| MBP^{SAP} | residue synthetic “SAP” motif (SAPSAPSAP) in | herein |
| | place of the MOOR core sequence; Ap^{R} | |
| pEXT-spDsbA- | Same as pEXT-spDsbA-MBP^{MOOR} but with 8- | Described |
| MBP^{MUC1}_8 | residue motif derived from human MUC1 in place of | herein |
| | the MOOR core sequence; Ap^{R} | |
| pEXT-spDsbA- | Same as pEXT-spDsbA-MBP^{MOOR} but with 12- | Described |
| MBP^{MUC1}_12 | residue motif derived from human MUC1 in place of | herein |
| | the MOOR core sequence; Ap^{R} | |
| pEXT-spDsbA- | Same as pEXT-spDsbA-MBP^{MOOR} but with 16- | Described |
| MBP^{MUC1}_16 | residue motif derived from human MUC1 in place of | herein |
| | the MOOR core sequence; Ap^{R} | |
| pEXT-spDsbA- | Same as pEXT-spDsbA-MBP^{MOOR} but with 20- | Described |
| MBP^{MUC1}_20 | residue motif derived from human MUC1 in place of | herein |
| | the MOOR core sequence; Ap^{R} | |
| pEXT-spDsbA- | Same as pEXT-spDsbA-MBP^{MOOR} but with 24- | Described |
| MBP^{MUC1}_24 | residue motif derived from human MUC1 in place of | herein |
| | the MOOR core sequence; Ap^{R} | |
| pEXT-spDsbA- | Same as pEXT-spDsbA-MBP^{MOOR} but with 41- | Described |
| MBP^{MUC1}_41 | residue motif derived from human MUCI in place of | herein |
| | the entire MOOR; Ap^{R} | |
| PVITRO1- | Genes encoding HER2/neu receptor-specific | Addgene |
| Trastuzumab- | humanized IgG1/κ antibody isotype cloned in | plasmid #61883 |
| IgG1/κ | plasmid pVITRO1; Hyg^{R} | |
| pVITRO1-5E5- | Genes encoding Tn-MUCI-specific chimeric IgG1/κ | Described |
| IgG1/κ | antibody isotype cloned in plasmid pVITRO1; Hyg^{R} | herein |
| pJL1-MBP^{MOOR} | Gene encoding MBP^{MOOR} in plasmid pJL1; Kan^{R} | Described |
| | | herein |
| pJLI-MBP^{MOORmut} | Gene encoding MBP^{MOORmut} in plasmid pJL1; Kan^{R} | Described |
| | | herein |

*[1] Feldman et al., “Engineering N-Linked Protein Glycosylation with Diverse O Antigen Lipopolysaccharide Structures in Escherichia coli,” Proc. Natl. Acad. Sci. USA 102:3016-3021 (2205);
[2] Fierfort & Samain, “Genetic Engineering of Escherichia coli for the Economical Production of Sialylated Oligosaccharides.” J. Biotechnol. 134:261-265 (2008);
[3] Valderrama-Rincon et al., “An Engineered Eukaryotic Protein Glycosylation Pathway in Escherichia coli,” Nat. Chem. Biol. 8:434-436 (2012);
[4] Cherepanov & Wackeragel, “Gene Disruption in Escherichia coli: TeR and KmR Cassettes with the Option of Flpcatalyzed Excision of the Antibiotic-Resistance Determinant,” Gene 158:9-14 (1995);
[5] Datsenko & Wanner, “One-Step Inactivation of Chromosomal Genes in Escherichia coli K-12 using PCR Products,” Proc. Natl. Acad. Sci. USA 97:6640-6645 (2000);
[6] St-Pierre etal., “One-Step Cloning and Chromosomal Integration of DNA,” ACS Synth. Biol. 2:537-541 (2013);
[7] Yates et al., “Glyco-Recoded Escherichia coli: Recombineering-Based Genome Editing of Native Polysaccharide Biosynthesis Gene Clusters,” Metab. Eng. 53:59-68 (2019); and
[8] Dykxhoom et al., “A Set of Compatible Tac Promoter Expression Vectors,” Gene 177:133-136 (1996), which are hereby incorporated by reference in their entirety.

All cultures were grown at 37° C. in Luria-Bertani (LB) media containing D-glucose (0.2% w/v) as well as 20 μg/ml chloramphenicol (Cm), 100 μg/ml trimethoprim (Tmp), and 100 μg/ml ampicillin (Amp) as needed for plasmid maintenance. Induction of protein expression was always performed at mid-log phase (Abs₆₀₀˜0.6) with 0.1 mM isopropyl β-D-thiogalactoside (IPTG) and 0.2% (w/v) L-arabinose at 16° C. for 16-20 hours. For yield determination experiments, cells were grown in 100 ml of Terrific Broth (TB) at 37° C. until mid-log phase and then induced with 1 mM IPTG and 0.2% (w/v) L-arabinose at 16° C. for 22 hours. Following expression, cells were harvested and protein purification was performed as described below.

### Plasmid Construction

All plasmids used in the study are listed in Table 5. Plasmid construction was performed according to standard cloning protocols using restriction enzymes from New England Biolabs. The pOG backbones were cloned in either the yeast recombineering plasmid pMW07 (Valderrama-Rincon, “An Engineered Eukaryotic Protein Glycosylation Pathway in Escherichia coli.,” Nat. Chem. Biol. 8:434-436 (2012), which is hereby incorporated by reference in its entirety) or a modified derivative of pMW07, namely pMW08, in which the yeast origin of replication and URA3 gene were deleted. Plasmid pOG-Tn was generated by the Gibson assembly method. Briefly, the genes encoding CjGne and AbPglC were PCR amplified with overlapping regions, and subsequently cloned into pMW08 using the NEBuilder HiFi DNA Assembly Cloning Kit (New England Biolabs) to generate plasmid pOG-Tn. Each of the candidate GalT enzymes was cloned into pOG-Tn by first obtaining codon-optimized DNA corresponding to each GalT gene synthesized with overlapping regions to facilitate recombination (Twist Biosciences). These genes were then amplified by PCR and cloned into pOG-Tn by Gibson assembly. A similar strategy was followed to generate plasmid pOG-T. Briefly, the genes encoding CjGne, AbPglC, EcWbwC were PCR amplified with overlapping regions, and subsequently cloned into pMW07 using the NEBuilder HiFi DNA Assembly Cloning Kit (New England Biolabs) to generate the pOG-T. Genes encoding NgPglO and NmPglL were added to pOG-Tn and pOG-T as follows. First, codon-optimized DNA encoding the NgPglO and NmPglL genes was synthesized with overlapping regions to facilitate recombination (Twist Biosciences). The synthesized genes were then amplified by PCR to have overlapping ends and recombined with linearized versions of plasmids pOG-Tn and pOG-T using a modified “lazy bones” protocol (Shanks et al., “Saccharomyces Cerevisiae-based Molecular Tool Kit for Manipulation of Genes from Gram-negative Bacteria,” Appl. Environ. Microbiol. 72:5027-5036 (2006), which is hereby incorporated by reference in its entirety). Briefly, 0.5 ml of an overnight yeast culture was pelleted and washed in sterile TE buffer (10 mM Tris-HCl pH 8.0 and 1 mM EDTA). 0.4 mg of salmon sperm carrier DNA (Sigma), plasmid DNA, and PCR products were added to the pellet along with 0.5 ml lazy bones solution (40% polyethylene glycol MW 3350, 0.1 M lithium acetate, 10 mM Tris-HCl pH 7.5 and 1 mM EDTA). After vortexing for 1 minute, the solution was incubated up to 4 days at room temperature. Cells were heat-shocked at 42° C., pelleted, and plated on selective medium. Plasmids were isolated from individual transformants and confirmed by DNA sequencing.

All acceptor proteins were cloned in plasmid pEXT20 (Dykxhoorn et al., “A Set of Compatible Tac Promoter Expression Vectors,” Gene 177:133-136 (1996), which is hereby incorporated by reference in its entirety). Briefly, the gene encoding E. coli MBP lacking its native 26-residue signal peptide was PCR amplified with primers that introduced the N-terminal signal peptide from E. coli DsbA, which permits periplasmic localization and glycosylation of fused proteins (Fisher et al., “Production of Secretory and Extracellular N-linked Glycoproteins in Escherichia coli,” Appl. Environ. Microbiol. 77:871-881 (2011), which is hereby incorporated by reference in its entirety). The resulting PCR product was cloned into pEXT20 using restriction cloning between the EcoRI and XbaI sites. The MOOR tag was comprised of an 8-residue core sequence (WPAAASAP (SEQ ID NO: 24)) that mimics the S63 glycosite in pilin (PilE), one of the native substrates of NmPglL (Pan et al., “Biosynthesis of Conjugate Vaccines Using an O-Linked Glycosylation System,” MBio. 7:e00443-00416 (2016), which is hereby incorporated by reference in its entirety), as well as two hydrophilic flanking sequences (DPRNVGGDLD (SEQ ID NO: 27) and QPGKPPR (SEQ ID NO: 28)) that are required for glycosylation. This sequence was synthesized as a G block (Integrated DNA Technologies) with a hexa-histidine epitope tag at its C-terminus and cloned between the XbaI and HindIII sites. All other acceptor proteins including GST, scFv 13-R4, CRM 197, PD, YebF-MBP, sfGFP, and sfGFP^{Q157} were synthesized as G blocks (Integrated DNA Technologies) and cloned in place of MBP by Gibson assembly using the EcoRI and XbaI sites to linearize the backbone. All additional acceptor peptides including MOORmut, the 8-residue EPO sequence, the 8-residue GPC sequence, the 9-residue SAP sequence, the 8-residue MUC1 sequence (MUC1_8), MUC1_12, MUC1_16, MUC1_20, MUC1_24, and MUC1_41 were synthesized as G blocks (Integrated DNA Technologies) and cloned in place of the MOOR sequence at the C-terminus of MBP by Gibson assembly using the XbaI and HindIII sites to linearize the backbone. The MUC1 sequence designs included motifs based on the most frequent minimal epitopes of natural MUC1 IgG and IgM antibodies including PPAHGVT (SEQ ID NO: 29), PDTRP (SEQ ID NO: 30), and RPAPGS (SEQ ID NO: 31) (von Mensdorff-Pouilly et al., “Reactivity of Natural and Induced Human Antibodies to MUC1 Mucin with MUC1 Peptides and n-acetylgalactosamine (GalNAc) Peptides,” Int J Cancer 86:702-712 (2000), which is hereby incorporated by reference in its entirety) and in epitopes that bind to specific human MHC class I molecules including STAPPAHGV (SEQ ID NO: 32), SAPDTRPAP (SEQ ID NO: 33), TSAPDTRPA (SEQ ID NO: 34), and APDTRPAPG (SEQ ID NO: 35) (Apostolopoulos et al., “Induction of HLA-A2-restricted CTLs to the Mucin 1 Human Breast Cancer Antigen,” J. Immunol. 159:5211-5218 (1997), which is hereby incorporated by reference in its entirety). The sialyltransferase used to produce the ST antigen was cloned adjacent to spDsbA-MBP^{MOOR} in the pEXT20 acceptor plasmid. For sialylation of T antigen, E. coli O104 WbwA was acquired as a codon-optimized G block (Integrated DNA Technologies) and cloned downstream of spDsbA-MBP^{MOOR} in plasmid pEXT20-spDsbA-MBP^{MOOR} using Gibson assembly, yielding plasmid pEXT-spDsbA-MBP^{MOOR}-EcWbwA. For sialylation of Tn antigen, the gene encoding EcWbwA was replaced with α2,6-sialyltransferase from Photobacterium sp. JT-ISH-224, yielding plasmid pEXT-spDsbA-MBP^{MOOR}-PspST6. The plasmid for expression of the neuDBAC genes was constructed by yeast-based recombineering which involved cloning the E. coli K1 neuDBAC genes into plasmid pMLBy, which is a variant of plasmid pMLBAD that contains the yeast origin of replication and URA3 gene. The resulting plasmid was linearized with NheI after which the araC gene and pBAD promoter were replaced with the J23100 constitutive promoter from the Anderson library as described previously (Glasscock et al., “A Flow Cytometric Approach to Engineering Escherichia coli for Improved Eukaryotic Protein Glycosylation,” Metab. Eng. 47:488-495 (2018), which is hereby incorporated by reference in its entirety). The resulting pConNeuDBAC plasmid was used to transform strain ZLKA, a nanA-deficient host used previously for producing CMP-NeuNAc (Fierfort N & Samain E, “Genetic Engineering of Escherichia coli for the Economical Production of Sialylated Oligosaccharides,” J. Biotechnol. 134:261-265 (2008), which is hereby incorporated by reference in its entirety). Cell-free expression plasmids were generated by first PCR-amplifying the genes encoding MBP^{MOOR} and MBP^{MOORmut} from pEXT-spDsbA-MBP^{MOOR} and pEXT-spDsbA-MBP^{MOORmut}, respectively. The resulting PCR products were then ligated between NdeI and SalI restriction sites in plasmid pJL1, a pET-based vector used in cell-free glycoprotein synthesis reaction as described previously (Jaroentomeechai et al., “Single-pot Glycoprotein Biosynthesis Using a Cell-free Transcription-translation System Enriched with Glycosylation Machinery,” Nat Commun 9:2686 (2018), which is hereby incorporated by reference in its entirety).

Finally, a plasmid for expressing chimeric 5E5 antibody was constructed as described previously (Cox E C et al., “Antibody-mediated Endocytosis of Polysialic Acid Enables Intracellular Delivery and Cytotoxicity of a Glycan-directed Antibody-drug Conjugate,” Cancer Res. 79:1810-1821 (2019), which is hereby incorporated by reference in its entirety). First, DNA sequences for the V_{H} and V_{L} domains of mouse mAb 5E5 (Sorensen et al., “Chemoenzymatically Synthesized Multimeric Tn/STn MUC1 Glycopeptides Elicit Cancer-specific Anti-MUC1 Antibody Responses and Override Tolerance,” Glycobiology 16:96-107 (2006), which is hereby incorporated by reference in its entirety) were obtained from U.S. Pat. No. 10,189,908 B2 (which is hereby incorporated by reference in its entirety) and ordered as genes from GeneArt Gene Synthesis (Thermo Fisher). The 5E5 V_{H} and V_{L} sequences were then swapped with the existing variable region sequences in pVITRO1-Trastuzumab-IgG1/κ (Addgene plasmid #61883) to generate the vector pVITRO1-5E5-IgG1/κ according to previously published method (Dodev T S et al., “A Tool Kit for Rapid Cloning and Expression of Recombinant Antibodies,” Sci. Rep. 4:5885 (2014), which is hereby incorporated by reference in its entirety). All plasmids were confirmed by DNA sequencing.

### Immunoblot Analysis

Glycoprotein expression was carried out in 150-ml cultures for 16-20 hours. Cells were pelleted at 10,000×g for 30 minutes at 4° C., resuspended in 2 ml of lysis buffer containing 50 mM sodium phosphate, 300 mM sodium chloride, and 10 mM imidazole. Samples were frozen at −80° C. overnight. Cells were then thawed, gently agitated at room temperature with 200 μg/ml of lysozyme (Sigma) for 15 minutes, and lysed by sonication. Lysed samples were then centrifuged at 10,000×g for 30 minutes at 4° C., and the supernatant was subjected to Ni²⁺ affinity purification using Ni-NTA spin columns (Qiagen) according to the manufacturer's protocol. For preparation of extracellular culture supernatants, 10 ml of cells were pelleted by centrifugation at 10,000×g for 30 minutes. 5 ml of the cleared supernatant was then transferred to a fresh tube to which 5 ml of 20% chilled trichloroacetic acid was added. The mixture was vortexed and incubated at 4° C. without agitation for 16-20 hours. The sample was then centrifuged at 21,000×g for 30 minutes at 4° C. The supernatant was discarded and the pellet was resuspended in 1 ml of acetone. The sample was again centrifuged at 21,000×g for 30 minutes at 4° C., allowed to dry at 37° C. for 10 minutes, and resuspended in 60 μl of PBS.

Purified protein samples were prepared in Bolt LDS Sample Buffer (Thermo Fisher) and resolved on Bolt SDS-PAGE gels (Thermo Fisher). Following electrophoresis, proteins were transferred onto Immobilon-P polyvinylidene difluoride (PVDF) membranes (0.45 μm; Thermo Fisher) according to the manufacturer's protocol. Antibodies used included: HRP-conjugated anti-hexa-histidine polyclonal antibody (Abeam cat #ab 1187; dilution 1:5,000), mouse anti-human MUC1 antibody (BD Biosciences cat #555925; dilution 1:1,000), biotinylated PNA (Vector labs cat #B-1075; dilution 1:1,000), biotinylated VVA (Vector labs cat #B-1235; dilution 1:500), and chimeric 5E5 antibody (dilution 1:250). The latter antibody was produced in-house using FreeStyle™ 293-F cells (Thermo Fisher) transfected with pVITRO1-5E5-IgG1/κ and purified from cell culture supernatants using Protein A/G agarose (Thermo Fisher) according to the manufacturer's recommendations. Secondary antibodies included: HRP-conjugated rabbit anti-human IgG (Fc) antibody (Thermo Fisher cat #31423; 1:2,500 dilution) and HRP-conjugated goat anti-mouse IgG (H&L) antibody (Abeam cat #ab6789; 1:2,500 dilution). Biotinylated lectins were detected using HRP-conjugated Extravidin (Sigma cat #E2886; dilution 1:2,000). Detection of blots was performed using Bio-Rad enhanced chemiluminescent (ECL) substrate. All immunoblots were visualized using a Chemidoc XRS+ system with Image Lab software (Bio-Rad).

### Mass Spectrometry Analysis of Protein Glycosylation

All reagents were purchased from Sigma Aldrich unless otherwise mentioned. Proteins were separated on SDS-PAGE gels after which gel pieces containing the glycoprotein bands were excised, cut into small pieces of about 1 mm², and destained by treatment with 300 μL of a 1:1 mixture of acetonitrile and 50 mM aqueous NH₄HCO₃ followed by 500 μl of 100% acetonitrile. Since the glycoproteins did not have cysteine residues, reduction and alkylation was not performed. The glycoproteins were directly digested by adding 50 μl of digestion buffer with 12.5 μl of sequencing-grade trypsin (0.4 μg/μ1; Promega) to the gel pieces and incubating at 37° C. for 12 hours. The digested peptides were extracted twice by 5% formic acid in 200 μL of 1:2 water:acetonitrile and filtered through a 0.2-μm filter. The digests were then dried using a SpeedVac, and subsequently re-dissolved in solvent A (0.1% formic acid in water) and stored at −30° C. until analysis by nano-LC-MS/MS.

The digests were analyzed on an Orbitrap Fusion Tribrid mass spectrometer (Thermo Fisher) equipped with a nanospray ion source and connected to a Dionex binary solvent system. Pre-packed nano-LC columns of 15-cm length with 75-μm internal diameter (id), filled with 3-μm C18 material (reverse phase) were used for chromatographic separation of samples. The precursor ion scan was acquired at 120,000 resolution in the Orbitrap analyzer and precursors at a time frame of 3 seconds were selected for subsequent MS/MS fragmentation in the Orbitrap analyzer at 15,000 resolution or in ion trap. The threshold for triggering an MS/MS event with either higher-energy collisional dissociation product-triggered electron-transfer dissociation (HCDpdETD) program or electron-transfer dissociation (ETD) was set to 1,000 counts. Charge state screening was enabled, and precursors with unknown charge state or a charge state of +1 were excluded (positive ion mode). Dynamic exclusion was enabled (exclusion duration of 30 secs).

The LC-MS/MS spectra of tryptic digest of glycoproteins were searched against the respective fasta sequence of mucin fragment using Byonic™ software versions 3.2 and 3.5 with the specific cleavage option enabled, and selecting trypsin as the digestion enzyme. Oxidation of methionine, deamidation of asparagine and glutamine, and O-glycan masses of HexNAc (m/z 203.079). HexHexNAc (m/z 365.132), and NeuNAcHexHexNAc (m/z 656.228) were used as variable modifications. The LC-MS/MS spectra were also analyzed manually for the glycopeptides using Xcalibur 4.2 software. The HCDpdETD and ETD MS' spectra of glycopeptides were evaluated for the glycan neutral loss pattern, oxonium ions, and the glycopeptide fragmentations to assign the sequence and the presence of glycans in the glycopeptides. The peptide fragments at high resolution from ETD spectra were analyzed for the localization of O-glycosylation sites.

### Quantification of In Vivo CMP-NeuNAc Levels

For detection and quantification of nucleotide sugars, E. coli cells were pelleted to an equivalent to Abs₆₀₀ of ˜30, resuspended in 1 mL ultrapure water, and lysed by sonication. Following centrifugation at 30,000×g, the supernatant was collected and analyzed within 4 hours. Cleared E. coli lysates were diluted twofold in ultrapure water and injected into an UPLC-ESI-MS system (Waters) for analysis. The autosampler was set at 10° C. Separation was performed on an Acquity BEH C18 Column (1.7 μm, 2.1 mm×50 mm; Waters). The elution started from 95% mobile phase A (5 mM TBA aqueous solution, adjusted to pH 4.75 with acetic acid) and 5% mobile phase B (5 mM TBA in Acetonitrile), raised to 57% B in 2 minutes, further raised to 100% B in 0.5 minutes, and then held at 100% B for 2 minutes, and returned to initial conditions over 0.1 minute and held for 4 minutes to re-equilibrate the column. The flow rate was set at 0.6 ml/min with an injection volume of 2 μL. The column was preconditioned by pumping the starting mobile phase mixture for 10 minutes, followed by repeating twice the gradient protocol specified above prior to any injections. LC-ESI-MS chromatograms were acquired in negative ion mode under the following conditions: cpme voltage of 10 V, dry temperature at 520° C., and an acquisition range of m/z 400-900. Selected ion recordings were specified for CMP-NeuNAc. A standard curve was generated using commercial CMP-NeuNAc (CarboSynth).

### Flow Cytometric Analysis

To analyze the activity of candidate GalT enzymes, a flow cytometry-based screen was adapted from Glasscock et al., “A Flow Cytometric Approach to Engineering Escherichia coli for Improved Eukaryotic Protein Glycosylation,” Metab. Eng. 47:488-495 (2018), which is hereby incorporated by reference in its entirety). Briefly, overnight cultures of each strain were grown in LB with relevant antibiotics. Cells were subcultured to an Abs₆₀₀ of ˜0.1 in 10 ml LB and grown for 16-20 hours at 30° C. The next day, 1 ml of culture was washed twice with 1 ml PBS and resuspended in 500 μl PBS. All samples were diluted to an Abs₆₀₀ of ˜0.2 in 250 μl PBS. Detection of the disaccharide T antigen was performed with PNA-FITC conjugate (Vector labs cat #FL1071). PNA-FITC was diluted 1:500 in PBS and 250 μl of diluted lectin was added to cells, followed by incubation at 37° C. for 30 minutes. Cells were pelleted at 6,000×g for 4 minutes, washed in 1 ml PBS, resuspended in 1 ml PBS, and analyzed by flow cytometry using a FACSCalibur flow cytometer (BD Biosciences). All experiments were performed in triplicate with the resulting data generated through CellQuest Pro 6.0 and analyzed using FlowJo 10.5 software.

### Cell-Free O-Glycosylation Reactions

For IVG reactions, crude membrane extracts enriched with NgPglO and UndPP-linked T antigen was prepared as described previously (Jaroentomeechai et al., “Single-Pot Glycoprotein Biosynthesis Using a Cell-Free Transcription-Translation System Enriched with Glycosylation Machinery,” Nature Commun. 9:2686 (2018), which is hereby incorporated by reference in its entirety). Briefly, CLM25 cells carrying plasmid pOG-T NgPglO were grown for 16-20 hours at 37° C. in LB media. The following day, cells were subcultured into 4 L LB media and allowed to grow at 37° C. until mid-log phase (Abs₆₀₀˜0.0.6). Cells were then induced for 20 hours at 16° C. with 0.2% L-arabinose. Cells were harvested by centrifugation at 10,000×g for 30 minutes at 4° C., and then resuspended in buffer containing 50 mM Tris-HCl (pH 8.0) and 25 mM sodium chloride. Cells were lysed by passing the cell suspension through a high-pressure homogenizer (Avestin) five times and the resulting lysate was centrifuged at 15,000×g for 20 minutes at 4° C. The supernatant was collected and subjected to ultracentrifugation at 100,000×g for 2 hours at 4° C. The resulting pellet corresponding to the membrane fraction was collected and resuspended in 3 ml of buffer containing 50 mM Tris-HCl (pH 7.0), 25 mM sodium chloride, and 0.1% (w/v) n-dodecyl-β-D-maltoside (DDM). The resuspended pellet was incubated with mild agitation at room temperature for 1 hour to enable the solubilization of NgPglO and LLOs. Following incubation, the mixture was centrifuged at 16,000×g for 1 hour at 4° C., and the supernatant was retained as a crude membrane extract. In parallel, acceptor proteins MBP^{MOOR} and MBP^{MOORmut} were purified as described above from a 500-ml culture of BL21(DE3) cells carrying either pEXT-spDsbA-MBP^{MOOR} or pEXT-spDsbA-MBP^{MOORmut}. In vitro glycosylation of purified acceptor proteins was carried out in 1.5-ml reactions containing 50 μg of purified acceptor protein and 1 ml of crude membrane extract in reaction buffer containing 10 mM HEPES (pH 7.5), 10 mM manganese chloride, and 1% (w/v) DDM. The reaction was incubated at 30° C. for 16 hours with mild tumbling. Upon completion of the reaction, acceptor proteins were purified from the reaction mixture by standard Ni²⁺ affinity purification using Ni-NTA spin columns (Qiagen) followed by concentration of samples.

For single-pot CFGpS, crude S12 extracts enriched with NgPglO and UndPP-linked T antigen glycans were prepared as described previously (Jaroentomeechai et al., “Single-pot Glycoprotein Biosynthesis Using a Cell-free Transcription-translation System Enriched with Glycosylation Machinery,” Nat. Commun. 9:2686 (2018), which is hereby incorporated by reference in its entirety). Briefly, CLM25 cells carrying plasmid pOG-T-NgPglO were grown at 37° C. in 2×YTPG (10 g/L yeast extract, 16 g/L tryptone, 5 g/L NaCl, 7 g/L K₂HPO₄, 3 g/L KH₂PO₄, 18 g/L glucose, pH 7.2) until the Abs₆₀₀ reached ˜1. The culture was then induced with 0.02% (w/v) L-arabinose and the protein expression was allowed to proceed at 30° C. until the Abs₆₀₀ reached ˜3. All subsequent steps were carried out at 4° C. unless otherwise stated. Cells were harvested and washed twice using S12 buffer (10 mM tris acetate, 14 mM magnesium acetate, 60 mM potassium acetate, pH 8.2). The pellet was then resuspended in 1 ml per 1 g cells of S12 buffer. The resulting suspension was passed once through a EmulsiFlex-B15 high-pressure homogenizer (Avestin) at 20,000-25,000 psi to lyse cells. The extract was then centrifuged twice at 12,000×g for 30 minutes to remove cell debris and the supernatant was collected and incubated at 37° C. for 60 minutes. Following centrifugation at 15,000×g for 15 minutes at 4° C., the supernatant was collected, flash-frozen in liquid nitrogen, and stored at −80° C. CFGpS reactions were carried out at 1-ml reaction volumes in a 15-ml conical tube using a modified PANOx-SP system (Jewett M C & Swartz J R, “Mimicking the Escherichia coli Cytoplasmic Environment Activates Long-lived and Efficient Cell-free Protein Synthesis,” Biotechnol Bioeng 86:19-26 (2004), which is hereby incorporated by reference in its entirety). The reaction mixture contained the following components: 0.85 mM each of GTP, UTP, and CTP, 1.2 mM ATP, 34.0 μg/ml folinic acid, 170.0 μg/ml of E. coli tRNA mixture, 130 mM potassium glutamate, 10 mM ammonium glutamate, 12 mM magnesium glutamate, 2 mM each of 20 amino acids, 0.4 mM nicotinamide adenine dinucleotide (NAD), 0.27 mM coenzyme-A (CoA), 1.5 mM spermidine, 1 mM putrescine, 4 mM sodium oxalate, 33 mM phosphoenolpyruvate (PEP), 57 mM HEPES, 6.67 μg/ml plasmid, and 27% (v/v) of cell lysate. Protein synthesis was carried out for 30 minutes at 30° C., after which protein glycosylation was initiated by the addition of sucrose and tetracycline at the final concentration of 100 mM and 10 μg/ml, respectively, and carried out at 30° C. for 16 hours. To recover protein products, reaction mixtures were passed through a Ni-NTA spin column (Qiagen) twice, washed, and eluted with 300 mM imidazole. Samples were concentrated and analyzed by SDS-PAGE followed by immunoblotting analysis.

### Example 1—An Engineered Pathway for Tn Antigen Biosynthesis

The enable orthogonal O-glycosylation in E. coli required assembling an en bloc pathway for producing the simplest mucin-type O-glycoform, GalNAcα (Tn antigen) (FIGS. 1A-1B). First, to eliminate formation of Und-PP-GlcNAc, an unwanted precursor in the context of mucin-type O-glycosylation, the gene encoding the native E. coli phosphoglycosyltransferase WecA was deleted from the genome of strain CLM24. This new strain, called CLM25, also lacked the waaL gene encoding the O-antigen ligase, a deletion that makes Und-PP-linked glycans available for the O-OST by preventing their unwanted transfer to lipid A-core (Feldman et al., “Engineering N-Linked Protein Glycosylation with Diverse 0 Antigen Lipopolysaccharide Structures in Escherichia coli,” Proc. Natl. Acad Sci. USA 102:3016-3021 (2005), which is hereby incorporated by reference in its entirety). Next, a plasmid encoding the C. jejuni UDP-Glc(NAc) 4-epimerase (CjGne), which generates the activated sugar donor UDP-GalNAc from UDP-GlcNAc in the cytoplasm, was created. While a number of epimerase homologs were considered, CjGne was chosen because of its effectiveness in previous glycoengineering efforts (Mueller et al., “High Level In Vivo Mucin-type Glycosylation in Escherichia coli,” Microb. Cell Fact 17:168 (2018); Du et al., “A Bacterial Expression Platform for Production of Therapeutic Proteins Containing Human-like O-Linked Glycans,” Cell Chem. Biol. 26:203-212 e205 (2019); and Valentine et al., “Immunization with Outer Membrane Vesicles Displaying Designer Glycotopes Yields Class-switched, Glycan-specific Antibodies,” Cell Chem. Biol. 23:655-665 (2016), which are hereby incorporated by reference in their entirety). To address the lack of known enzymes that form Und-PP-GalNAc in E. coli, PglC from Acinetobacter baumannii ATCC 17978 (AbPglC), which specifically transfers GalNAc to Und-PP in A. baumannii cells (Harding et al., “Distinct Amino Acid Residues Confer One of Three UDP-sugar Substrate Specificities in Acinetobacter baumannii PglC Phosphoglycosyltransferases,” Glycobiology 28:522-533 (2018), which is hereby incorporated by reference in its entirety), was enlisted. Together, the CjGne and AbPglC enzymes comprised a putative pathway for Tn antigen biosynthesis.

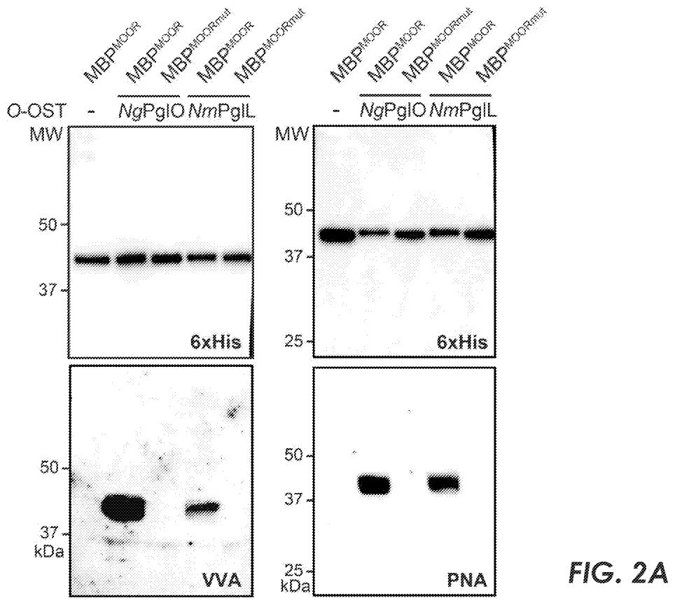

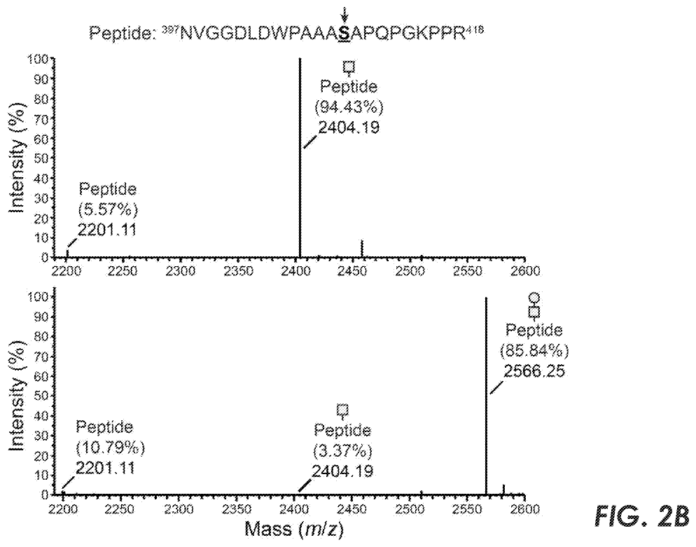

To transfer Und-PP-linked Tn antigen to hydroxylated amino acids in target proteins, the focus was on the bacterial O-OST NmPglL and its ortholog NgPglO (95% identity). It was hypothesized that these enzymes would recognize preassembled O-glycans on Und-PP and transfer them en bloc to Sec-translocated protein substrates in the periplasm (FIG. 1B). The rationale for this hypothesis was based on earlier findings that NmPglL can be functionally expressed in E. coli, leading to transfer of several structurally diverse glycans assembled on Und-PP (Faridmoayer et al., “Extreme Substrate Promiscuity of the Neisseria Oligosaccharyl Transferase Involved in Protein O-glycosylation,”J. Biol. Chem. 283:34596-34604 (2008) and Pan et al., “Biosynthesis of Conjugate Vaccines Using an O-Linked Glycosylation System,” MBio. 7:e00443-00416 (2016), which are hereby incorporated by reference in their entirety). To test this hypothesis, an O-OST gene was added to the Tn pathway yielding plasmids pOG-Tn-NmPglL and pOG-Tn-NgPglO. In parallel, a pEXT20-based plasmid encoding E. coli maltose-binding protein (MBP) modified at its N-terminus with the periplasmic targeting signal derived from E. coli DsbA (Fisher et al., “Production of Secretory and Extracellular N-linked Glycoproteins in Escherichia coli,” Appl. Environ. Microbial. 77:871-881 (2011), which is hereby incorporated by reference in its entirety) and at its C-terminus with a MOOR (minimum optimal O-linked recognition) motif that was previously optimized for recognition by NmPglL (Pan et al., “Biosynthesis of Conjugate Vaccines Using an O-Linked Glycosylation System,” MBio. 7:e00443-00416 (2016), which is hereby incorporated by reference in its entirety) was created. CLM25 cells co-transformed with these two plasmids produced MBP^{MOOR} that was strongly glycosylated with the Tn antigen as revealed by immunoblots probed with Vicia villosa agglutinin (VVA), a lectin that preferentially binds single αGalNAc residues linked to serine or threonine (FIG. 2A). Importantly, glycosylation was completely undetectable when either O-OST was absent or the serine residue in the MOOR tag was substituted with glycine (MOOR^{mut}).

The glycosylated MBP^{MOOR} was further examined by nanoscale liquid chromatography coupled to tandem mass spectrometry (nano-LC-MS/MS) to identify the modification sites.

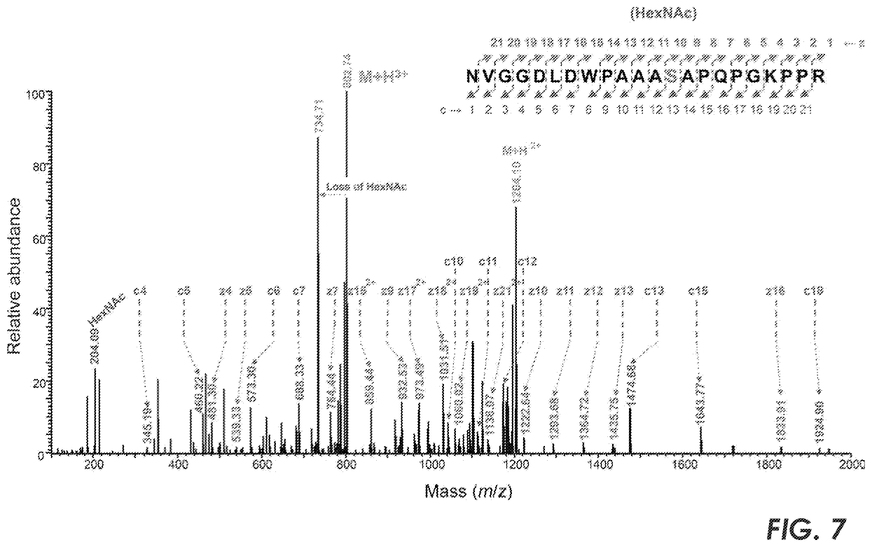

Glycosylation with only HexNAc was identified as the predominant species while a much smaller amount of aglycosylated peptide was also detected (FIG. 2B), consistent with immunoblot analysis. Electron-transfer/higher-energy collision dissociation (EThcD) fragmentation analysis was subsequently performed and unambiguously identified HexNAc modification on S409 within the MOOR sequence of MBP^{MOOR} (FIG. 7). Taken together, these results unequivocally established a route for orthogonal biosynthesis of Tn-modified O-glycoproteins.

### Example 2—Pathway Extension Enables T Antigen Biosynthesis

Next, biosynthesis of the T antigen (Gal-β1,3-GalNAcα), another mucin-type O-glycan that is absent in most normal tissues but present in many human cancers (Tarp M A & Clausen H, “Mucin-type O-glycosylation and its Potential Use in Drug and Vaccine Development,” Biochim. Biophys. Acta. 1780:546-563 (2008), which is hereby incorporated by reference in its entirety), was attempted. The challenge here was the fact that Und-PP-GalNAc represents an atypical substrate for eukaryotic Gal transferases (GalT) that prefer GalNAcα-O-S/T. Therefore, a panel of GalT enzymes were evaluated. The panel included: core 1 synthase glycoprotein-N-acetylgalactosamine 3-β-galactosyltransferase from Homo sapiens (HsClGalT1) and Drosophila melanogaster (DmClGalT1); Bifidobacterium infantis D-galactosyl-β1-3-N-acetyl-D-hexosamine phosphorylase (BiGalHexNAcP); the “S42” mutant of C. jejuni β1-3-galactosyltransferase (CjCgtB) engineered with improved catalytic activity (Yang et al., “Fluorescence Activated Cell Sorting as a General Ultra-high-throughput Screening Method for Directed Evolution of Glycosyltransferases,” J. Am. Chem. Soc. 132:10570-10577 (2010), which is hereby incorporated by reference in its entirety); and β-1,3-galactosyltransferases from enteropathogenic E. coli O86 (EcWbnJ) and enterohemorrhagic E. coli O104 (EcWbwC).

To screen GalT activity, a high-throughput flow cytometric assay was adapted (Valderrama-Rincon, “An Engineered Eukaryotic Protein Glycosylation Pathway in Escherichia coli.,” Nat. Chem. Biol. 8:434-436 (2012) and Glasscock et al., “A Flow Cytometric Approach to Engineering Escherichia coli for Improved Eukaryotic Protein Glycosylation,” Metab. Eng. 47:488-495 (2018), which are hereby incorporated by reference in their entirety). In this assay, Und-PP-linked glycans are flipped into the periplasm by the native E. coli flippase, Wzx, and transferred onto lipid A-core by the O-antigen ligase, WaaL (FIG. 8A). Upon shuttling to the outer membrane, lipid A-core displays the attached glycan on the cell surface, where it is readily detected by fluorescently tagged antibodies or lectins. When screened by flow cytometry using FITC-conjugated Arachis hypogaea peanut agglutinin (PNA) lectin, which recognizes T antigen, only cells expressing EcWbwC were observed to transfer galactose to Und-PP-linked GalNAc (FIG. 8B); hence, co-expression of CjGne, AbPglC, and EcWbwC from plasmid pOG-T was used for all experiments involving T antigen or derivatives thereof. Importantly, EcWbwC activity was dependent on CjGne, which converts UDP-GlcNAc to UDP-GalNAc (FIG. 8C), confirming that the reducing-end monosaccharide was indeed GalNAc.

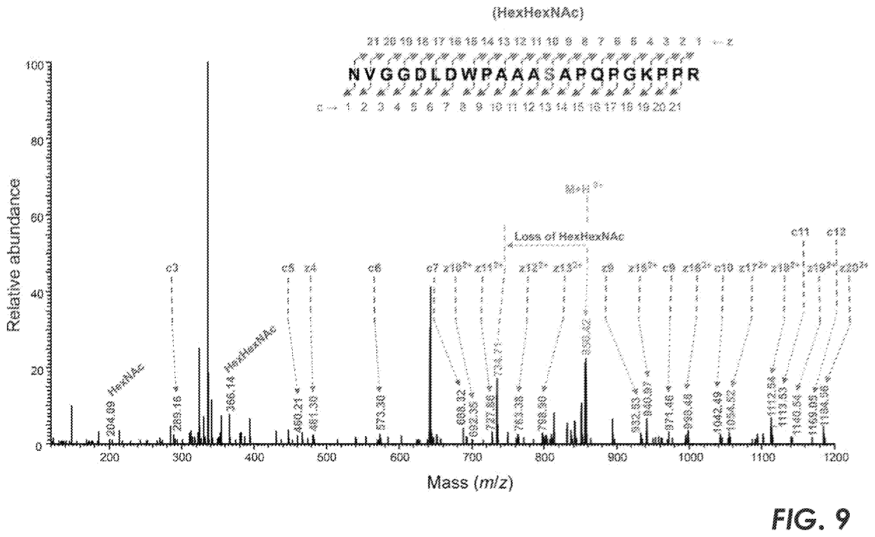

To transfer T antigen to proteins, O-OST genes were added to the T antigen pathway, yielding plasmids pOG-T-NmPglL and pOG-T-NgPglO. CLM25 cells co-transformed with one of these plasmids along with the plasmid encoding MBP^{MOOR} produced acceptor proteins that were glycosylated with T antigen as revealed by immunoblots probed with PNA (FIG. 2A). As expected, this glycosylation depended on the O-OST and the serine residue in the MOOR tag. Nano-LC-MS/MS analysis revealed glycosylation with HexHexNAc as the predominant species (FIG. 2B), indicating efficient T antigen assembly and transfer to protein by orthogonal pathway enzymes. EThcD fragmentation analysis again confirmed HexHexNAc modification on S409 of MBP^{MOOR} (FIG. 9).

### Example 3—Orthogonal Biosynthesis of Sialylated O-Glycoforms

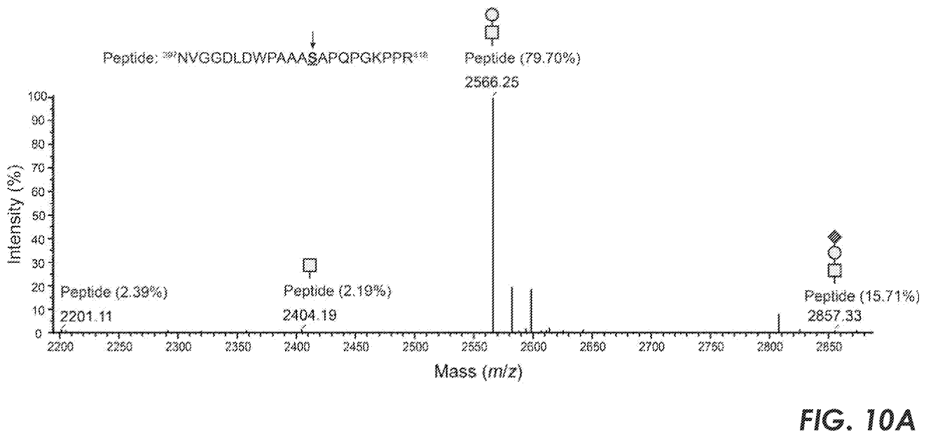

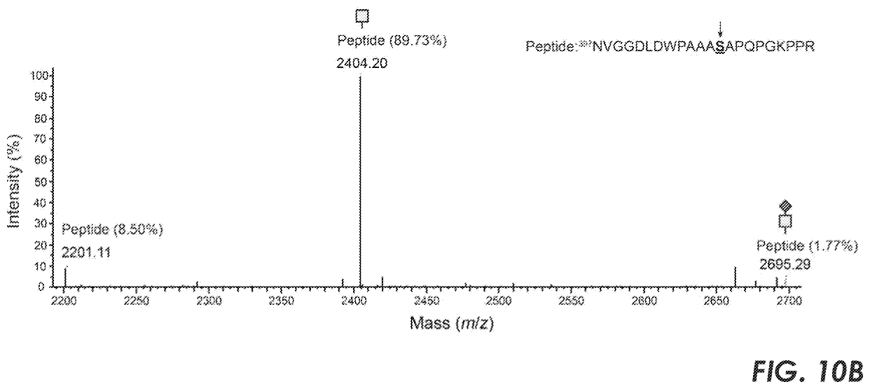

To produce O-glycans bearing sialic acid (NeuNAc), including the STn (NeuNAc-α2,6-GalNAcα) and ST antigens (NeuNAc-α2,3-Gal-β 1,3-GalNAcα) (FIG. 1A) that are commonly observed in cancer, required engineering of our host strain to generate CMP-NeuNAc. To this end, a plasmid encoding the E. coli K1 neuDBAC genes was constructed (FIG. 3A), which enable production of CMP-NeuNAc from UDP-GlcNAc in K-12 strains (Valentine et al., “Immunization with Outer Membrane Vesicles Displaying Designer Glycotopes Yields Class-switched, Glycan-specific Antibodies,” Cell. Chem. Biol. 23:655-665 (2016), which is hereby incorporated by reference in its entirety). In addition, the nanA gene encoding N-acetylneuraminate lyase was deleted from the genome of our host strain to avoid catabolism of CMP-NeuNAc. LC-MS analysis confirmed that nanA-deficient cells carrying the CMP-NeuNAc pathway plasmid produced significant levels of CMP-NeuNAc (FIG. 3B). Next, the gene encoding E. coli O104 WbwA (EcWbwA) sialyltransferase, which the inventors predicted would modify Und-PP-linked T antigen with α2,3-linked NeuNAc, was added to the MBP^{MOOR} expression plasmid. When this latter plasmid was added to nanA-deficient cells carrying the CMP-NeuNAc pathway and pOG-T-NgPglO plasmids, glycosylation of MBP^{MOOR} with NeuNAcHexHexNAc was observed (FIG. 10A). However, the HexHexNAc-modified glycoform was significantly more abundant, suggesting inefficient extension of T antigens with NeuNAc in this host.

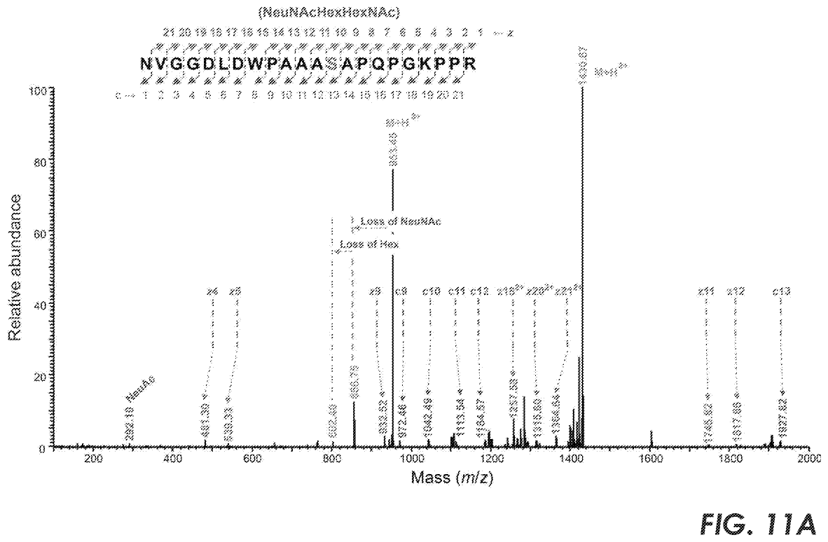

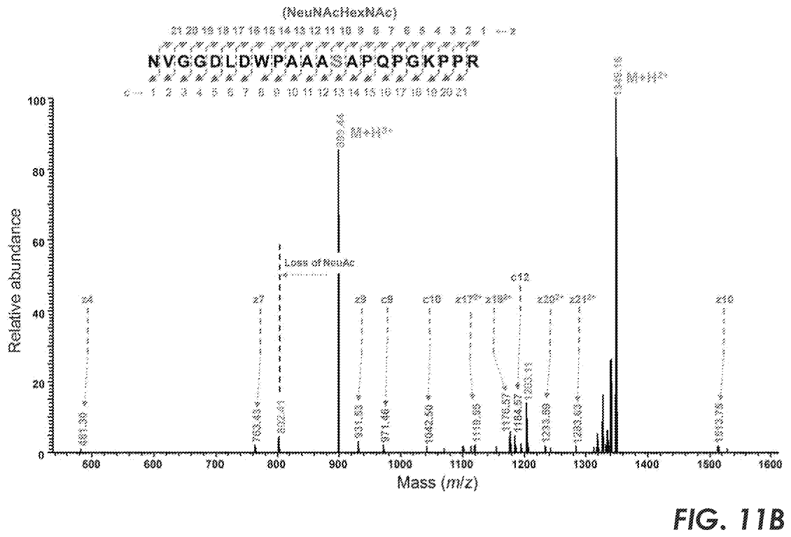

It as speculated that this low efficiency might be overcome by chromosomal integration of the multi-gene CMP-NeuNAc pathway, a strategy that previously increased glycosylation efficiency of an orthogonal N-linked pathway (Yates et al., “Glyco-recoded Escherichia coli: Recombineering-based Genome Editing of Native Polysaccharide Biosynthesis Gene Clusters,” Metab. Eng. 53:59-68 (2019), which is hereby incorporated by reference in its entirety). To test this notion, a glyco-recoding strategy (Yates et al., “Glyco-recoded Escherichia coli: Recombineering-based Genome Editing of Native Polysaccharide Biosynthesis Gene Clusters,” Metab. Eng. 53:59-68 (2019), which is hereby incorporated by reference in its entirety) was used to integrate the CMP-NeuNAc pathway in place of the non-essential O-polysaccharide (O-PS) antigen biosynthesis pathway in the genome (FIG. 3A). The net effect was a reduction in both the number of required plasmids and the copy number of the neu genes. Following genomic replacement of the O-PS pathway with the CMP-NeuNAc pathway in nanA-deficient cells, appreciable intracellular accumulation of CMP-NeuNAc was again observed (FIG. 3B). While the overall CMP-NeuNAc concentration was lower compared to the plasmid-based system, the amount of sialylated glycan on MBP^{MOOR} was dramatically increased in the glyco-recoded host strain, with this glycan representing the most abundant glycoform (FIG. 3C) and occurring on the expected S409 glycosite (FIG. 11A).

A nearly identical strategy for producing STn antigen was carried out using the same glyco-recoded host strain carrying plasmid pOG-Tn-NgPglO in place of pOG-T-NgPglO and the pEXT-based acceptor protein plasmid with α2,6-sialyltransferase from Photobacterium sp. JT-ISH-224 in place of EcWbwA. These cells generated MBP^{MOOR} bearing STn antigen albeit with relatively low sialylation (FIG. 10B; FIG. 11B). Nonetheless, these results showcase the modularity of the O-glycosylation platform, with the introduction of appropriate GTs providing a direct route to more elaborated glycan structures.

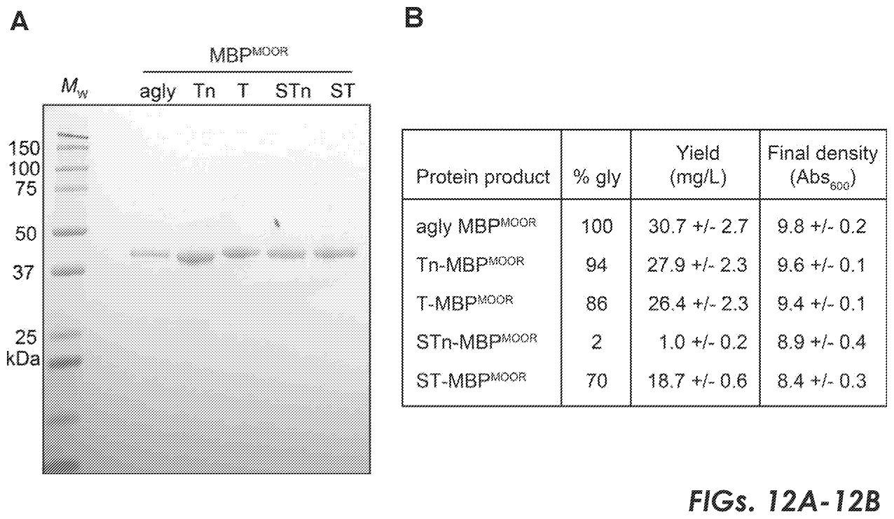

On average, ˜30 mg/L of glycosylated MBP^{MOOR} with each of the different O-glycan structures was produced from small-scale cultures (FIGS. 12A-12B). These yields compared favorably to the yields of 60-80 mg/L obtained previously for processive glycosylation of target proteins with T antigen in the E. coli cytoplasm (Du et al., “A Bacterial Expression Platform for Production of Therapeutic Proteins Containing Human-like O-Linked Glycans,” Cell. Chem. Biol. 26:203-212 e205 (2019), which is hereby incorporated by reference in its entirety). It should also be noted that final culture densities of all glycoprotein-producing strains were comparable to that of the control strain expressing aglycosylated MBP^{MOOR} (FIG. 12B).

### Example 4—Cell-Free Extracts Catalyze O-Glycosylation

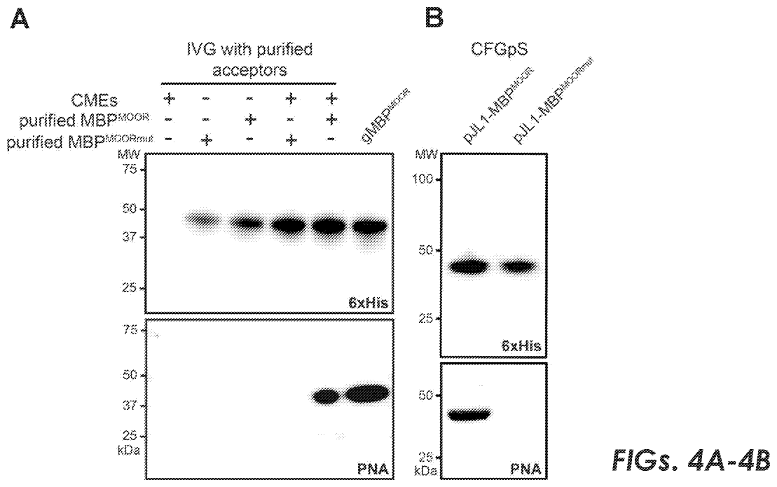

Cell-free modalities are emerging as useful glycoscience tools and for on-demand biomanufacturing of glycoprotein products (Jaroentomeechai et al., “Single-Pot Glycoprotein Biosynthesis Using a Cell-Free Transcription-Translation System Enriched with Glycosylation Machinery,” Nat. Commun. 9:2686 (2018) and Kightlinger et al., “A Cell-Free Biosynthesis Platform for Modular Construction of Protein Glycosylation Pathways,” Nat. Commun. 10:5404 (2019), which are hereby incorporated by reference in their entirety). However, there are currently no cell-free platforms for total biosynthesis of O-glycoproteins. To address this gap, an in vitro glycosylation strategy that combined purified acceptor proteins with partially purified glycosylation machinery was first evaluated. Crude membrane extracts selectively enriched with NgPglO and UndPP-linked T antigen were prepared from CLM25 cells carrying plasmid pOG-T NgPglO. Upon addition of purified acceptor protein to these “glyco-enriched” extracts, clear glycosylation was observed (FIG. 4A). Next, a more integrated approach in which cell-free transcription, translation, and glycosylation were carried out together in a single pot was attempted. This involved preparing crude S12 extracts from the same CLM25 cells carrying plasmid pOG-T NgPglO. To initiate cell-free glycoprotein synthesis (CFGpS), the resulting glyco-enriched S12 extracts containing Und-PP-linked T antigen and NgPglO were primed with plasmid DNA encoding the acceptor protein. Following this reaction, clearly detectable MBP^{MOOR} glycosylation was observed, whereas no glycosylation was detected in reactions charged with plasmid DNA encoding MBP^{MOOR} (FIG. 4B). These results establish that orthogonal O-glycosylation can be functionally reconstituted outside the cell, giving rise to one-pot O-glycoprotein biosynthesis.

### Example 5-O-Glycosylation of Diverse Acceptor Protein Targets

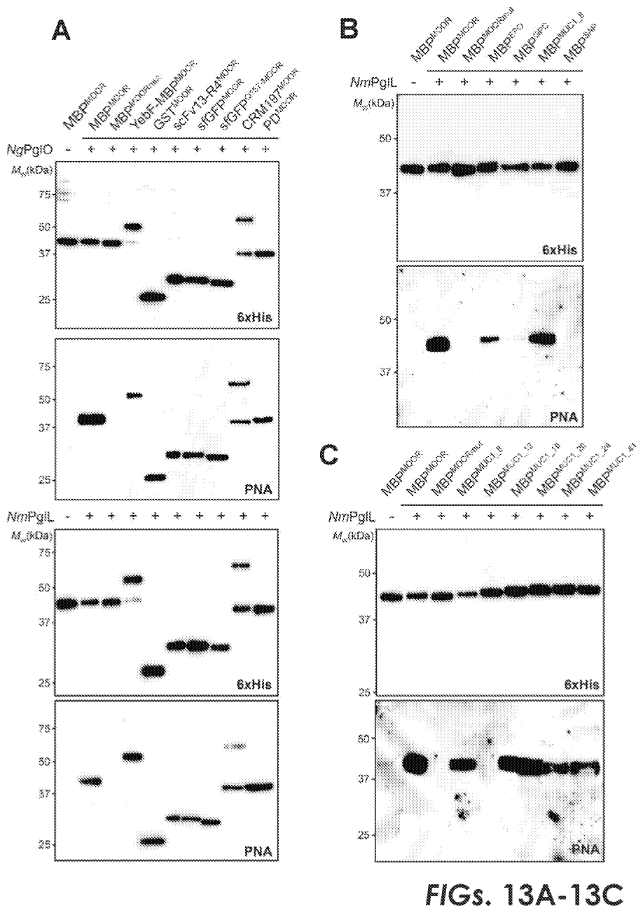

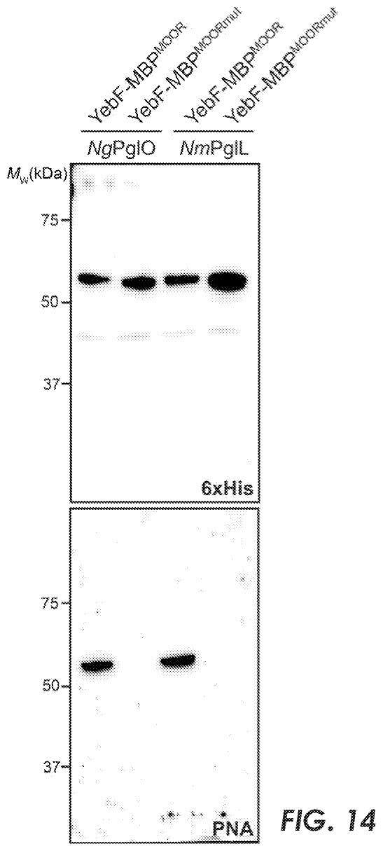

To determine the range of glycosylatable acceptor proteins, the MOOR tag was grafted onto the C-terminus of several proteins including: E. coli glutathione-S-transferase (GST); a single-chain Fv antibody fragment specific for β-galactosidase (scFv13-R4); and two conjugate vaccine carrier proteins, namely cross-reacting material 197 (CRM197) and Haemophilus influenzae protein D (PD). A chimera comprised of E. coli secretory protein YebF fused to MBP^{MOOR} as well as two variants of superfolder GFP (sfGFP), one with a C-terminal MOOR tag and the other with the MOOR motif grafted in an internal loop starting at Gln157 were also created. It should be noted that scFv13-R4, sfGFP, and YebF have all been N-glycosylated in E. coli previously (Valderrama-Rincon, “An Engineered Eukaryotic Protein Glycosylation Pathway in Escherichia coli.,” Nat Chem. Biol. 8:434-436 (2012); Jaroentomeechai et al., “Single-pot Glycoprotein Biosynthesis Using a Cell-free Transcription-translation System Enriched with Glycosylation Machinery,” Nat. Commun. 9:2686 (2018); Ollis et al., “Engineered Oligosaccharyltransferases with Greatly Relaxed Acceptor-site Specificity,” Nat. Chem. Biol. 10:816-822 (2014); and Fisher et al., “Production of Secretory and Extracellular N-linked Glycoproteins in Escherichia coli,” App. Environ. Microbiol. 77:871-881 (2011), which are here by incorporated by reference in their entirety) while CRM 197 and PD represent carrier proteins used in licensed conjugate vaccines. When expressed in the presence of the T antigen pathway, each protein cross-reacted with PNA (FIG. 13A), confirming that O-glycosylation was compatible with different protein contexts including terminal and internal locations. It is also noteworthy that YebF-MBP^{MOOR} and YebF-MBP^{MOORmut} both accumulated in the extracellular culture medium with only YebF-MBP^{MOOR} cross-reacting with PNA (FIG. 14), indicating that YebF-mediated secretion is harmonious with en bloc O-glycosylation, as it was for N-glycosylation (Fisher et al., “Production of Secretory and Extracellular N-linked Glycoproteins in Escherichia coli,” Appl. Environ. Microbiol. 77:871-881 (2011), which is hereby incorporated by reference in its entirety).

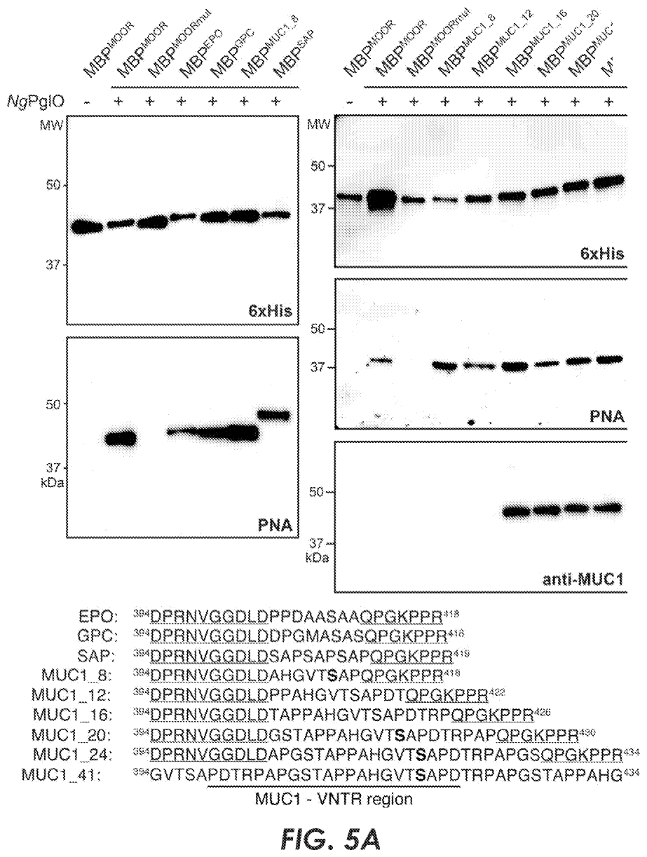

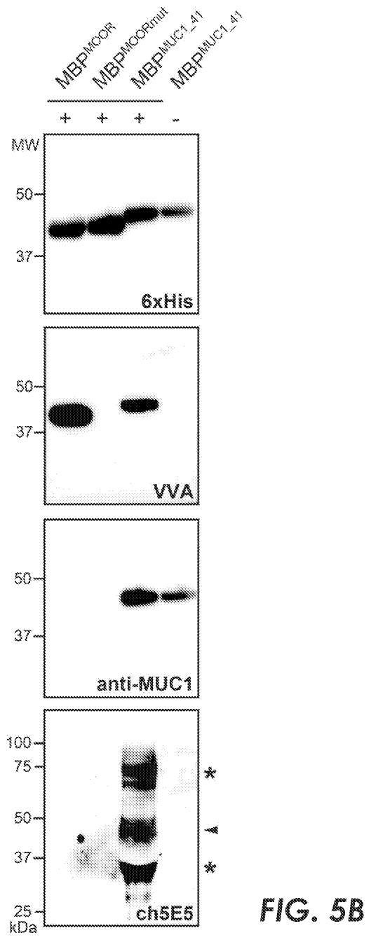

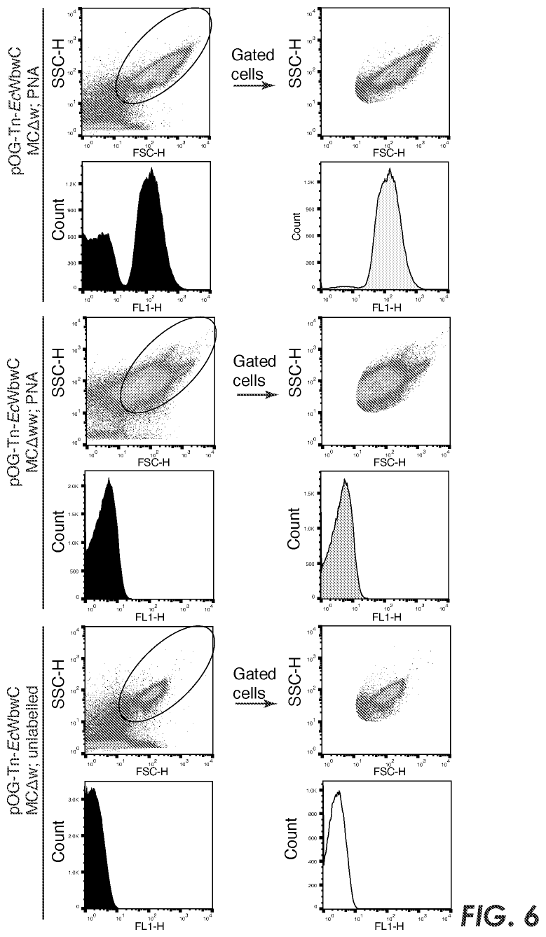

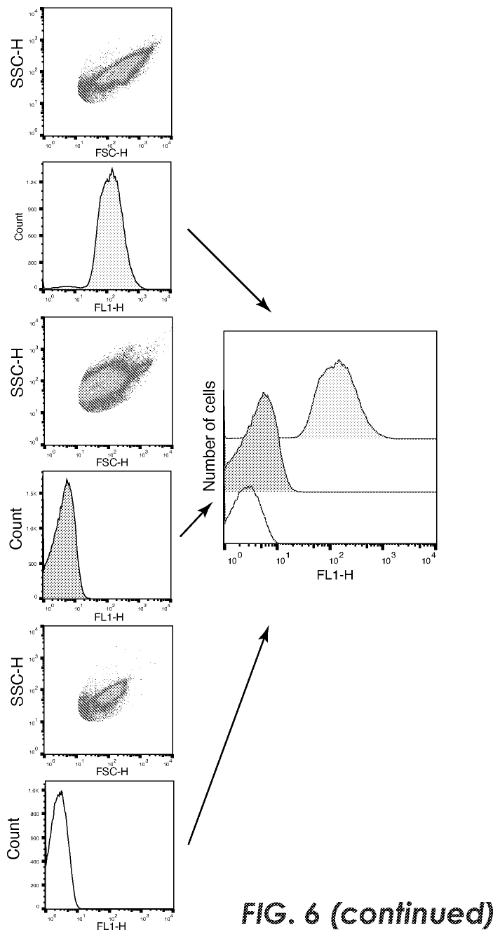

System modularity was further evaluated by swapping the 8-residue core sequence of the MOOR tag with different human or synthetic O-glycosylation motifs. These included: 8 residues surrounding the S126 O-glycosite in human erythropoietin (EPO) (Lai et al., “Structural Characterization of Human Erythropoietin,” J. Biol. Chem. 261:3116-3121 (1986), which is hereby incorporated by reference in its entirety); 8 residues surrounding the S24 O-glycosite in human glycophorin C (GPC), a surface glycoprotein found on red blood cells that marks the Gerbich antigen system (Maier et al., “Plasmodium Falciparum Erythrocyte Invasion Through Glycophorin C and Selection for Gerbich Negativity in Human Populations,” Nat. Med. 9:87-92 (2003), which is hereby incorporated by reference in its entirety); 8 residues derived from the ectodomain of human mucin 1 (MUC1), which is expressed on the apical surface of glandular epithelial cells at low levels but following oncogenic transformation is expressed at very high levels and with altered glycosylation (Tarp M A & Clausen H, “Mucin-type O-glycosylation and its Potential Use in Drug and Vaccine Development,” Biochim. Biophys. Acta 1780:546-563 (2008), which is hereby incorporated by reference in its entirety); and synthetic “SAP” motif that was designed de novo based on known glycosite preferences of NmPglL (Pan et al., “Biosynthesis of Conjugate Vaccines Using an O-Linked Glycosylation System,” MBio. 7:e00443-00416 (2016), which is hereby incorporated by reference in its entirety). When each construct was expressed in the presence of NgPglO, strong glycosylation with T antigen was observed (FIG. 5A). Interestingly, while NmPglL also robustly glycosylated the EPO- and MUC1-derived sequences, it showed weak glycosylation of the GPC derived sequence and no detectable activity towards the SAP sequence (FIG. 13B), revealing subtle differences in O-OST substrate selectivity. Collectively, these results highlight the ability of our platform to modify O-glycosites in human proteins.

### Example 6—Biosynthesis of Antigenically-Relevant MUC1 Glycoforms

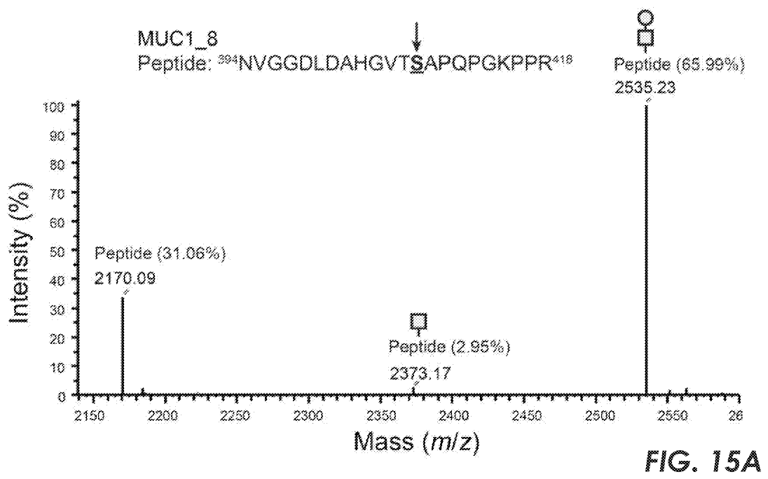

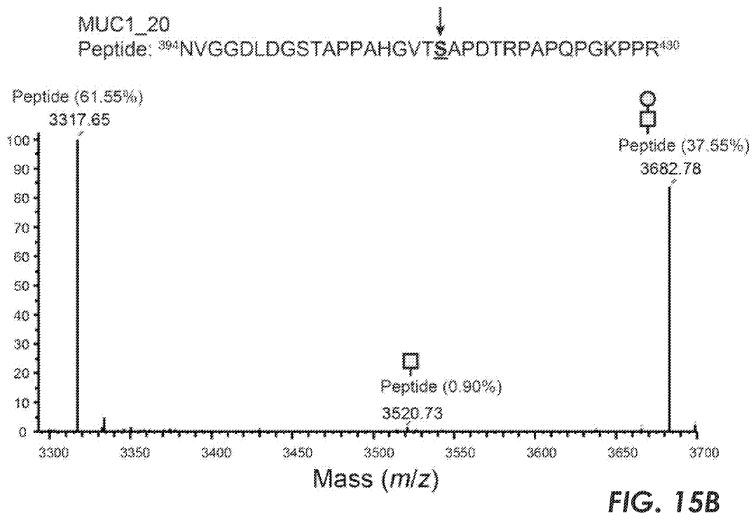

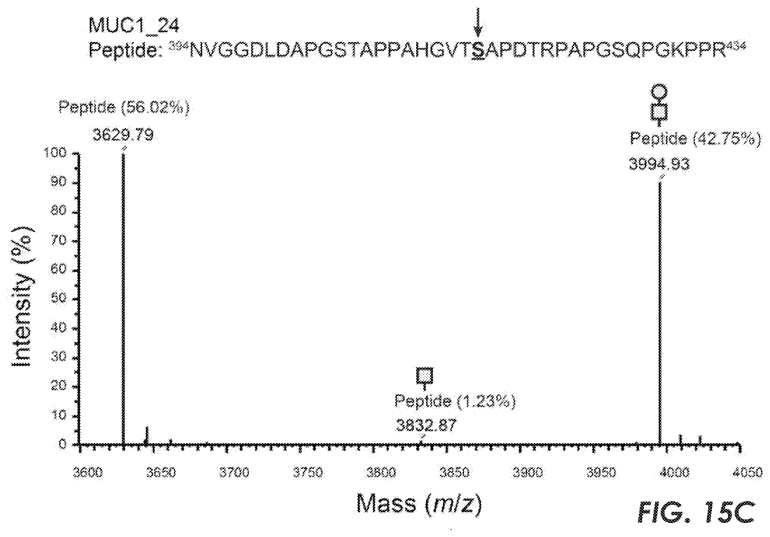

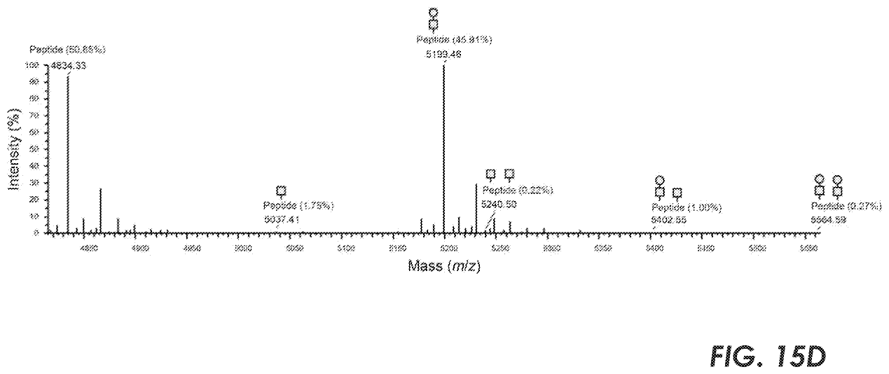

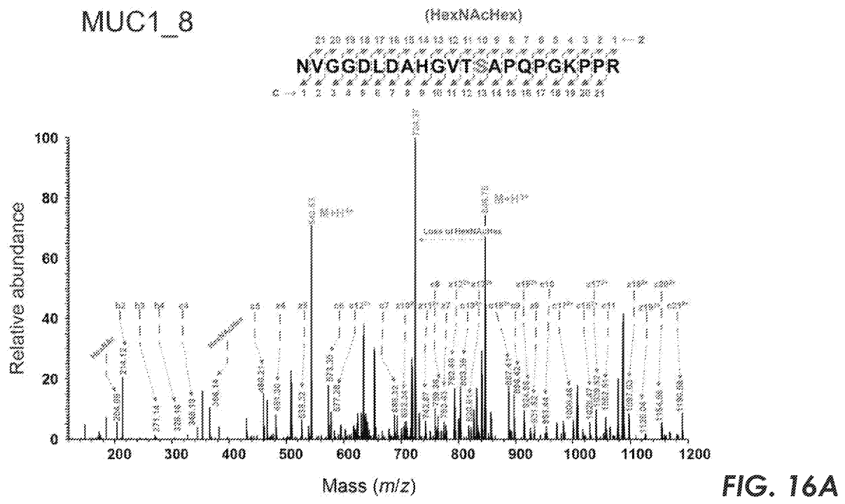

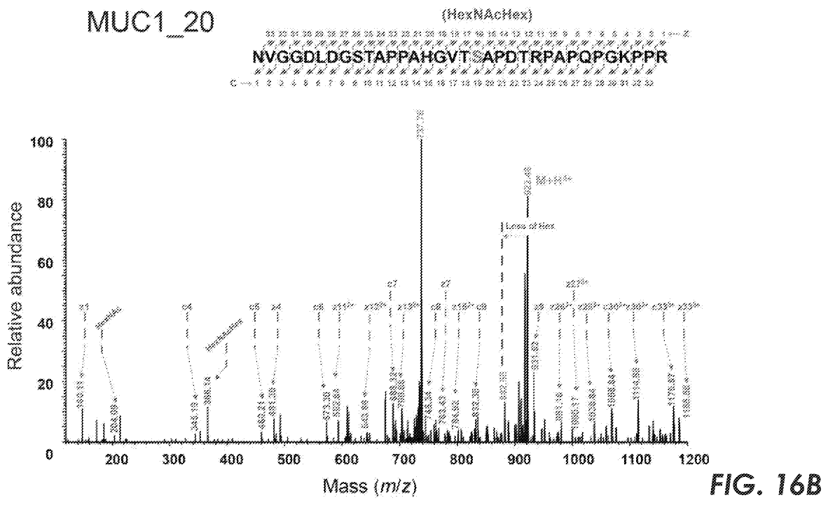

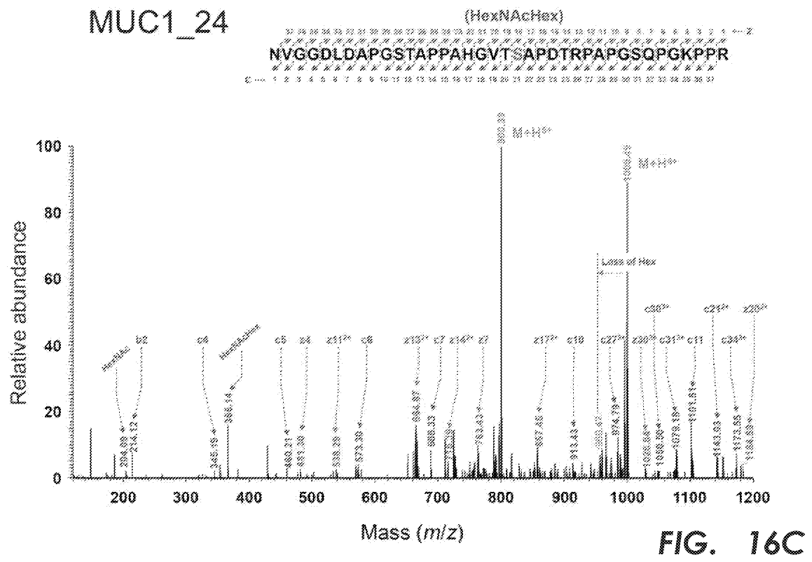

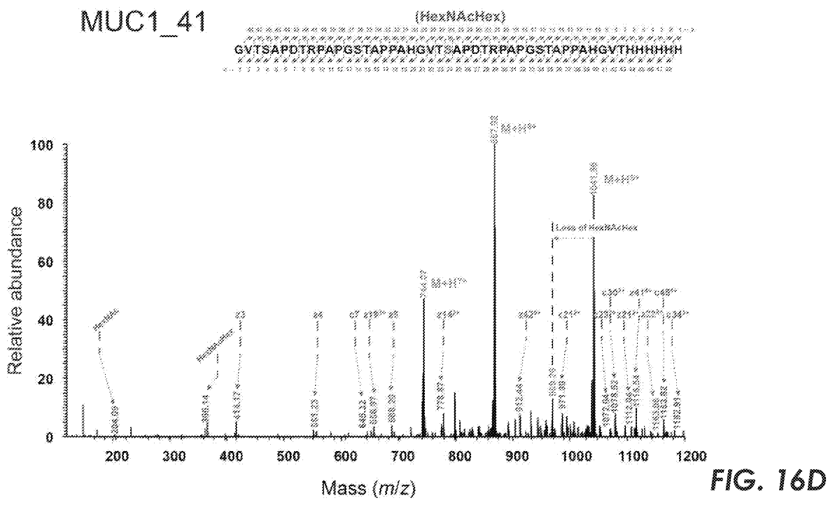

To generate additional MUC1 glycoforms with relevance to human cancer, the variable number of tandem repeats (VNTRs) of MUC1 that consist of 20-120 repeats of a 20-amino acid sequence (PDTRPAPGSTAPPAHGVTSA (SEQ ID NO: 36)) and contain five potential O-glycosylation sites (bold) was the next focus of these studies (Gendler et al., “A Highly Immunogenic Region of a Human Polymorphic Epithelial Mucin Expressed by Carcinomas is Made Up of Tandem Repeats;” J. Biol. Chem. 263:12820-12823 (1988), which is hereby incorporated by reference in its entirety). Here, four VNTR-derived sequences were created by incrementally extending the MUC1_8 motif. Each of these was cloned between the hydrophilic flanking regions of the MOOR motif and subsequently expressed in CLM25 cells carrying either pOG-T-NgPglO or pOG-T-NmPglL. The T antigen-producing host strain was chosen because tumor-associated MUC1 is aberrantly glycosylated with truncated O-glycans including T antigen (Tarp M A & Clausen H, “Mucin-type O-glycosylation and its Potential Use in Drug and Vaccine Development,” Biochim. Biophys. Acta 1780:546-563 (2008), which is hereby incorporated by reference in its entirety). Following expression in bacteria carrying the T antigen pathway, each MUC1 motif was strongly glycosylated by NgPglO (FIG. 5A). NmPglL similarly modified all these motifs except for MUC1_12, which was not detectably glycosylated (FIG. 13C) and indicated another subtle difference in O-OST substrate selectivity. It should also be noted that MUC1_16, MUC1_20, and MUC1_24 each cross-reacted with the mouse monoclonal antibody H23 (FIG. 5A), which recognizes the MUC1 APDTRP epitope on the surface of human breast cancer cells (Mazur et al., “Humanization and Epitope Mapping of the H23 anti-MUC1 Monoclonal Antibody Reveals a Dual Epitope Specificity,” Mol. Immunol. 42:55-69 (2005), which is hereby incorporated by reference in its entirety) and confirms the antigenic relevance of these MUC1 peptides. HexHexNAc-modified MUC1_8, MUC1_20, and MUC1_24 were identified as the predominant glycoforms (FIGS. 15A-15C), with the most abundant glycoforms corresponding to HexHexNAc modification at the same serine residue in each construct (FIGS. 16A-16D).

To generate more antigenically authentic glycoforms, a 41-residue MUC1 sequence containing the 20-residue VNTR flanked with additional stretches of the MUC1 repeat but without the original MOOR flanking residues was investigated. Importantly, both NgPglO and NmPglL were able to transfer T antigen to this construct (FIG. 5B; FIG. 13C). A single HexHexNAc modification on MUC_41 was the predominant glycoform and was found on the same serine residue identified above (FIGS. 16A-16D). In addition to aglycosylated peptide, other minor T and Tn modifications were also detected (FIG. 15D), suggesting multiply glycosylated forms. Targeted HCD and ETD MS/MS analysis was attempted to identify and map the location of these minor glycan modifications; however, glycosites were not able to be assigned because of the lower intensities of these glycopeptides and the lack of key fragments on the MS/MS spectrum needed for unambiguous site assignment. Low-resolution ion trap-based detection of ETHcD fragments was also unable to yield conclusive evidence for additional O-glycosylation beyond the S417 modification. Nonetheless, these results demonstrate that authentic human O-glycoprotein epitopes can be generated using the engineered glycosylation system described herein without the need for hydrophilic flanking regions.

As was seen for the other APDTRP-containing MUC1 sequences, T-modified MUC1_41 cross-reacted with H23 (FIG. 5B). While this result confirmed creation of an antigenically-intact MUC1 epitope, H23 binding was not dependent on the O-glycan, consistent with the known specificity of this antibody (Mazor et al., “Humanization and Epitope Mapping of the H23 anti-MUC1 Monoclonal Antibody Reveals a Dual Epitope Specificity,” Mol. Immunol. 42:55-69 (2005), which is hereby incorporated by reference in its entirety). In contrast, the murine monoclonal antibody 5E5 binds all Tn and STn glycoforms of the MUC1 tandem repeat but does not bind aglycosylated MUC1 peptides (Sorensen et al., “Chemoenzymatically Synthesized Multimeric Tn/STn MUC1 Glycopeptides Elicit Cancer-specific Anti-MUC1 Antibody Responses and Override Tolerance,” Glycobiology 16:96-107 (2006), which is hereby incorporated by reference in its entirety). To determine whether MUC1 glycoforms could be produced that cross-reacted with this glycoform-specific antibody, the MUC1_41 construct was expressed in the presence of the Tn pathway, yielding strongly glycosylated MUC1_41 (FIG. 5B). Importantly, the Tn-modified MUC1_41 but not its aglycosylated counterpart was readily detected by the glycoform-specific antibody. This same antibody did not show reactivity for MBP^{MOOR} bearing Tn antigen, consistent with the fact that both glycan and underlying peptide are required for recognition (Sorensen et al., “Chemoenzymatically Synthesized Multimeric Tn/STn MUC1 Glycopeptides Elicit Cancer-specific Anti-MUC1 Antibody Responses and Override Tolerance,” Glycobiology 16:96-107 (2006), which is hereby incorporated by reference in its entirety). Overall, this glycoform-dependent reactivity provides important validation of the glycoengineered bacteria described herein as a platform for producing glycoprotein epitopes that are antigenically distinct and relevant to cancer immunotherapy.

### Discussion of Examples 1-6

In this work, orthogonal O-glycoprotein biosynthesis in E. coli was engineered by rewiring the cell's metabolism to provide necessary sugar donors and ectopically expressing specific GTs and OSTs from diverse organisms. The system was highly modular as evidenced by the ability to generate multiple O-glycan structures and post-translationally modify a panel of acceptor protein targets. Unlike previous mucin-type O-glycoengineering in E. coli that focused on processive glycosylation mechanisms (Henderson et al., “Site-specific Modification of Recombinant Proteins: A Novel Platform for Modifying Glycoproteins Expressed in E. coli,” Bioconjug. Chem. 22:903-912 (2011); Mueller et al., “High Level In Vivo Mucin-Type Glycosylation in Escherichia coli,” Microb. Cell Fact. 17:168 (2018); and Du et al., “A Bacterial Expression Platform for Production of Therapeutic Proteins Containing Human-like O-Linked Glycans,” Cell Chem. Biol. 26:203-212 e205 (2019), which are hereby incorporated by reference in their entirety), an unconventional approach based on the en bloc O-glycosylation mechanism found natively in some bacteria was investigated. Although modeled after this process, the collection of synthetic O-glycosylation pathways described herein has no direct biological equivalent and includes the first biosynthetic routes to sialylated mucin-type O-glycosylation in E. coli.

One advantage of the strategy described herein is the opportunity to leverage diverse enzymes from all domains of life that naturally operate on lipids as well as proteins. A number of bacteria employ glycomimicry strategies in which endogenous GTs construct human-like oligosaccharides that serve to cloak cell-surface components as a means to evade host immune responses. By enlisting these bacterial GTs, one could further expand the repertoire of O-glycans that can be assembled in E. coli. Moreover, because many human GTs are difficult to functionally express in bacteria, often requiring specialized chaperones or solubility-enhancing fusion partners (Ju T & Cummings R D, “A Unique Molecular Chaperone Cosmc Required for Activity of the Mammalian Core 1 Beta 3-galactosyltransferase,” Proc. Natl. Acad Sci. USA 99:16613-16618 (2002) and Skretas et al., “Expression of Active Human Sialyltransferase ST6GalNAcl in Escherichia coli,” Microb. Cell Fact 8:50 (2009), which are hereby incorporated by reference in their entirety), GTs of microbial origin represent a potential workaround for construction of human-like O-glycans as we demonstrated here.

Another advantage of the strategy described herein is the utilization of bacterial O-OSTs that have an inbuilt ability to transfer glycans onto both serine and threonine residues, whereas human GalNAcT2 used previously is limited to threonine. These enzymes exhibit extreme glycan substrate permissiveness as exemplified by NmPglL (Faridmoayer et al., “Extreme Substrate Promiscuity of the Neisseria Oligosaccharyl Transferase Involved in Protein O-glycosylation,” J. Biol. Chem. 283:34596-34604 (2008) and Pan et al., “Biosynthesis of Conjugate Vaccines Using an O-Linked Glycosylation System,” MBio. 7:e00443-00416 (2016), which are hereby incorporated by reference in their entirety). Here, this promiscuity was leveraged to show that NmPglL and its NgPglO ortholog can transfer human-like O-glycan structures. The compatibility of acceptor sequences with these enzymes is much less understood. While it has been shown that individual O-OSTs can modify multiple protein substrates (Schulz et al., “Identification of Bacterial Protein O-oligosaccharyltransferases and Their Glycoprotein Substrates,” PLoS One 8:e62768 (2013), which is hereby incorporated by reference in its entirety), there is no clear sequon for glycosylation and the O-glycan attachment sites are in flexible, low-complexity regions, thereby hindering glycoprotein engineering efforts. A breakthrough in this regard was the identification of the MOOR motif that together with two additional hydrophilic flanking sequences could be recognized by NmPglL (Pan et al., “Biosynthesis of Conjugate Vaccines Using an O-Linked Glycosylation System,” MBio. 7:e00443-00416 (2016), which is hereby incorporated by reference in its entirety) and, as is shown in the exampled presented herein, NgPglO. Using these hydrophilic flanking sequences, the list of glycosylatable sequences was expanded to include several human and synthetic O-glycosites. The observation that NmPglL and NgPglO could glycosylate varying-length human MUC1 sequences suggested a much greater flexibility than was first reported for these enzymes Pan et al., “Biosynthesis of Conjugate Vaccines Using an O-Linked Glycosylation System,” MBio. 7:e00443-00416 (2016), which is hereby incorporated by reference in its entirety).

Most surprising was the site-directed O-glycosylation of MUC1_41 that lacked the flanking sequences, addressing earlier skepticism about the ability of bacterial O-OSTs to discern mammalian O-glycosites (Du et al., “A Bacterial Expression Platform for Production of Therapeutic Proteins Containing Human-like O-Linked Glycans,” Cell Chem. Biol. 26:203-212 e205 (2019), which is hereby incorporated by reference in its entirety). The O-glycosylated MUC1_41 produced herein was structurally similar to glycopeptides that are reactive towards IgG/IgM antibodies (von Mensdorff-Pouilly et al., “Reactivity of Natural and Induced Human Antibodies to MUC1 Mucin with MUC1 Peptides and n-acetylgalactosamine (GalNAc) Peptides,” Int. J. Cancer 86:702-712 (2000), which is hereby incorporated by reference in its entirety) and human MHC class I molecules (Apostolopoulos et al., “A Glycopeptide in Complex with MHC Class 1 Uses the GalNAc Residue as an Anchor,” Proc. Natl. Acad. Sci. USA 100:15029-15034 (2003), which is hereby incorporated by reference in its entirety). Indeed, recognition of Tn-modified MUC1_41 by a glycoform-specific antibody indicated the creation of an antigenically authentic glycoform. Moreover, the relatively low glycan occupancy on MUC1_41 (˜1 or 2 O-glycans per repeat) may bode well for immunotherapeutic discovery given that a synthetic 60-residue MUC1 tandem-repeat peptide, which was extensively glycosylated (5 O-glycans per repeat), elicited only modest antibody responses (Sorensen et al., “Chemoenzymatically Synthesized Multimeric Tn/STn MUC1 Glycopeptides Elicit Cancer-specific Anti-MUC1 Antibody Responses and Override Tolerance,” Glycobiology 16:96-107 (2006), which is hereby incorporated by reference in its entirety). This weak humoral response results from an inability of antigen-presenting cells to process densely glycosylated MUC1 glycopeptides (Ninkovic T & Hanisch F G, “O-glycosylated Human MUC1 Repeats are Processed In Vitro by Immunoproteasomes,” J. Immunol. 179:2380-2388 (2007), which is hereby incorporated by reference in its entirety). In contrast, a glycopeptide modified with just a single O-glycan elicited more robust antibody titers and also activated cytotoxic T lymphocytes, which amounted to superior tumor prevention (Lakshminarayanan et al., “Immune Recognition of Tumor-associated Mucin MUC1 is Achieved by a Fully Synthetic Aberrantly Glycosylated MUC1 Tripartite Vaccine.” Proc. Natl. Acad. Sci. USA 109:261-266 (2012), which is hereby incorporated by reference in its entirety).

Looking forward, it is anticipated that the platform described herein could find use in the scalable biosynthesis of O-glycoprotein therapeutics and vaccines. To gain access to greater O-glycoprotein structural space may require additional O-OSTs such as those from Bacteroidetes that modify proteins at a minimal 3-residue motif, D-(S/T)-(A/L/V/I/M/T) (Coyne et al., “Phylum-wide General Protein O-glycosylation System of the Bacteroidetes,” Mol. Microbiol. 88:772-783 (2013), which is hereby incorporated by reference in its entirety). Directed evolution of GTs to tailor substrate specificity and metabolic engineering to drive pathway performance towards higher conversion could be enabled through a high-throughput screen for O-glycosylation akin to ‘glycoSNAP’, a bacterial colony blot assay for N-linked glycosylation that was used previously to evolve bacterial N-OST variants with greatly relaxed sequon specificity (Ollis et al., “Engineered Oligosaccharyltransferases with Greatly Relaxed Acceptor-site Specificity,” Nat. Chem. Biol. 10:816-822 (2014), which is hereby incorporated by reference in its entirety). A first important step in this direction was the demonstration that O-glycoproteins can be secreted out of the cell by genetic fusion to the C-terminus of the secretory protein YebF, a feat that is not possible with cytoplasmic O-glycosylation systems. Beyond O-glycoprotein production, the ability of the glycoengineered strains to produce custom glyco-ligands such as O-glycosylated GST and sfGFP could facilitate pulldown assays and cell labeling experiments, respectively, with the potential to uncover and characterize binding partners of structurally defined O-glycoforms. Altogether, the results presented herein define a versatile platform for site-directed O-glycosylation of proteins with different mucin-type O-glycans, thereby expanding the bacterial glycoengineering toolkit.

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. A recombinant prokaryotic host cell expressing one or more 4-epimerases, one or more glycosyl-1-phosphate transferases, and one or more O-oligosaccharyltransferases.

2. A recombinant prokaryotic host cell expressing one or more 4-epimerases, one or more glycosyl-1-phosphate transferases, one or more O-oligosaccharyltransferases, and one or more ß1,3-galactosyltransferase enzymes capable of transferring galactose (Gal) to undecaprenyl pyrophosphate (Und-PP)-linked N-Acetylgalactosamine (GalNAc).

3. The recombinant prokaryotic host cell of claim 1 or 2, wherein the one or more 4-epimerases is a Gne.

4. The recombinant prokaryotic host cell of claim 3, wherein the Gne is C. jejuni Gne (CjGne).

5. The recombinant prokaryotic host cell of claim 1 or 2, wherein the one or more glycosyl-1-phosphate transferases is a PglC.

6. The recombinant prokaryotic host cell of claim 5, wherein the PglC is Acinetobacter baumannii ATCC 17978 PglC (AbPglC).

7. The recombinant prokaryotic host cell of claim 1 or 2, wherein the one or more O-oligosaccharyltransferases is PglL, PglO, or a combination thereof.

8. The recombinant prokaryotic host cell of claim 7, wherein the PglL is a Neisseria meningitides PglL (NmPglL) and the PglO is Neisseria gonorrhoeae PglO (NgPglO).

9. The recombinant prokaryotic host cell of any one of claims 2-9, wherein the one or more ß1,3-galactosyltransferase enzymes capable of transferring galactose (Gal) to undecaprenyl pyrophosphate (Und-PP)-linked N-Acetylgalactosamine (GalNAc) is the ß-1,3; galactosyltransferase derived from the O-antigen biosynthesis pathway of enterohemorrhagic Escherichia coli O104 (EcWbwC).

10. The recombinant prokaryotic host cell of any one of claims 1-9, wherein each of the one or more 4-epimerases, one or more glycosyl-1-phosphate transferases, one or more O-oligosaccharyltransferases, and one or more ß1,3-galactosyltransferase enzymes capable of transferring galactose (Gal) to undecaprenyl pyrophosphate (Und-PP)-linked N-Acetylgalactosamine (GalNAc) are encoded by a polynucleotide sequence that is independently located either on an extrachromosomal plasmid carried by the prokaryotic host cell or in the recombinant prokaryotic host cells genome.

11. The recombinant prokaryotic host cell of any one of claims 1-10, wherein the recombinant prokaryotic host cell does not express an enzymatically active undecaprenyl-phosphate alpha-N-acetylglucosaminyl 1-phosphate transferase.

12. The recombinant prokaryotic host cell of claim 11, wherein the recombinant prokaryotic host cell is an E. coli host cell that lacks a functional copy of the wecA gene.

13. The recombinant prokaryotic host cell of any one of claims 1-12, wherein the recombinant prokaryotic host cell does not express an enzymatically active O-antigen ligase.

14. The recombinant prokaryotic host cell of claim 13, wherein the recombinant prokaryotic host cell is an E. coli host cell that lacks a functional copy of the waaL gene.

15. The recombinant prokaryotic host cell of claim 1, wherein the recombinant prokaryotic host cell further expresses:
(i) the enzymes of an enzymatic pathway capable of producing cytidine-5′-monophosphate-5-N-acetylneuraminic acid (CMP-NeuNAc); and
(ii) one or more α2,6-sialyltransferases.

16. The recombinant prokaryotic host cell of claim 15, wherein the one or more α2,6-sialyltransferases is the α2,6-sialyltransferase from Photobacterium sp. JT-ISH-224.

17. The recombinant prokaryotic host cell of claim 2, wherein the recombinant prokaryotic host cell further expresses:
(i) the enzymes of an enzymatic pathway capable of producing cytidine-5′-monophosphate-5-N-acetylneuraminic acid (CMP-NeuNAc); and
(ii) one or more α2,3-sialyltransferases and/or one or more α2,6-sialyltransferases.

18. The recombinant prokaryotic host cell of claim 17, wherein the one or more α2,3-sialyltransferases is WbwA from E. coli O104 and the one or more α2,6-sialyltransferases is the α2,6-sialyltransferase from Photobacterium sp. JT-ISH-224.

19. The recombinant prokaryotic host cell of any one of claims 15-18, wherein the enzymes of the enzymatic pathway capable of producing cytidine-5′-monophosphate-5-N-acetylneuraminic acid (CMP-NeuNAc) are the E. coli K1 neuDBAC genes.

20. The recombinant prokaryotic host cell of any one of claims 15-19, wherein the recombinant prokaryotic host cell does not express an enzymatically active N-acetylneuraminate lyase.

21. The recombinant prokaryotic host cell of claim 20, wherein the recombinant prokaryotic host cell is an E. coli host cell that lacks a functional copy of the nanA gene.

22. The recombinant prokaryotic host cell of claim any one of claims 15-21, wherein the enzymes of an enzymatic pathway capable of producing cytidine-5′-monophosphate-5-N-acetylneuraminic acid (CMP-NeuNAc) are encoded by a polynucleotide sequence that is located on an extrachromosomal plasmid carried by the prokaryotic host cell.

23. The recombinant prokaryotic host cell of claim any one of claims 15-21, wherein the enzymes of an enzymatic pathway capable of producing cytidine-5′-monophosphate-5-N-acetylneuraminic acid (CMP-NeuNAc) are encoded by a polynucleotide sequence that is located in the recombinant prokaryotic host cell's genome.

24. The recombinant prokaryotic host cell of any one of claims 15-23, wherein each of the one or more α2,3-sialyltransferases or α2,6-sialyltransferases is encoded by a polynucleotide sequence that is independently located either on an extrachromosomal plasmid carried by the prokaryotic host cell or in the recombinant prokaryotic host cell's genome.

25. The recombinant prokaryotic host cell of any one of claims 1-24, wherein the recombinant prokaryotic host cell is an Escherichia coli cell.

26. A method for producing an O-glycosylated protein comprising:
providing a recombinant host cell expressing one more 4-epimerases, one or more glycosyl-1-phosphate transferases, one or more O-oligosaccharyltransferases, and a glycoprotein target comprising one or more serine and/or threonine residues; and
culturing said host cell under conditions effective to:
(i) produce N-acetylgalactosamine (GalNAc) linked to undecaprenyl pyrophosphate (Und-PP); and
(ii) transfer the N-acetylgalactosamine (GalNAc) linked to undecaprenyl pyrophosphate (Und-PP) en bloc to a serine or threonine amino acid of the glycoprotein target.

27. A method for producing an O-glycosylated protein comprising:
providing a recombinant host cell expressing one more 4-epimerases, one or more glycosyl-1-phosphate transferases, one or more O-oligosaccharyltransferases, one or more ß1,3-galactosyltransferase enzymes capable of transferring galactose (Gal) to undecaprenyl pyrophosphate (Und-PP)-linked N-Acetylgalactosamine (GalNAc), and a glycoprotein target comprising one or more serine and/or threonine residues; and
culturing said host cell under conditions effective to:
(i) produce N-acetylgalactosamine (GalNAc) linked to undecaprenyl pyrophosphate (Und-PP);
(ii) extend Und-PP-GalNAc by a single galactose (Gal) monosaccharide to yield lipid-linked Gal-ß1,3-GalNAc; and
(iii) transfer the lipid-linked Gal-ß1,3-GalNAc en bloc to a serine or threonine amino acid of the glycoprotein target.

28. The method of claim 26 or 27, wherein the one or more 4-epimerases is a Gne.

29. The method of claim 28, wherein the Gne is C. jejuni Gne (CjGne).

30. The method of claim 26 or 27, wherein the one or more glycosyl-1-phosphate transferases is a PglC.

31. The method of claim 30, wherein the PglC is Acinetobacter baumannii ATCC 17978 PglC (AbPglC).

32. The method of claim 26 or 27, wherein the one or more O-oligosaccharyltransferases is PglL, PglO, or a combination thereof.

33. The method of claim 32, wherein the PglL is Neisseria meningitides PglL (NmPglL) and the PglO is Neisseria gonorrhoeae PglO (NgPglO).

34. The method of any one of claims 27-33, wherein the one or more ß1,3-galactosyltransferase enzymes capable of transferring galactose (Gal) to undecaprenyl pyrophosphate (Und-PP)-linked N-Acetylgalactosamine (GalNAc) is the ß-1,3,-galactosyltransferase derived from the O-antigen biosynthesis pathway of enterohemorrhagic Escherichia coli O104 (EcWbwC).

35. The method of any one of claims 26-34, wherein each of the one or more 4-epimerases, one or more glycosyl-1-phosphate transferases, one or more O-oligosaccharyltransferases, and one or more ß1,3-galactosyltransferase enzymes capable of transferring galactose (Gal) to undecaprenyl pyrophosphate (Und-PP)-linked N-Acetylgalactosamine (GalNAc) are encoded by a polynucleotide sequence that is independently located either on an extrachromosomal plasmid carried by the prokaryotic host cell or in the recombinant prokaryotic host cell's genome.

36. The method of claim 26, wherein the recombinant prokaryotic host cell further expresses:
(i) the enzymes of an enzymatic pathway capable of producing cytidine-5′-monophosphate-5-N-acetylneuraminic acid (CMP-NeuNAc), and
(ii) one or more α2,6-sialyltransferases; and wherein the N-acetylgalactosamine (GalNAc) linked to undecaprenyl pyrophosphate (Und-PP) is extended by one or more sialic acid (NeuNAc) sugars before the transfer the N-acetylgalactosamine (GalNAc) linked to undecaprenyl pyrophosphate (Und-PP) en bloc to a serine or threonine amino acid of the glycoprotein target.

37. The method of claim 36, wherein the one or more α2,6-sialyltransferases is the α2,6-sialyltransferase from Photobacterium sp. JT-ISH-224.

38. The method of claim 27, wherein the recombinant prokaryotic host cell further expresses:
(i) the enzymes of an enzymatic pathway capable of producing cytidine-5′-monophosphate-5-N-acetylneuraminic acid (CMP-NeuNAc); and
(ii) one or more α2,3-sialyltransferases and/or one or more α2,6-sialyltransferases; and wherein the lipid-linked Gal-ß1,3-GalNAc is extended by one or more sialic acid (NeuNAc) sugars before the transfer the lipid-linked Gal-ß1,3-GalNAc en bloc to a serine or threonine amino acid of the glycoprotein target.

39. The method of claim 38, wherein the one or more α2,3-sialyltransferases is WbwA from E. coli O104 and the one or more α2,6-sialyltransferases is the α2,6-sialyltransferase from Photobacterium sp. JT-ISH-224.

40. The method of any one of claims 36-39, wherein the enzymes of the enzymatic pathway capable of producing cytidine-5′-monophosphate-5-N-acetylneuraminic acid (CMP-NeuNAc) are the E. coli K1 neuDBAC genes.

41. The method of any one of claims 26-40, wherein the recombinant prokaryotic host cell is an Escherichia coli cell.

42. An in vitro method for producing an O-glycosylated protein comprising:
providing glycosylation reagents comprising one more 4-epimerases, one or more glycosyl-1-phosphate transferases, and one or more O-oligosaccharyltransferases,
providing a glycoprotein target comprising one or more serine and/or threonine residues, and
incubating said glycosylation reagents and said glycoprotein target under conditions effective to:
(i) yield N-acetylgalactosamine (GalNAc) linked to undecaprenyl pyrophosphate (Und-PP), and
(ii) transfer the N-acetylgalactosamine (GalNAc) linked to undecaprenyl pyrophosphate (Und-PP) en bloc to a serine or threonine amino acid of the glycoprotein target.

43. An in vitro method for producing an O-glycosylated protein comprising:
providing glycosylation reagents comprising one more 4-epimerase enzymes, one or more heterologous N,N′-diacetylbacilliosaminyl-1-phosphate transferase enzymes, one or more heterologous O-oligosaccharyltransferases, and one or more ß1,3-galactosyltransferase enzymes capable of transferring galactose (Gal) to undecaprenyl pyrophosphate (Und-PP)-linked N-Acetylgalactosamine (GalNAc),
providing a glycoprotein target comprising one or more serine and/or threonine residues, and
incubating said glycosylation reagents and said glycoprotein target under conditions effective to:
(i) yield lipid-linked Gal-ß1,3-GalNAc, and
(ii) transfer the lipid-linked Gal-ß1,3-GalNAc and any additional sugars en bloc to a serine or threonine amino acid of the glycoprotein target.

44. The method of claim 42 or 43, wherein the one or more 4-epimerases is a Gne.

45. The method of claim 44, wherein the Gne is C. jejuni Gne (CjGne).

46. The method of claim 42 or 43, wherein the one or more glycosyl-1-phosphate transferases is a PglC.

47. The method of claim 46, wherein the PglC is Acinetobacter baumannii ATCC 17978 PglC (AbPglC).

48. The method of claim 42 or 43, wherein the one or more O-oligosaccharyltransferases is PglL, PglO, or a combination thereof.

49. The method of claim 48, wherein the PglL is Neisseria meningitides PglL (NmPglL) and the PglO is Neisseria gonorrhoeae PglO (NgPglO).

50. The method of any one of claims 42-49, wherein the one or more ß1,3-galactosyltransferase enzymes capable of transferring galactose (Gal) to undecaprenyl pyrophosphate (Und-PP)-linked N-Acetylgalactosamine (GalNAc) is the ß-1,3,-galactosyltransferase derived from the O-antigen biosynthesis pathway of enterohemorrhagic Escherichia coli O104 (EcWbwC).

51. The method of claim 42, wherein the glycosylation reagents further comprise:
(i) the enzymes of an enzymatic pathway capable of producing cytidine-5′-monophosphate-5-N-acetylneuraminic acid (CMP-NeuNAc); and
(ii) one or more α2,6-sialyltransferases, and wherein the N-acetylgalactosamine (GalNAc) linked to undecaprenyl pyrophosphate (Und-PP) is extended by one or more sialic acid (NeuNAc) sugars before the transfer of the N-acetylgalactosamine (GalNAc) linked to undecaprenyl pyrophosphate (Und-PP) en bloc to a serine or threonine amino acid of the glycoprotein target.

52. The method of claim 51, wherein the one or more α2,6-sialyltransferases is the α2,6-sialyltransferase from Photobacterium sp. JT-ISH-224.

53. The method of claim 43, wherein the glycosylation reagents further comprise:
(i) the enzymes of an enzymatic pathway capable of producing cytidine-5′-monophosphate-5-N-acetylneuraminic acid (CMP-NeuNAc); and
(ii) one or more α2,3-sialyltransferases and/or one or more α2,6-sialyltransferases; and wherein the lipid-linked Gal-ß1,3-GalNAc is extended by one or more sialic acid (NeuNAc) sugars before the transfer the lipid-linked Gal-ß1,3-GalNAc en bloc to a serine or threonine amino acid of the glycoprotein target.

54. The method of claim 53, wherein the one or more α2,3-sialyltransferases is WbwA from E. coli O104 and the one or more α2,6-sialyltransferases is the α2,6-sialyltransferase from Photobacterium sp. JT-ISH-224.

55. The method of any one of claims 51-54, wherein the enzymes of the enzymatic pathway capable of producing cytidine-5′-monophosphate-5-N-acetylneuraminic acid (CMP-NeuNAc) are the E. coli K1 neuDBAC genes.

56. The method of claim 55, wherein the glycosylation reagents are provided in the form of a membrane extract.

57. The method of claim 55, wherein the glycosylation reagents are provided in the form of purified enzymes.

58. An in vitro method for producing an O-glycosylated protein comprising:
providing reagents suitable for synthesizing a glycoprotein target;
providing glycosylation reagents comprising one more 4-epimerases, one or more glycosyl-1-phosphate transferases, and one or more O-oligosaccharyltransferases;
providing a nucleic acid molecule encoding a glycoprotein target; and
incubating said reagents suitable for synthesizing a glycoprotein target, glycosylation reagents, and nucleic acid molecule encoding a glycoprotein target under conditions effective to:
(i) synthesize the glycoprotein target encoded by the nucleic acid molecule encoding a glycoprotein target,
(ii) yield N-acetylgalactosamine (GalNAc) linked to undecaprenyl pyrophosphate (Und-PP), and
(iii) transfer the N-acetylgalactosamine (GalNAc) linked to undecaprenyl pyrophosphate (Und-PP) en bloc to a serine or threonine amino acid of the glycoprotein target.

59. An in vitro method for producing an O-glycosylated protein comprising:
providing reagents suitable for synthesizing a glycoprotein target:
providing glycosylation reagents comprising one more 4-epimerase enzymes, one or more heterologous N,N′-diacetylbacilliosaminyl-1-phosphate transferase enzymes, one or more heterologous O-oligosaccharyltransferases, and one or more ß1,3-galactosyltransferase enzymes capable of transferring galactose (Gal) to undecaprenyl pyrophosphate (Und-PP)-linked N-Acetylgalactosamine (GalNAc),
providing a nucleic acid molecule encoding a glycoprotein target; and
incubating said reagents suitable for synthesizing a glycoprotein target, glycosylation reagents, and nucleic acid molecule encoding a glycoprotein target under conditions effective to:
(i) synthesize the glycoprotein target encoded by the nucleic acid molecule encoding a glycoprotein target,
(ii) yield lipid-linked Gal-ß1,3-GalNAc, and
(iii) transfer the lipid-linked Gal-ß1,3-GalNAc and any additional sugars en bloc to a serine or threonine amino acid of the glycoprotein target.

60. The method of claim 58, wherein the glycosylation reagents further comprise:
(i) the enzymes of an enzymatic pathway capable of producing cytidine-5′-monophosphate-5-N-acetylneuraminic acid (CMP-NeuNAc); and
(ii) one or more α2,6-sialyltransferases, and wherein the N-acetylgalactosamine (GalNAc) linked to undecaprenyl pyrophosphate (Und-PP) is extended by one or more sialic acid (NeuNAc) sugars before the transfer of the N-acetylgalactosamine (GalNAc) linked to undecaprenyl pyrophosphate (Und-PP) en bloc to a serine or threonine amino acid of the glycoprotein target.

61. The method of claim 59, wherein the glycosylation reagents further comprise:
(i) the enzymes of an enzymatic pathway capable of producing cytidine-5′-monophosphate-5-N-acetylneuraminic acid (CMP-NeuNAc); and
(ii) one or more α2,3-sialyltransferases and/or one or more α2,6-sialyltransferases; and wherein the lipid-linked Gal-ß1,3-GalNAc is extended by one or more sialic acid (NeuNAc) sugars before the transfer the lipid-linked Gal-ß1,3-GalNAc en bloc to a serine or threonine amino acid of the glycoprotein target.

62. The recombinant prokaryotic host cell of any one of claims 1-26, wherein the recombinant prokaryotic host cell further expression a glycoprotein target comprising one or more serine and/or threonine residues.

63. The prokaryotic host cell of any one of claims 1-25 or 62, or the method of any one of claims 26-41, wherein expression of one or more of: the one or more of the one more 4-epimerases, one or more glycosyl-1-phosphate transferases, one or more O-oligosaccharyltransferases, one or more ß1,3-galactosyltransferase enzymes capable of transferring galactose (Gal) to undecaprenyl pyrophosphate (Und-PP)-linked N-Acetylgalactosamine (GalNAc), glycoprotein target comprising one or more serine and/or threonine residues, one or more α2,3-sialyltransferases, and one or more α2,6-sialyltransferases is constitutive.

64. The prokaryotic host cell of any one of claims 1-25 or 62, or the method of any one of claims 26-41, wherein expression of one more of: the one or more of the one more 4-epimerases, one or more glycosyl-1-phosphate transferases, one or more O-oligosaccharyltransferases, one or more ß1,3-galactosyltransferase enzymes capable of transferring galactose (Gal) to undecaprenyl pyrophosphate (Und-PP)-linked N-Acetylgalactosamine (GalNAc), glycoprotein target comprising one or more serine and/or threonine residues, one or more α2,3-sialyltransferases, and one or more α2,6-sialyltransferases is inducible.

65. A prokaryotic host cell expressing an α2,6-sialyltransferases and an α2,3-sialyltransferase, wherein the α2,6-sialyltransferases is the α2,6-sialyltransferases from Photobacterium sp. JT-ISH-224 (PspST6) and where the α2,3-sialyltransferase is the α2,3-sialyltransferase from E. coli O104 (EcWbwA).

66. A prokaryotic host cell expressing one or more 4-epimerases, one or more glycosyl-1-phosphate transferases, one or more O-Antigen ligases, and, optionally, one or more ß1,3-galactosyltransferase enzymes capable of transferring galactose (Gal) to undecaprenyl pyrophosphate (Und-PP)-linked N-Acetylgalactosamine (GalNAc).

67. The prokaryotic host cell of claim 66, wherein the prokaryotic host cell does not encode an O-oligosaccharyltransferase.

68. A method for producing a lipid linked Gal-β1,3-GalNAcα (T antigen or core 1) comprising:
providing a recombinant host cell expressing one or more 4-epimerases, one or more glycosyl-1-phosphate transferases, one or more ß1,3-galactosyltransferase enzymes capable of transferring galactose (Gal) to undecaprenyl pyrophosphate (Und-PP)-linked N-Acetylgalactosamine (GalNAc), and one or more O-Antigen ligases (e.g., EcWaaL) and
culturing the host cell under conditions effective to: (i) produce Gal-ß1,3-GalNAc linked to undecaprenyl pyrophosphate (Und-PP) and (ii) transfer Gal-ß1,3-GalNAc linked to undecaprenyl pyrophosphate (Und-PP) en bloc to a lipid target.

69. The method of claim 68, wherein the prokaryotic host cell does not encode an O-oligosaccharyltransferase.

70. The method of claim 68, wherein the recombinant prokaryotic host cell further expresses:
(i) the enzymes of an enzymatic pathway capable of producing cytidine-5′-monophosphate-5-N-acetylneuraminic acid (CMP-NeuNAc), and
(ii) one or more α2,6-sialyltransferases; and wherein the N-acetylgalactosamine (GalNAc) linked to undecaprenyl pyrophosphate (Und-PP) is extended by one or more sialic acid (NeuNAc) sugars before the transfer the N-acetylgalactosamine (GalNAc) linked to undecaprenyl pyrophosphate (Und-PP) en bloc to the lipid target.


SEQUENCE LISTING (57 sequences)

SEQ ID NO: 1 - protein, 329 aa, Campylobacter jejuni
MKILISGGAGYIGSHTLRQFLKTDHEICVLDNLSKGSKIAIEDLQKIRTFKFFEQDLSDFQGVKALFEREKFDAIVHFAASIEVFESMQNPLKYYMNNTVNTTNLIETCLQTGVNKFIFSSTAATYGEPQTPVVSETSPLAPINPYGRSKLMSEEVLRDASMANPEFKHCILRYFNVAGACMDYTLGQRYPKATLLIKVAAECAAGKRNKLFIFGDDYDTKDGTCIRDFIHVDDISSAHLSALDYLKENESNVFNVGYGHGFSVKEVIEAMKKVSGVDFKVELAPRRAGDPSVLISDASKIRNLTSWQPKYDDLGLICKSAFDWEKQCL

SEQ ID NO: 2 - protein, 211 aa, Acinetobacter baumannii
MVNENMKRLVDIVISLIALTVLSPIFLIVAYKVRKNLGSPIFFYQERPGKDGKLFKMIKFRSMKDAFDAQGNPLPDEARITPFGQKLRSTSLDEMPQLINVLKGDMSVVGPRPMLKDFVALYSPEQARRLEVRPGMTGLAQVSGRNELDYEERFKCDVWYVDNHNIWVDFKIMFKTVKVMLKREGINAPGHVGPSLFKGNDTQENIDSSVK

SEQ ID NO: 3 - protein, 268 aa, Escherichia coli
MKFSVLLSLYYKESALFLHDCLESIASNTCAPDQIVIVFDGYISDDLLHVVNEFSLRLPIDIVRLSNNVGLGKALNHGLQYCRNELIFRMDTDDICLPERFALQLSYMKSHPEVVLLGSAIEEFDNTMKIRQGKRFSVIYHEDIKKFAKKRNPFNHMTVVFRKSVIEKLGGYQHHYLMEDYNLWLRILAADYRTHNLSEVLVNVRAGRNMLCRRKGYSYIKSEILLAKLKYELQLDNLTGVIYTGVIRIIPRILPVSLLKLVYNILRK

SEQ ID NO: 4 - protein, 625 aa, Artificial Sequence
  Neisseria meningitides Pg1L
PAETTVSGAHPAAKLPIYILPCFLWIGIVPFTFALKLKPSPDFYHDAAAAAGLIVLLFLTAGKKLFDVKIPAISFLLFAMAAFWYLQARLMNLIYPGMNDIVSWIFILLAVSAWACRSLVAHFGQERIVTLFAWSLLIGSLLQSCIVVIQFAGWEDTPLFQNIIVYSGQGVIGHIGQRNNLGHYLMWGILAAAYLNGQRKIPAALGVICLIMQTAVLGLVNSRTILTYIAAIALILPFWYFRSDKSNRRTMLGIAAAVFLTALFQFSMNTILETFTGIRYETAVERVANGGFTDLPRQIEWNKALAAFQSAPIFGHGWNSFAQQTFLINAEQHNIYDNLLSNLFTHSHNIVLQLLAEMGISGTLLVAATLLTGIAGLLKRPLTPASLFLICTLAVSMCHSMLEYPLWYVYFLIPFGLMLFLSPAEASDGIAFKKAANLGILTASAAIFAGLLHLDWTYTRLVNAFSPATDDSAKTLNRKINELRYISANSPMLSFYADFSLVNFALPEYPETQTWAEEATLKSLKYRPHSATYRIALYLMRQGKVAEAKQWMRATQSYYPYLMPRYADEIRKLPVWAPLLPELLKDCKAFAAAPGHPEAKPCKGSGYPYDVPDYAHHHHHHHHHH

SEQ ID NO: 5 - protein, 623 aa, Artificial Sequence
  Neisseria meningitides PglO
MSAETTVSGARPAAKLPIYILPCFLWIGIIPFTFALRLKPSPDFYHDAAAAAGLIVLLFLTAGKKLFDVKIPAISFLLFAMAAFWWLQARLMNLIYPGMNDIASWVFILLAVSAWACKSLVAHYGQERIVTLFAWSLLIGSLLQSCIVVIQFAGWENTPLLQNIIVHRGQGVIGHIGQRNNLGHYLMWGILASAYLNGQRKIPAALGAICLIMQTAVLGLVNSRTILTYIAAIALILPFWYFRSDKSNRRTMLGIAAAVFLTALFQFSMNAILETFTGIRYETAVERVANGGFTDLPRQSEWNKALAAFQSAPIFGHGWNSFAQQTFLINAEQHTIHDNFLSTLFTHSHNIILQLLAEMGISGTLLVAATLLTGIAGLLKRSLTPASLFLLCALAVSMCHSMLEYPLWYVYFLIPFGLMLFLSPAEASDGIAFKKAANLGILTASAAIFAGLLHLDWTYTRLVNSFSPAADDSAKTLNRKINELRYISANSPMLSFYADFSLVNFALPEYPETQTWAEEATLKALKYRPYSATYRIALYLMRQGKVAEAKQWMRATQSYYPYLMPRYADEIRKLPVWAPLLPELLKDCKAFAAAPGHPETKPCKYPYDVPDYAHHHHHHHHHH

SEQ ID NO: 6 - DNA, 988 bp, Campylobacter jejuni
atgaaaattcttattagcggtggtgcaggttatataggttctcatactttaagacaatttttaaaaacagatcatgaaatttgtgttttagataatctttctaagggttctaaaatcgcaatagaagatttgcaaaaaataagaacttttaaattttttgaacaagatttaagtgattttcaaggcgtaaaagcattgtttgagagagaaaaatttgacgctattgtgcattttgcagcgagcattgaagtttttgaaagtatgcaaaaccctttaaagtattatatgaataacactgttaatacgacaaatctcatcgaaacttgtttgcaaactggagtgaataaatttatattttcttcaacggcagccacttatggcgaaccacaaactcccgttgtgagcgaaacaagtcctttagcacctattaatccttatgggcgtagtaagcttatgagcgaagaggttttgcgtgatgcaagtatggcaaatcctgaatttaagcattgtattttaagatattttaatgttgcaggtgcttgcatggattatactttaggacaacgctatccaaaagcgactttgcttataaaagttgcagctgaatgtgccgcaggaaaacgtaataaacttttcatatttggcgatgattatgatacaaaagatggcacttgcataagagattttatccatgtggatgatatttcaagtgcgcatttatcggctttggattatttaaaagagaatgaaagcaatgtttttaatgtaggttatggacatggttttagcgtaaaagaagtgattgaagcgatgaaaaaagttagcggagtggattttaaagtagaacttgccccacgccgtgcgggtgatcctagtgtattgatttctgatgcaagtaaaatcagaaatcttacttcttggcagcctaaatatgatgatttagggcttatttgtaaatctgcttttgattgggaaaaacagtgcttaa

SEQ ID NO: 7 - DNA, 636 bp, Acinetobacter baumannii
atggtgaatgaaaatatgaagcgtttagtagacatagtcatttctttaatagctttaactgttctgtcgccaatatttctgatagttgcttataaagtccgtaaaaatttaggttcaccaatattcttttaccaagaaagacctggtaaggacggaaaattatttaaaatgattaagttccgttctatgaaagatgcatttgatgctcaaggaaatccattgccagatgaagctcgtattacaccatttggtcaaaaattgcgttcaactagtctggatgaaatgccgcagctcattaatgtactaaaaggtgacatgagcgtagtgggtccgcgtccaatgttaaaagactttgttgctttatattcacccgaacaagctcgtcgtttagaagttcgcccaggaatgacgggtttagctcaggtaagtggtcgtaatgaacttgattatgaagaacgatttaagtgtgatgtatggtatgtagataaccacaacatttgggttgatttcaaaatcatgtttaaaacagtcaaagtgatgttaaaacgtgaaggaatcaatgctccagggcatgttgggccatctttatttaaaggtaatgatacccaagaaaatattgattcttctgttaagtaa

SEQ ID NO: 8 - DNA, 807 bp, Escherichia coli
atgaaatttagcgttctgttatccctgtattacaaagagtctgcactttttctgcatgattgtttagaaagtatagcaagcaatacatgcgcacctgatcaaattgtaattgtctttgatggttatattagtgatgatttgctacatgttgttaatgaattttcattacgtttacccattgatattgtgcgattaagtaataatgttggtctaggtaaggctcttaatcatggtctacagtattgtcgcaatgaactgatttttaggatggatactgatgatatttgtttgcctgaacggttcgctcttcaacttagctatatgaaatcacaccctgaagttgttcttttgggaagtgccattgaagaatttgataatacaatgaaaattaggcaaggaaaaagattttcagttatataccatgaagatataaaaaaatttgcaaaaaaaagaaatcctttcaaccatatgactgttgttttcagaaagtccgtaattgaaaagcttggtggatatcaacatcattatctaatggaagattataacttatggcttaggatacttgcagccgattatcgcactcataatttgagtgaggttcttgttaatgttcgggcaggcagaaatatgctgtgtcgacggaaaggttattcctatattaagagtgagatacttttggcgaaattaaaatatgaattacagttggataatctgacaggtgtcatatatacaggtgtaattcgtattatccctagaatattgcctgtttcattactgaaattagtatacaatatattgcggaaataa

SEQ ID NO: 9 - DNA, 1881 bp, Artificial Sequence
  Neisseria meningitides Pg1L
atgcctgcagaaaccaccgttagcggtgcacatccggcagcaaaactgccgatttatatcctgccgtgttttctgtggattggtattgttccgtttacctttgcactgaaactgaaaccgagtccggatttttatcatgatgcagcagcagccgcaggtctgattgttctgctgtttctgaccgcaggtaaaaaactgttcgatgttaaaattccggcaatcagcttcctgctgtttgcaatggcagcattttggtatctgcaagcacgtctgatgaatctgatttatccgggtatgaatgatatcgtgagctggatctttattctgctggcagttagcgcatgggcatgtcgtagcctggttgcacattttggtcaagaacgtattgttaccctgtttgcatggtcactgctgattggtagcctgctgcagagctgtattgttgttattcagtttgcaggttgggaagatacaccgctgtttcagaacattattgtttatagcggtcagggtgtgattggtcatattggtcagcgtaataatctgggccattatctgatgtggggtattctggcagcagcatatctgaatggtcagcgcaaaattcctgcagcactgggtgttatttgtctgattatgcagaccgcagttctgggtctggttaatagccgtaccattctgacctatattgcagcaattgcactgattctgccgttttggtattttcgtagcgataaaagcaatcgtcgtaccatgctgggtattgccgcagcagtttttctgacagcactgtttcagttcagcatgaatacaatcctggaaacctttaccggtattcgttatgaaaccgcagttgaacgtgttgcaaatggtggttttaccgatctgcctcgtcagattgaatggaataaagcactggcagcctttcagagcgcaccgatttttggtcatggttggaatagctttgcacagcagacctttctgattaatgccgaacagcataacatctatgataacctgctgagcaacctgtttacccatagccataatattgtgctgcagctgctggccgaaatgggtattagcggcaccctgctggttgcagcaaccctgctgaccggtattgcgggtctgctgaaacgtccgctgacaccggcaagcctgtttctgatttgtaccctggccgttagcatgtgtcatagcatgctggaatatccgctgtggtatgtgtattttctgattccgtttggtctgatgctgttcctgagccctgccgaagcaagtgatggtattgcattcaaaaaagcagccaatctgggtatcctgaccgcaagcgcagcaatttttgcaggactgctgcacctggattggacctatacccgtctggtgaatgcatttagtccggcaaccgatgatagcgcaaaaaccctgaatcgtaaaatcaatgaactgcgctatattagcgccaatagcccgatgctgagcttttatgcagattttagcctggtgaattttgccctgccggaatatcctgaaacccagacctgggcagaagaagccaccctgaaaagcctgaaatatcgtccgcatagcgcaacctatcgtattgcactgtatctgatgcgtcagggtaaagttgcggaagcaaaacagtggatgcgtgcaacccagagctattatccgtacctgatgcctcgttatgccgatgaaattcgtaaactgccggtttgggcaccgctgctgcctgaactgctgaaagattgtaaagcatttgcagccgcaccgggtcatccggaagccaaaccgtgtaaaggtagcggttatccgtatgatgttccggattatgcccatcaccatcaccatcaccatcaccatcactaa

SEQ ID NO: 10 - DNA, 1872 bp, Artificial Sequence
  Neisseria meningitides PglO
atgagcgcagaaaccaccgttagcggtgcacgtccggcagcaaaactgccgatttatatcctgccgtgttttctgtggattggcattattccgtttacctttgcactgcgtctgaaaccgagtccggatttttatcatgatgcagcagcagccgcaggtctgattgttctgctgtttctgaccgcaggtaaaaaactgttcgatgttaaaattccggcaatcagcttcctgctgtttgcaatggcagcattttggtggctgcaggcacgtctgatgaatctgatttatccgggtatgaatgatattgccagctgggtttttattctgctggcagttagcgcatgggcatgtaaaagcctggttgcacattatggtcaagaacgtattgttaccctgtttgcatggtcactgctgattggtagcctgctgcagagctgtattgttgttattcagtttgcaggctgggaaaatacaccgctgctgcaaaacattattgttcatcgtggtcagggtgtgattggtcatattggtcagcgtaataatctgggccattatctgatgtggggtattctggcaagcgcatatctgaatggtcagcgcaaaattcctgcagcactgggtgcaatttgtctgattatgcagaccgcagttctgggtctggttaatagccgtaccattctgacctatattgcagcaattgcactgattctgccgttttggtattttcgtagcgataaaagcaatcgtcgtaccatgctgggtattgccgcagcagtttttctgacagcactgtttcagtttagcatgaatgcaatcctggaaacctttaccggtattcgttatgaaaccgcagttgaacgtgttgcaaatggtggttttaccgatctgcctcgtcagagcgaatggaataaagcactggcagcctttcagagcgcaccgatttttggtcatggttggaatagctttgcacagcagacctttctgattaatgccgaacagcataccatccacgataattttctgagcaccctgtttacccatagccataacattattctgcagctgctggccgaaatgggtattagcggcaccctgctggttgcagcaaccctgctgaccggtattgcgggtctgctgaaacgtagcctgacaccggcaagcctgtttctgctgtgtgcactggccgttagcatgtgtcatagcatgctggaatatccgctgtggtatgtttattttctgattccgtttggcctgatgctgttcctgagccctgccgaagcaagtgatggtattgccttcaaaaaagcagcaaatctgggtatcctgaccgcaagcgcagcaatttttgcaggactgctgcacctggattggacctatacccgtctggtgaatagtttttcaccggcagcagatgatagcgcaaaaaccctgaatcgtaaaatcaatgaactgcgctatattagcgccaatagcccgatgctgagcttttatgcagattttagcctggttaattttgccctgccggaatatcctgaaacccagacctgggcagaagaagccaccctgaaagcactgaaatatcgtccgtatagcgcaacctatcgtattgcactgtatctgatgcgtcagggtaaagttgcggaagcaaaacagtggatgcgtgcaacccagagctattatccgtacctgatgcctcgttatgccgatgaaattcgtaaactgccggtttgggcacctctgctgcctgaactgctgaaagattgtaaagcatttgcagccgcaccgggtcatccggaaaccaaaccgtgtaaatatccgtatgatgttccggattatgcgcatcatcaccaccatcaccatcatcatcactaa

SEQ ID NO: 11 - protein, 418 aa, Escherichia coli
MRTKIIAIIPARSGSKGLRNKNALMLIDKPLLAYTIEAALQSEMFEKVIVTTDSEQYGAIAESYGADFLLRPEELATDKASSFEFIKHALSIYTDYENFALLQPTSPFRDSTHIIEAVKLYQTLEKYQCVVSVTRSNKPSQIIRPLDDYSTLSFFDLDYSKYNRNSIVEYHPNGAIFIANKQHYLHTKHFFGRYSLAYIMDKESSLDIDDRMDFELAITIQQKKNRQKILYQNIHNRINEKRNEFDSVSDITLIGHSLFDYWDVKKINDIEVNNLGIAGINSKEYYEYIIEKERIVNFGEFVFIFFGTNDIVVSDWKKEDTLWYLKKTCQYIKKKNAASKIYLLSVPPVFGRIDRDNRIINDLNSYLRENVDFAKFISLDHVLKDSYGNLNKMYTYDGLHFNSNGYTVLENEIAEIVK

SEQ ID NO: 12 - protein, 323 aa, Escherichia coli
MILKAKEAGVNAVKFQTFKADKLISAIAPKAEYQIKNTGELESQLEMTKKLEMKYDDYLHLMEYAVSLNLDVFSTPFDEDSIDFLASLKQKIWKIPSGELLNLPYLEKIAKLPIPDKKIIISTGMATIDEIKQSVSIFINNKVPVDNITILHCNTEYPTPFEDVNLNAINDLKKHFPKNNIGFSDHSSGFYAAIAAVPYGITFIEKHFTLDKSMSGPDHLASIEPDELKHLCIGVRCVEKSLGSNSKVVTASERKNKIVARKSIIAKTEIKKGEVFSEKNITTKRPGNGISPMEWYNLLGKIAEQDFIPDELIIHSEFKNQGE

SEQ ID NO: 13 - protein, 391 aa, Escherichia coli
MKKILYVTGSRAEYGIVRRLLTMLRETPEIQLDLAVTGMHCDNAYGNTIHIIEQDNFNIIKVVDININTTSHTHILHSMSVCLNSFGDFFSNNTYDAVMVLGDRYEIFSVAIAASMHNIPLIHIHGGEKTLANYDEFIRHSITKMSKLHLTSTEEYKKRVIQLGEKPGSVFNIGSLGAENALSLHLPNKQELELKYGSLLKRYFVVVFHPETLSTQSVNDQIDELLSAISFFKNTHDFIFIGSNADTGSDIIQRKVKYFCKEYKFRYLISIRSEDYLAMIKCSCGLIGNSSSGLIEVPSLKVATINIGDRQKGRVRGASVIDVPVEKNAIVRGINISQDEKFISVVQSSSNPYFKENALINAVRIIKDFIKSKNKDYKDFYDIPECTTSYD

SEQ ID NO: 14 - protein, 207 aa, Escherichia coli
MSKKLIIFGAGGFSKSIIDSLNHKHYELIGFIDKYKSGYHQSYPILGNDIADIENKDNYYYFIGIGKPSTRKHYLNIIRKHNLRLINIIDKTAILSPNIILGDGIFIGKMCILNRDTRIHDAVVINTRSLIEHGNEIGCCSNISTNVVLNGDVSVGEETFVGSCTVVNGQLKLGSKSIIGSGSVVIRNIPSNVVVAGTPTRLIRGNE

SEQ ID NO: 15 - DNA, 1257 bp, Escherichia coli
atgagaacaaaaattattgcgataattccagcccgtagtggatctaaagggttgagaaataaaaatgctttgatgctgatagataaacctcttcttgcttatacaattgaagctgccttgcagtcagaaatgtttgagaaagtaattgtgacaactgactccgaacagtatggagcaatagcagagtcatatggtgctgattttttgctgagaccggaagaactagcaactgataaagcatcatcatttgaatttataaaacatgcgttaagtatatatactgattatgagaactttgctttattacaaccaacttcaccctttagagattcgacccatattattgaggctgtaaagttatatcaaactttagaaaaataccaatgtgttgtttctgttactagaagcaataagccatcacaaataattagaccattagatgattactcgacactgtctttttttgaccttgattatagtaaatataatcgaaactcaatagtagaatatcatccgaatggagctatatttatagctaataagcagcattatcttcatacaaagcatttttttggtcgctattcactagcttatattatggataaggaaagctctttagatatagatgatagaatggatttcgaacttgcaattaccattcagcaaaaaaaaaatagacaaaaaatactttatcaaaacatacataatagaatcaatgagaaacgaaatgaatttgatagtgtaagtgatataactttaattggacactcgctgtttgattattgggacgtaaaaaaaataaatgatatagaagttaataacttaggtatcgctggtataaactcgaaggagtactatgaatatattattgagaaagagcggattgttaatttcggagagtttgttttcatcttttttggaactaatgatatagttgttagtgattggaaaaaagaagacacattgtggtatttgaagaaaacatgccagtatataaagaagaaaaatgctgcatcaaaaatttatttattgtcggttcctcctgtttttgggcgtattgatcgagataatagaataattaatgatttaaattcttatcttcgagagaatgtagattttgcgaagtttattagcttggatcacgttttaaaagactcttatggcaatctaaataaaatgtatacttatgatggcttacattttaatagtaatgggtatacagtattagaaaacgaaatagcggagattgttaaatga

SEQ ID NO: 16 - DNA, 972 bp, Escherichia coli
atgatattaaaagccaaagaggccggtgttaatgcagtaaaattccaaacatttaaagctgataaattaatttcagctattgcacctaaggcagagtatcaaataaaaaacacaggagaattagaatctcagttagaaatgacaaaaaagcttgaaatgaagtatgacgattatctccatctaatggaatatgcagtcagtttaaatttagatgttttttctaccccttttgacgaagactctattgattttttagcatctttgaaacaaaaaatatggaaaatcccttcaggtgagttattgaatttaccgtatcttgaaaaaatagccaagcttccgatccctgataagaaaataatcatatcaacaggaatggctactattgatgagataaaacagtctgtttctatttttataaataataaagttccggttgataatattacaatattacattgcaatactgaatatccaacgccctttgaggatgtaaaccttaatgctattaatgatttgaaaaaacacttccctaagaataacataggcttctctgatcattctagcgggttttatgcagctattgcggcggtgccttatggaataacttttattgaaaaacatttcactttagataaatctatgtctggcccagatcatttggcctcaatagaacctgatgaactgaaacatctatgtattggggtcaggtgtgttgaaaaatctttaggttcaaatagtaaagtggttacagcttcagaaaggaagaataaaatcgtagcaagaaagtctattatagctaaaacagagataaaaaaaggtgaggttttttcagaaaaaaatataacaacaaaaagacctggtaatggtatcagtccgatggagtggtataatttattgggtaaaattgcagagcaagactttattccagatgaattaataattcatagcgaattcaaaaatcagggggaataa

SEQ ID NO: 17 - DNA, 1176 bp, Escherichia coli
atgaaaaaaatattatacgtaactggatctagagctgaatatggaatagttcggagacttttgacaatgctaagagaaactccagaaatacagcttgatttggcagttacaggaatgcattgtgataatgcgtatggaaatacaatacatattatagaacaagataattttaatattatcaaggttgtggatataaatatcaatacaacttcacatactcacattctccattcaatgagtgtttgcctcaattcgtttggtgattttttttcaaataacacatatgatgcggttatggttttaggcgatagatatgaaatattttcagtcgctatcgcagcatcaatgcataatattccattaattcatattcatggtggtgaaaagacattagctaattatgatgagtttattaggcattcaattactaaaatgagtaaactccatcttacttctacagaagagtataaaaaacgagtaattcaactaggtgaaaagcctggtagtgtgtttaatattggttctcttggtgcagaaaatgctctttcattgcatttaccaaataagcaggagttggaactaaaatatggttcactgttaaaacggtactttgttgtagtattccatcctgaaacactttccacgcagtcggttaatgatcaaatagatgagttattgtcagcgatttctttttttaaaaatactcacgactttatttttattggcagtaacgctgacactggttctgatataattcagagaaaagtaaaatatttttgcaaagagtataagttcagatatttgatttctattcgttcagaagattatttggcaatgattaaatgctcttgtgggctaattgggaactcctcctctggtttaattgaggttccatctttaaaagttgcaacaattaacattggtgataggcagaaaggccgtgttcgtggagccagtgtaatagatgtacccgttgaaaaaaatgcaatcgtcagagggataaatatatctcaagatgaaaaatttattagtgttgtacagtcatctagtaatccttattttaaagaaaatgctttaattaatgctgttagaattattaaggattttattaaatcaaaaaataaagattacaaagatttttatgacatcccggaatgtaccaccagttatgactag

SEQ ID NO: 18 - DNA, 624 bp, Escherichia coli
atgagtaaaaaattaataatatttggtgcgggtggtttttcaaaatctataattgacagcttaaatcataaacattacgagttaataggatttatcgataaatataaaagtggttatcatcaatcatatccaatattaggtaatgatattgcagacatcgagaataaggataattattattattttattgggataggcaaaccatcaactaggaagcactatttaaacatcataagaaaacataatctacgcttaattaacattatagataaaactgctattctatcaccaaatattatactgggtgatggaatttttattggtaaaatgtgtatacttaaccgtgatactagaatacatgatgccgttgtaataaatactaggagtttaattgaacatggtaatgaaataggctgctgtagcaatatctctactaatgttgtacttaatggtgatgtttctgttggagaagaaacttttgttggtagctgtactgttgtaaatggccagttgaagctaggctcaaagagtattattggttctgggtcggttgtaattagaaatataccaagtaatgttgtagttgctgggactccaacaagattaattagggggaatgaatga

SEQ ID NO: 19 - protein, 498 aa, Photobacterium sp. JT-ISH-224
MSEENTQSIIKNDINKTIIDEEYVNLEPINQSNISFTKHSWVQTCGTQQLLTEQNKESISLSVVAPRLDDDEKYCFDFNGVSNKGEKYITKVTLNVVAPSLEVYVDHASLPTLQQLMDIIKSEEENPTAQRYIAWGRIVPTDEQMKELNITSFALINNHTPADLVQEIVKQAQTKHRLNVKLSSNTAHSFDNLVPILKELNSFNNVTVTNIDLYDDGSAEYVNLYNWRDTLNKTDNLKIGKDYLEDVINGINEDTSNTGTSSVYNWQKLYPANYHFLRKDYLTLEPSLHELRDYIGDSLKQMQWDGFKKFNSKQQELFLSIVNFDKQKLQNEYNSSNLPNFVFTGTTVWAGNHEREYYAKQQINVINNAINESSPHYLGNSYDLFFKGHPGGGIINTLIMQNYPSMVDIPSKISFEVLMMTDMLPDAVAGIASSLYFTIPAEKIKFIVFTSTETITDRETALRSPLVQVMIKLGIVKEENVLFWADLPNCETGVCIAV

SEQ ID NO: 20 - protein, 315 aa, Escherichia coli
MKMRNNVFVFDSPYCLLIYCILFNHQLHDTIYIYSDNVSLRDINFPGKERYIIKKGNNKTSKIFYYFCFLFRLIINPKLRKLISNRKDYRFLGQDHLFFSKPFLNEFILLEDGLANYRYPHYSRLYKLIIGGHTFGRSTKVNKILLSGMIDIIDPTINDKVEFFDLIAAWEKLNSVQKKEINHIFNYCIEDELVADVMILTQPLSEDGFISENEKIRLYDEIIKEYDDKKIVIRQHPRELTDYSLYFKDVKVNKVKAPVELVVLNSPMIKTAVTLFSGGIFNIPCREKIFKGTSFSPLLSKHFGNISNKFEVKGK

SEQ ID NO: 21 - DNA, 1497 bp, Photobacterium sp. JT-ISH-224
atgagcgaagaaaatacccagagcatcatcaaaaacgatatcaacaaaaccatcatcgacgaagagtatgttaacctggaaccgattaatcagagcaacatcagctttaccaaacatagctgggttcagacctgtggcacccagcaactgctgacagaacagaataaagaaagcattagcctgagcgttgttgcaccgcgtctggatgatgatgagaaatattgctttgatttcaacggcgtgagcaacaaaggcgaaaaatacattaccaaagtgaccctgaatgttgtggcaccgagcctggaagtttatgttgatcatgcaagcctgccgaccctgcagcagctgatggatattatcaaaagcgaagaagaaaatccgaccgcacagcgttatattgcatggggtcgtattgttccgaccgatgagcagatgaaagaactgaatattaccagctttgccctgatcaataatcatacaccggcagatctggttcaagaaattgttaaacaggcacagaccaaacatcgtctgaatgttaaactgagcagcaataccgcacatagctttgataatctggttccgattctgaaagagctgaacagctttaataacgtgaccgtgaccaatatcgatctgtacgatgatggcagcgcagaatatgtgaatctgtataattggcgtgacaccctgaataaaaccgacaatctgaaaatcggcaaagattacctggaagatgtgattaacggcatcaatgaagataccagtaataccggcaccagcagcgtttataactggcagaaactgtatccggcaaactatcattttctgcgcaaagactatctgacactggaaccgtccctgcatgaactgcgtgattatattggtgatagcctgaaacaaatgcagtgggatggtttcaaaaaattcaacagcaaacagcaagaactgttcctgagcattgtgaacttcgataaacagaaactgcagaacgaatacaatagcagcaatctgccgaattttgtgtttaccggtacaaccgtttgggcaggtaatcatgaacgcgaatattatgcaaaacagcagatcaacgtgatcaacaacgcaattaatgaaagcagtccgcattatctgggtaatagctatgacctgtttttcaaaggtcatccgggtggtggtattatcaataccctgattatgcagaattatccgagcatggttgatatcccgagcaaaattagctttgaagttctgatgatgaccgatatgctgccggatgccgttgcaggtattgcaagcagcctgtattttaccattccggcagaaaaaatcaaattcatcgttttcaccagcaccgaaaccattaccgatcgtgaaaccgcactgcgtagtccgctggttcaggttatgattaaactgggtattgtgaaagaggaaaacgtcctgttttgggcagacctgccgaactgtgaaaccggtgtttgtattgcagtttaa

SEQ ID NO: 22 - DNA, 951 bp, Escherichia coli
atgaagatgcgtaacaacgtattcgtcttcgactcaccgtactgccttttaatttattgcatccttttcaaccatcagcttcacgatacaatctacatttactcggacaacgtctcattgcgtgatattaactttcctgggaaagagcgctatatcatcaaaaaagggaacaacaaaacaagtaaaatcttctattacttctgcttcttgttccgtcttatcattaaccctaagctgcgtaaattaatctctaatcgtaaagactaccgcttccttggccaggaccacttgttcttttcaaagcctttcttaaatgagttcatcctgcttgaggatggtttagccaactatcgttatcctcactactctcgcctttacaagttaatcattggggggcacacgtttggccgtagtaccaaggtaaacaagatcttgttaagtggaatgatcgacatcattgaccccactatcaatgacaaggtggagtttttcgacttgatcgctgcgtgggagaagctgaacagtgtgcagaagaaggaaattaatcatatcttcaactattgtatcgaggatgaacttgttgctgatgtgatgattctgactcaaccattgagcgaagacggctttatctcggagaatgaaaaaattcgcttgtatgacgaaatcattaaagaatacgacgataaaaaaattgttatccgccaacacccgcgtgagttgaccgattactccttgtattttaaggacgtcaaggttaataaagtaaaggcccctgttgaattagtcgtacttaatagcccgatgattaagaccgctgtaactttattcagcggtggaatcttcaacattccatgtcgtgagaaaatctttaagggaacctcgttttctcctcttctgagtaaacacttcggtaatatcagtaacaagttcgaggtaaagggaaagaaataa

SEQ ID NO: 23 - protein, 22 aa, Artificial Sequence
  Glycosylated MBPMOOR peptide
  CARBOHYD (13)..(13): amino acid at position 13 is modified with a HexNAc monosaccharide
NVGGDLDWPAAASAPQPGKPPR

SEQ ID NO: 24 - protein, 8 aa, Artificial Sequence
  MBPMOOR core sequence
WPAAASAP

SEQ ID NO: 25 - protein, 22 aa, Artificial Sequence
  Glycosylated MBPMOOR peptide
  CARBOHYD (13)..(13): amino acid at position 13 is modified with a HexHexNAc disaccharide
NVGGDLDWPAAASAPQPGKPPR

SEQ ID NO: 26 - protein, 22 aa, Artificial Sequence
  Glycosylated MBPMOOR peptide
  CARBOHYD (13)..(13): amino acid at position 13 is modified with a NeuNAcHexHexNAc oligosaccharide
NVGGDLDWPAAASAPQPGKPPR

SEQ ID NO: 27 - protein, 10 aa, Artificial Sequence
  hydrophilic flanking sequence
DPRNVGGDLD

SEQ ID NO: 28 - protein, 7 aa, Artificial Sequence
  hydrophilic flanking sequence
QPGKPPR

SEQ ID NO: 29 - protein, 7 aa, Artificial Sequence
  MUC1 minimal epitope motif
PPAHGVT

SEQ ID NO: 30 - protein, 5 aa, Artificial Sequence
  MUC1 minimal epitope motif
PDTRP

SEQ ID NO: 31 - protein, 6 aa, Artificial Sequence
  MUC1 minimal epitope motif
RPAPGS

SEQ ID NO: 32 - protein, 9 aa, Artificial Sequence
  MHC class I binding epitope
STAPPAHGV

SEQ ID NO: 33 - protein, 9 aa, Artificial Sequence
  MHC class I binding epitope
SAPDTRPAP

SEQ ID NO: 34 - protein, 9 aa, Artificial Sequence
  MHC class I binding epitope
TSAPDTRPA

SEQ ID NO: 35 - protein, 9 aa, Artificial Sequence
  MHC class I binding epitope
APDTRPAPG

SEQ ID NO: 36 - protein, 20 aa, Artificial Sequence
  MUC1 repeat sequence
PDTRPAPGSTAPPAHGVTSA

SEQ ID NO: 37 - protein, 22 aa, Artificial Sequence
  MBPMOOR peptide
  CARBOHYD (13)..(13): amino acid at position 13 is modified
NVGGDLDWPAAASAPQPGKPPR

SEQ ID NO: 38 - protein, 25 aa, Artificial Sequence
  EPO
DPRNVGGDLDPPDAASAAQPGKPPR

SEQ ID NO: 39 - protein, 25 aa, Artificial Sequence
  GPC
DPRNVGGDLDDPGMASASQPGKPPR

SEQ ID NO: 40 - protein, 26 aa, Artificial Sequence
  SAP
DPRNVGGDLDSAPSAPSAPQPGKPPR

SEQ ID NO: 41 - protein, 25 aa, Artificial Sequence
  MUC1_8
DPRNVGGDLDAHGVTSAPQPGKPPR

SEQ ID NO: 42 - protein, 29 aa, Artificial Sequence
  MUC1_12
DPRNVGGDLDPPAHGVTSAPDTQPGKPPR

SEQ ID NO: 43 - protein, 33 aa, Artificial Sequence
  MUC1_16
DPRNVGGDLDTAPPAHGVTSAPDTRPQPGKPPR

SEQ ID NO: 44 - protein, 37 aa, Artificial Sequence
  MUC1_20
DPRNVGGDLDGSTAPPAHGVTSAPDTRPAPQPGKPPR

SEQ ID NO: 45 - protein, 41 aa, Artificial Sequence
  MUC1_24
DPRNVGGDLDAPGSTAPPAHGVTSAPDTRPAPGSQPGKPPR

SEQ ID NO: 46 - protein, 41 aa, Artificial Sequence
  MUC1_41
GVTSAPDTRPAPGSTAPPAHGVTSAPDTRPAPGSTAPPAHG

SEQ ID NO: 47 - protein, 22 aa, Artificial Sequence
  MUC1_8
  CARBOHYD (13)..(13): amino acid at position 13 is modified with a HexNAcHex oligosaccharide
NVGGDLDAHGVTSAPQPGKPPR

SEQ ID NO: 48 - protein, 1255 aa, Artificial Sequence
  Mucin 1
MTPGTQSPFFLLLLLTVLTVVTGSGHASSTPGGEKETSATQRSSVPSSTEKNAVSMTSSVLSSHSPGSGSSTTQGQDVTLAPATEPASGSAATWGQDVTSVPVTRPALGSTTPPAHDVTSAPDNKPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDTRPAPGSTAPPAHGVTSAPDNRPALGSTAPPVHNVTSASGSASGSASTLVHNGTSARATTTPASKSTPFSIPSHHSDTPTTLASHSTKTDASSTHHSSVPPLTSSNHSTSPQLSTGVSFFFLSFHISNLQFNSSLEDPSTDYYQELQRDISEMFLQIYKQGGFLGLSNIKFRPGSVVVQLTLAFREGTINVHDVETQFNQYKTEAASRYNLTISDVSVSDVPFPFSAQSGAGVPGWGIALLVLVCVLVALAIVYLIALAVCQCRRKNYGQLDIFPARDTYHPMSEYPTYHTHGRYVPPSSTDRSPYEKVSAGNGGSSLSYTNPAVAAASANL

SEQ ID NO: 49 - protein, 400 aa, Artificial Sequence
  Leukosialin
MATLLLLLGVLVVSPDALGSTTAVQTPTSGEPLVSTSEPLSSKMYTTSITSDPKADSTGDQTSALPPSTSINEGSPLWTSIGASTGSPLPEPTTYQEVSIKMSSVPQETPHATSHPAVPITANSLGSHTVTGGTITTNSPETSSRTSGAPVTTAASSLETSRGTSGPPLTMATVSLETSKGTSGPPVTMATDSLETSTGTTGPPVTMTTGSLEPSSGASGPQVSSVKLSTMMSPTTSTNASTVPFRNPDENSRGMLPVAVLVALLAVIVLVALLLLWRRRQKRRTGALVLSRGGKRNGVVDAWAGPAQVPEEGAVTVTVGGSGGDKGSGFPDGEGSSRRPTLTTFFGRRKSRQGSLAMEELKSGSGPSLKGEEEPLVASEDGAVDAPAPDEPEGGDGAAP

SEQ ID NO: 50 - protein, 150 aa, Artificial Sequence
  Glycophorin A
MYGKIIFVLLLSAIVSISASSTTGVAMHTSTSSSVTKSYISSQTNDTHKRDTYAATPRAHEVSEISVRTVYPPEEETGERVQLAHHFSEPEITLIIFGVMAGVIGTILLISYGIRRLIKKSPSDVKPLPSPDTDVPLSSVEIENPETSDQ

SEQ ID NO: 51 - protein, 128 aa, Artificial Sequence
  Glycophorin C
MWSTRSPNSTAWPLSLEPDPGMASASTTMHTTTIAEPDPGMSGWPDGRMETSTPTIMDIVVIAGVIAAVAIVLVSLLFVMLRYMYRHKGTYHTNEAKGTEFAESADAALQGDPALQDAGDSSRKEYFI

SEQ ID NO: 52 - protein, 2813 aa, Artificial Sequence
  Von willebrand factor
MIPARFAGVLLALALILPGTLCAEGTRGRSSTARCSLFGSDFVNTFDGSMYSFAGYCSYLLAGGCQKRSFSIIGDFQNGKRVSLSVYLGEFFDIHLFVNGTVTQGDQRVSMPYASKGLYLETEAGYYKLSGEAYGFVARIDGSGNFQVLLSDRYFNKTCGLCGNFNIFAEDDFMTQEGTLTSDPYDFANSWALSSGEQWCERASPPSSSCNISSGEMQKGLWEQCQLLKSTSVFARCHPLVDPEPFVALCEKTLCECAGGLECACPALLEYARTCAQEGMVLYGWTDHSACSPVCPAGMEYRQCVSPCARTCQSLHINEMCQERCVDGCSCPEGQLLDEGLCVESTECPCVHSGKRYPPGTSLSRDCNTCICRNSQWICSNEECPGECLVTGQSHFKSFDNRYFTFSGICQYLLARDCQDHSFSIVIETVQCADDRDAVCTRSVTVRLPGLHNSLVKLKHGAGVAMDGQDIQLPLLKGDLRIQHTVTASVRLSYGEDLQMDWDGRGRLLVKLSPVYAGKTCGLCGNYNGNQGDDFLTPSGLAEPRVEDFGNAWKLHGDCQDLQKQHSDPCALNPRMTRFSEEACAVLTSPTFEACHRAVSPLPYLRNCRYDVCSCSDGRECLCGALASYAAACAGRGVRVAWREPGRCELNCPKGQVYLQCGTPCNLTCRSLSYPDEECNEACLEGCFCPPGLYMDERGDCVPKAQCPCYYDGEIFQPEDIFSDHHTMCYCEDGFMHCTMSGVPGSLLPDAVLSSPLSHRSKRSLSCRPPMVKLVCPADNLRAEGLECTKTCQNYDLECMSMGCVSGCLCPPGMVRHENRCVALERCPCFHQGKEYAPGETVKIGCNTCVCRDRKWNCTDHVCDATCSTIGMAHYLTFDGLKYLFPGECQYVLVQDYCGSNPGTFRILVGNKGCSHPSVKCKKRVTILVEGGEIELFDGEVNVKRPMKDETHFEVVESGRYIILLLGKALSVVWDRHLSISVVLKQTYQEKVCGLCGNFDGIQNNDLTSSNLQVEEDPVDFGNSWKVSSQCADTRKVPLDSSPATCHNNIMKQTMVDSSCRILTSDVFQDCNKLVDPEPYLDVCIYDTCSCESIGDCACFCDTIAAYAHVCAQHGKVVTWRTATLCPQSCEERNLRENGYECEWRYNSCAPACQVTCQHPEPLACPVQCVEGCHAHCPPGKILDELLQTCVDPEDCPVCEVAGRRFASGKKVTLNPSDPEHCQICHCDVVNLTCEACQEPGGLVVPPTDAPVSPTTLYVEDISEPPLHDFYCSRLLDLVFLLDGSSRLSEAEFEVLKAFVVDMMERLRISQKWVRVAVVEYHDGSHAYIGLKDRKRPSELRRIASQVKYAGSQVASTSEVLKYTLFQIFSKIDRPEASRIALLLMASQEPQRMSRNFVRYVQGLKKKKVIVIPVGIGPHANLKQIRLIEKQAPENKAFVLSSVDELEQQRDEIVSYLCDLAPEAPPPTLPPHMAQVTVGPGLLGVSTLGPKRNSMVLDVAFVLEGSDKIGEADFNRSKEFMEEVIQRMDVGQDSIHVTVLQYSYMVTVEYPFSEAQSKGDILQRVREIRYQGGNRTNTGLALRYLSDHSFLVSQGDREQAPNLVYMVTGNPASDEIKRLPGDIQVVPIGVGPNANVQELERIGWPNAPILIQDFETLPREAPDLVLQRCCSGEGLQIPTLSPAPDCSQPLDVILLLDGSSSFPASYFDEMKSFAKAFISKANIGPRLTQVSVLQYGSITTIDVPWNVVPEKAHLLSLVDVMQREGGPSQIGDALGFAVRYLTSEMHGARPGASKAVVILVTDVSVDSVDAAADAARSNRVTVFPIGIGDRYDAAQLRILAGPAGDSNVVKLQRIEDLPTMVTLGNSFLHKLCSGFVRICMDEDGNEKRPGDVWTLPDQCHTVTCQPDGQTLLKSHRVNCDRGLRPSCPNSQSPVKVEETCGCRWTCPCVCTGSSTRHIVTFDGQNFKLTGSCSYVLFQNKEQDLEVILHNGACSPGARQGCMKSIEVKHSALSVELHSDMEVTVNGRLVSVPYVGGNMEVNVYGAIMHEVRFNHLGHIFTFTPQNNEFQLQLSPKTFASKTYGLCGICDENGANDFMLRDGTVTTDWKTLVQEWTVQRPGQTCQPILEEQCLVPDSSHCQVLLLPLFAECHKVLAPATFYAICQQDSCHQEQVCEVIASYAHLCRTNGVCVDWRTPDFCAMSCPPSLVYNHCEHGCPRHCDGNVSSCGDHPSEGCFCPPDKVMLEGSCVPEEACTQCIGEDGVQHQFLEAWVPDHQPCQICTCLSGRKVNCTTQPCPTAKAPTCGLCEVARLRQNADQCCPEYECVCDPVSCDLPPVPHCERGLQPTLTNPGECRPNFTCACRKEECKRVSPPSCPPHRLPTLRKTQCCDEYECACNCVNSTVSCPLGYLASTATNDCGCTTTTCLPDKVCVHRSTIYPVGQFWEEGCDVCTCTDMEDAVMGLRVAQCSQKPCEDSCRSGFTYVLHEGECCGRCLPSACEVVTGSPRGDSQSSWKSVGSQWASPENPCLINECVRVKEEVFIQQRNVSCPQLEVPVCPSGFQLSCKTSACCPSCRCERMEACMLNGTVIGPGKTVMIDVCTTCRCMVQVGVISGFKLECRKTTCNPCPLGYKEENNTGECCGRCLPTACTIQLRGGQIMTLKRDETLQDGCDTHFCKVNERGEYFWEKRVTGCPPFDEHKCLAEGGKIMKIPGTCCDTCEEPECNDITARLQYVKVGSCKSEVEVDIHYCQGKCASKAMYSIDINDVQDQCSCCSPTRTEPMQVALHCTNGSVVYHEVLNAMECKCSPRKCSK

SEQ ID NO: 53 - protein, 644 aa, Artificial Sequence
  Kininogen
MKLITILFLCSRLLLSLTQESQSEEIDCNDKDLFKAVDAALKKYNSQNQSNNQFVLYRITEATKTVGSDTFYSFKYEIKEGDCPVQSGKTWQDCEYKDAAKAATGECTATVGKRSSTKFSVATQTCQITPAEGPVVTAQYDCLGCVHPISTQSPDLEPILRHGIQYFNNNTQHSSLFMLNEVKRAQRQVVAGLNFRITYSIVQTNCSKENFLFLTPDCKSLWNGDTGECTDNAYIDIQLRIASFSQNCDIYPGKDFVQPPTKICVGCPRDIPTNSPELEETLTHTITKLNAENNATFYFKIDNVKKARVQVVAGKKYFIDFVARETTCSKESNEELTESCETKKLGQSLDCNAEVYVVPWEKKIYPTVNCQPLGMISLMKRPPGFSPFRSSRIGEIKEETTVSPPHTSMAPAQDEERDSGKEQGHTRRHDWGHEKQRKHNLGHGHKHERDQGHGHQRGHGLGHGHEQQHGLGHGHKFKLDDDLEHQGGHVLDHGHKHKHGHGHGKHKNKGKKNGKHNGWKTEHLASSSEDSTTPSAQTQEKTEGPTPIPSLAKPGVTVTFSDFQDSDLIATMMPPISPAPIQSDDDWIPDIQTDPNGLSFNPISDFPDTTSPKCPGRPWKSVSEINPTTQMKESYYFDLTDGLS

SEQ ID NO: 54 - protein, 165 aa, Artificial Sequence
  Chorionic gonadotropin beta chain
MEMFQGLLLLLLLSMGGTWASKEPLRPRCRPINATLAVEKEGCPVCITVNTTICAGYCPTMTRVLQGVLPALPQVVCNYRDVRFESIRLPGCPRGVNPVVSYAVALSCQCALCRRSTTDCGGPKDHPLTCDDPRFQDSSSSKAPPPSLPSPSRLPGPSDTPILPQ

SEQ ID NO: 55 - protein, 34 aa, Artificial Sequence
  MUC1_20
  CARBOHYD (19)..(19): amino acid at position 19 modified with a HexNAcHex oligosaccharide
NVGGDLDGSTAPPAHGVTSAPDTRPAPQPGKPPR

SEQ ID NO: 56 - protein, 38 aa, Artificial Sequence
  MUC1_24
  CARBOHYD (21)..(21): amino acid at position 21 is modified with a HexNAcHex oligosaccharide
NVGGDLDAPGSTAPPAHGVTSAPDTRPAPGSQPGKPPR

SEQ ID NO: 57 - protein, 49 aa, Artificial Sequence
  MUC1_41
  CARBOHYD (24)..(24): amino acid at position 24 is modified with a HexNAcHex oligosaccharide
GVTSAPDTRPAPGSTAPPAHGVTSAPDTRPAPGSTAPPAHGVTHHHHHH